SEQUENCE LISTING (10 sequences)

SEQ ID NO: 1 - RNA, 21 nt, synthetic construct
  source 1..21: mol_type = other RNA
ctagacctgttttgcttttgt

SEQ ID NO: 2 - RNA, 23 nt, synthetic construct
  source 1..23: mol_type = other RNA
acaaaagcaaaacaggtctagaa

SEQ ID NO: 3 - RNA, 21 nt, synthetic construct
  source 1..21: mol_type = other RNA
  modified_base 1: mod_base = OTHER; note = 2 prime-O-methylcytidine-3 prime-phosphorothioate
  modified_base 2: mod_base = OTHER; note = 2 prime-O-methyluridine-3 prime-phosphorothioate
  modified_base order(3,5): mod_base = OTHER; note = 2 prime-O-methyladenosine-3 prime-phosphate
  modified_base order(4,14): mod_base = OTHER; note = 2 prime-O-methylguanosine-3 prime-phosphate
  modified_base 20: mod_base = OTHER; note = 2 prime-O-methylguanosine-3 prime-phosphorothioate
  modified_base 19: mod_base = OTHER; note = 2 prime-O-methyluridine-3 prime-phosphorothioate
  modified_base order(6,15): mod_base = OTHER; note = 2 prime-O-methylcytidine-3 prime-phosphate
  modified_base order(8,10,12,13,16,17,18,21): mod_base = OTHER; note = 2 prime-O-methyluridine-3 prime-phosphate
  modified_base 7: mod_base = OTHER; note = 2 prime-fluorocytidine-3 prime-phosphate
  modified_base 9: mod_base = OTHER; note = 2 prime-fluoroguanosine-3 prime-phosphate
  modified_base 11: mod_base = OTHER; note = Deoxythymidine-3 prime-phosphate
ctagacctgttttgcttttgt

SEQ ID NO: 4 - RNA, 23 nt, synthetic construct
  source 1..23: mol_type = other RNA
  modified_base order(4,5,6,10,12,14): mod_base = OTHER; note = 2 prime-fluoroadenosine-3 prime-phosphate
  modified_base order(3,9,11,20,23): mod_base = OTHER; note = 2 prime-O-methyladenosine-3 prime-phosphate
  modified_base order(7,15): mod_base = OTHER; note = 2 prime-O-methylguanosine-3 prime-phosphate
  modified_base order(17,19): mod_base = OTHER; note = 2 prime-O-methyluridine-3 prime-phosphate
  modified_base order(8,18): mod_base = OTHER; note = 2 prime-fluorocytidine-3 prime-phosphate
  modified_base 13: mod_base = OTHER; note = 2 prime-O-methylcytidine-3 prime-phosphate
  modified_base 16: mod_base = OTHER; note = 2 prime-fluoroguanosine-3 prime-phosphate
  modified_base 1: mod_base = OTHER; note = 2 prime-O-methyladenosine-3 prime-phosphorothioate
  modified_base 2: mod_base = OTHER; note = 2 prime-fluorocytidine-3 prime-phosphorothioate
  modified_base 21: mod_base = OTHER; note = 2 prime-O-methylguanosine-3 prime-phosphorothioate
  modified_base 22: mod_base = OTHER; note = 2 prime-O-methyladenosine-3 prime-phosphorothioate
acaaaagcaaaacaggtctagaa

SEQ ID NO: 5 - RNA, 23 nt, synthetic construct
  source 1..23: mol_type = other RNA
ttctagacctgttttgcttttgt

SEQ ID NO: 6 - protein, 692 aa, synthetic construct
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

SEQ ID NO: 7 - RNA, 22 nt, synthetic construct
  source 1..22: mol_type = other RNA
  modified_base 1: mod_base = OTHER; note = 2 prime-O-methylcytidine-3 prime-phosphorothioate
  modified_base 2: mod_base = OTHER; note = 2 prime-O-methyluridine-3 prime-phosphorothioate
  modified_base 22: mod_base = OTHER; note = Triantennary N-acetylgalactosamine
  modified_base order(8,10,12,13,16,17,18,19,21): mod_base = OTHER; note = 2 prime-O-methyluridine-3 prime-phosphate
  modified_base order(3,5): mod_base = OTHER; note = 2 prime-O-methyladenosine-3 prime-phosphate
  modified_base order(4,14,20): mod_base = OTHER; note = 2 prime-O-methylguanosine-3 prime-phosphate
  modified_base order(6,15): mod_base = OTHER; note = 2 prime-O-methylcytidine-3 prime-phosphate
  modified_base 7: mod_base = OTHER; note = 2 prime-fluorocytidine-3 prime-phosphate
  modified_base 9: mod_base = OTHER; note = 2 prime-fluoroguanosine-3 prime-phosphate
  modified_base 11: mod_base = OTHER; note = Deoxythymidine-3 prime-phosphate
ctagacctgttttgcttttgtg

SEQ ID NO: 8 - RNA, 24 nt, synthetic construct
  source 1..24: mol_type = other RNA
  modified_base 11: mod_base = OTHER; note = Deoxythymidine-3 prime-phosphate
  modified_base order(8,10,12,13,16,17,18,19,21): mod_base = OTHER; note = 2 prime-O-methyluridine-3 prime-phosphate
  modified_base order(3,5): mod_base = OTHER; note = 2 prime-O-methyladenosine-3 prime-phosphate
  modified_base order(4,14,20): mod_base = OTHER; note = 2 prime-O-methylguanosine-3 prime-phosphate
  modified_base order(6,15): mod_base = OTHER; note = 2 prime-O-methylcytidine-3 prime-phosphate
  modified_base order(22^23,23^24): mod_base = OTHER; note = 3 prime-phosphorothioate
  modified_base 7: mod_base = OTHER; note = 2 prime-fluorocytidine-3 prime-phosphate
  modified_base 9: mod_base = OTHER; note = 2 prime-fluoroguanosine-3 prime-phosphate
  modified_base 1: mod_base = OTHER; note = 2 prime-O-methylcytidine-3 prime-phosphorothioate
  modified_base 2: mod_base = OTHER; note = 2 prime-O-methyluridine-3 prime-phosphorothioate
  modified_base order(22,23,24): mod_base = OTHER; note = A monomer of Formula 2, for example a monomer of formula (C)
ctagacctgttttgcttttgtggg

SEQ ID NO: 9 - RNA, 24 nt, synthetic construct
  source 1..24: mol_type = other RNA
  modified_base order(22^23,23^24): mod_base = OTHER; note = 3 prime-phosphorothioate
  modified_base order(22,23,24): mod_base = OTHER; note = A monomer of Formula 3a, for example a monomer of formula (A)
  modified_base 1: mod_base = OTHER; note = 2 prime-O-methylcytidine-3 prime-phosphorothioate
  modified_base 2: mod_base = OTHER; note = 2 prime-O-methyluridine-3 prime-phosphorothioate
  modified_base order(8,10,12,13,16,17,18,19,21): mod_base = OTHER; note = 2 prime-O-methyluridine-3 prime-phosphate
  modified_base order(3,5): mod_base = OTHER; note = 2 prime-O-methyladenosine-3 prime-phosphate
  modified_base order(4,14,20): mod_base = OTHER; note = 2 prime-O-methylguanosine-3 prime-phosphate
  modified_base order(6,15): mod_base = OTHER; note = 2 prime-O-methylcytidine-3 prime-phosphate
  modified_base 7: mod_base = OTHER; note = 2 prime-fluorocytidine-3 prime-phosphate
  modified_base 9: mod_base = OTHER; note = 2 prime-fluoroguanosine-3 prime-phosphate
  modified_base 11: mod_base = OTHER; note = Deoxythymidine-3 prime-phosphate
ctagacctgttttgcttttgtggg

SEQ ID NO: 10 - RNA, 24 nt, synthetic construct
  source 1..24: mol_type = other RNA
  modified_base order(22^23,23^24): mod_base = OTHER; note = 3 prime-phosphorothioate
  modified_base order(22,23,24): mod_base = OTHER; note = A monomer of Formula 3, for example a monomer of formula (B)
  modified_base 1: mod_base = OTHER; note = 2 prime-O-methylcytidine-3 prime-phosphorothioate
  modified_base 2: mod_base = OTHER; note = 2 prime-O-methyluridine-3 prime-phosphorothioate
  modified_base order(8,10,12,13,16,17,18,19,21): mod_base = OTHER; note = 2 prime-O-methyluridine-3 prime-phosphate
  modified_base order(4,14,20): mod_base = OTHER; note = 2 prime-O-methylguanosine-3 prime-phosphate
  modified_base order(6,15): mod_base = OTHER; note = 2 prime-O-methylcytidine-3 prime-phosphate
  modified_base order(3,5): mod_base = OTHER; note = 2 prime-O-methyladenosine-3 prime-phosphate
  modified_base 7: mod_base = OTHER; note = 2 prime-fluorocytidine-3 prime-phosphate
  modified_base 9: mod_base = OTHER; note = 2 prime-fluoroguanosine-3 prime-phosphate
  modified_base 11: mod_base = OTHER; note = Deoxythymidine-3 prime-phosphate
ctagacctgttttgcttttgtggg

(19) United States
(12) Patent Application Publication
(10) Pub. No.: US 2024/0327842 A1
(43) Pub. Date: Oct. 3, 2024

(54) GALNAC-MONOMERS AND GALNAC-NUCLEIC ACID CONJUGATES

(71) Applicant: LERNA BIOPHARMA PTE. LTD., Amnios (SG)

(72) Inventors: Xi LIN, Amnios (SG); Bing Yuan HAN, Amnios (SG); Pin Koon EE, Amnios (SG); Binxia YANG, Amnios (SG); Joo Leng LOW, Amnios (SG)

(73) Assignee: LERNA BIOPHARMA PTE. LTD., Amnios (SG)

(21) Appl. No.: 18/681,992

(22) PCT Filed: Aug. 8, 2022
(86) PCT No.: PCT/EP2022/072271

§ 371 Date: Feb. 7, 2024

(30) Foreign Application Priority Data
Aug. 9, 2021 (GB) 2111456.6

(51) Int. Cl. C12N 15/113; C07H 15/08; C07H 19/06; C07H 21/02; C07H 21/04

(52) U.S. Cl. CPC C12N 15/113; C07H 15/08; C07H 19/06; C07H 21/02; C07H 21/04; C12N 2310/14; C12N 2310/351

(57) ABSTRACT

GalNAc monomers and GalNAc-oligonucleotide conjugates made using said GalNAc monomers, for example, GalNAc-bearing nucleic acids, e.g. sa-RNAs and siRNAs that may be useful in regulating expression of a target gene.

This application claims priority from GB 2111456.6 filed 9 Aug. 2021, the contents and elements of which are herein incorporated by reference for all purposes.

### TECHNICAL FIELD

The present invention relates to GalNAc monomers. It further relates to GalNAc-oligonucleotide conjugates made using said GalNAc monomers, for example, GalNAc-bearing nucleic acids, e.g. sa-RNAs and siRNAs that may be useful in regulating expression of a target gene.

### BACKGROUND

Oligonucleotides offer great potential as therapeutic agents. While sequences comprising only natural building blocks have found significant utility, innovations to the art of oligonucleotide synthesis mean that oligonucleotides themselves have been subject to a variety of modifications. Chemical modification can improve oligonucleotide activity and/or deliver properties that are not present in naturally occurring oligonucleotides. These approaches generally involve providing modified building blocks that become constituent parts of the oligonucleotide.

Assorted structural motifs are known in the art for the construction of building blocks for derivatized synthetic oligonucleotides. Among these are phosphoramidite reagents, which have been described in numerous publications in the manufacture of RNAs.

For example, patent U.S. Pat. No. 10,781,175 B2 from applicant AM Chemicals LLC describes non-nucleoside solid supports and phosphoramidite building blocks for the preparation of synthetic oligonucleotides containing at least one non-nucleosidic moiety conjugated to a ligand of practical interest. The synthetic oligonucleotides are described to have useful properties such as protein binding, specific receptor binding, and soliciting an immune response.

Patents U.S. Pat. Nos. 8,828,956 B2 and 8,106,022 B2 from applicant Alnylam Pharmaceuticals, Inc. describe RNAi agents comprising carbohydrate conjugates for therapeutic use, in particular targeting the parenchymal cells of the liver or a gene of a hepatitis virus.

Patent U.S. Pat. No. 10,131,907 B2 from applicant Alnylam Pharmaceuticals, Inc. describes RNAi agents comprising at least one galactosyl moiety for inhibiting gene expression activity in cells, particularly hepatocytes, for therapeutic applications.

Patent application WO 2021/032777 A1 from applicant Mina Therapeutics Limited describes GalNAc moieties comprising at least one GalNAc monomer, and GalNAc-oligonucleotide conjugates comprising GalNAc moieties and oligonucleotides such as small activating RNAs (saRNAs) or small inhibiting RNAs (siRNAs). The GalNAc-oligonucleotide conjugates are described as useful in regulating the expression of a target gene.

Hofmeister et al. have described morpholine-GalNAc moieties. The morpholine is substituted with a thymine, and is described as an ASGPR-targeting agent.

Despite these advances, there remains a need in the art for further modified building blocks for the synthesis of therapeutic oligonucleotides. The present invention has been devised with this need in mind.

### SUMMARY

The present invention provides GalNAc monomers suitable for use in the synthesis of oligonucleotides, for example, GalNAc-bearing nucleic acids, e.g. sa-RNAs and siRNAs, as well as GalNAc-oligonucleotide conjugates comprising said monomers. The monomers and conjugates are collectively referred to herein as “compounds”. It will be appreciated therefore that R₁, as defined in the structures, may be a moiety suitable for derivatisation and/or reaction in oligonucleotide synthesis (the monomers) or may be a bond to an oligonucleotide chain.

In a first aspect, the invention may provide a compound of Formula 1, Formula 2, or Formula 3

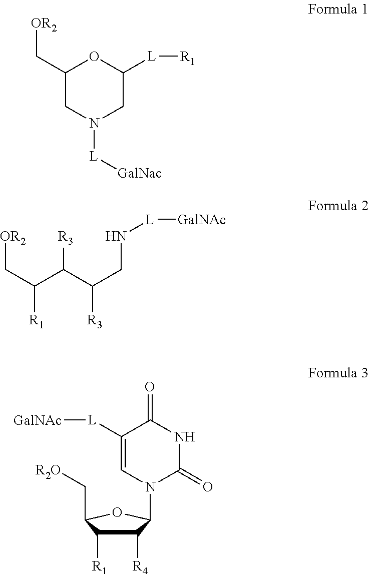

or a pharmaceutically acceptable salt thereof;
wherein
R₁ is O-PN(C₁₋₄alkyl)₂OCH₂CH₂CN, OH, a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage;
R₂ is H or a protecting group;
each R₃ is independently C₁₋₄alkyl, OC₁₋₄alkyl, C₁₋₄haloalkyl, OC₁₋₄haloalkyl or H;
R₄ is H, OH, OC₁₋₄alkyl or halogen;
L is —(W—Y)ₖ—W—X—;
k is 0 to 5;
each W is independently L1 or L2;
each L1 is (CH₂)ₙ, where n is independently 1 to 25;
each L2 is CH₂CH₂(HetCH₂CH₂)ₘ, where m independently is 1 to 24, and Het is independently a heteroatom;
X is a bond, Het, —CH₂—, —CO—, *O—CH₂—CO, *—(Het)CH₂C≡C—, or *—CH₂C≡C—, where * if present denotes the point of attachment to W; and
each Y is independently CONZ, O—CH₂—CONZ, NZCO, SO₂NZ, O—CH₂SO₂NZ or NZSO₂, where Z is H, C₁₋₄alkyl or a protecting group; and wherein
GalNAc may be protected may be deprotected.

In some embodiments, X is a bond, Het, —CH₂—, —CO—, *—(Het)CH₂C≡C—, or *—CH₂C≡C—, where * if present denotes the point of attachment to W; and each Y is independently CONZ, NZCO, SO₂NZ or NZSO₂, where Z is H, C₁₋₄alkyl or a protecting group

In some embodiments where the compound is a compound of Formula 1, Formula 2, or Formula 3,
L is a linker selected from -L1-X—, -L1-Y-L1-X—, -L1-Y-L2-X—, -L2-X—, -L2-Y-L1-X—, or -L2-Y-L2-X-;
L1 is (CH₂)ₙ, where n is 2 to 25;
L2 is CH₂CH₂(HetCH₂CH₂)ₘ, where m is 1 to 12, and Het is a heteroatom;
X is a bond, Het, —CH₂—, —CO—, *—(Het)CH₂C≡C—, or *—CH₂C≡C—; and
Y is CONZ, NZCO, SO₂NZ or NZSO₂, where Z is H, C₁₋₄alkyl or a protecting group.

In some embodiments where the compound is a compound of Formula 1, Formula 2, or Formula 3,
L is a linker selected from -L1-X—, -L1-Y-L1-X—, -L1-Y-L2-X—, -L2-X—, -L2-Y-L1-X—, or -L2-Y-L2-X-;
L1 is (CH₂)ₙ, where n is 2 to 25;
L2 is CH₂CH₂(HetCH₂CH₂)ₘ, where m is 1 to 12, and Het is a heteroatom;
X is a bond, Het, —CH₂—, —CO—, *O—CH₂—CO, *—(Het)CH₂C≡C—, or *—CH₂C≡C—; and
Y is CONZ, O—CH₂—CONZ, NZCO, SO₂NZ, O—CH₂SO₂NZ or NZSO₂, where Z is H, C₁₋₄alkyl or a protecting group.

In some embodiments, the compound is a compound of Formula 1

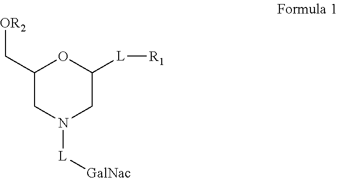

or a pharmaceutically acceptable salt thereof;
wherein
R₁ is O-PN(C₁₋₄alkyl)₂OCH₂CH₂CN, OH, a phosphoramidite linkage to an oligonucleotide or a polystyrene bead or long chain alkylamine controlled-pore glass (LCAA-CPG) which is connected through a succinic, diglycolic or hydroquinone-O,O′diacetic acid linkage;
R₂ is H or a protecting group;
each R₃ is independently C₁₋₄alkyl, OC₁₋₄alkyl, C₁₋₄haloalkyl, OC₁₋₄haloalkyl or H;
R₄ is H, OH, OC₁₋₄alkyl or halogen;
L is —(W—Y)ₖ—W—X—;
k is 0 to 5;
each W is independently L1 or L2;
each L1 is (CH₂)ₙ, where n is independently 1 to 25;
each L2 is CH₂CH₂(HetCH₂CH₂)ₘ, where m independently is 1 to 24, and Het is independently a heteroatom;
X is a bond, Het, —CH2— —CO— or *O—CH₂—CO, for example a bond, Het, —CH₂—, or —CO—; and
each Y is independently CONZ, O—CH₂—CONZ, NZCO, SO₂NZ, O—CH₂SO₂NZ or NZSO₂, for example, CONZ, NZCO, SO₂NZ or NZSO₂, where Z is H, C₁₋₄alkyl or a protecting group;
and wherein GalNAc may be protected may be deprotected.

In some embodiments, the compound is a compound of Formula 2

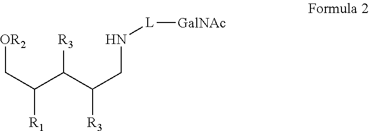

or a pharmaceutically acceptable salt thereof;
wherein
R₁ is O-PN(C₁₋₄alkyl)₂OCH₂CH₂CN, OH, a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage;
R₂ is H or a protecting group;
each R₃ is independently C₁₋₄alkyl, OC₁₋₄alkyl, C₁₋₄haloalkyl, OC₁₋₄haloalkyl or H;
R₄ is H, OH, OC₁₋₄alkyl or halogen;
L is —(W—Y)ₖ—W—X—;
k is 0 to 5;
each W is independently L1 or L2;
each L1 is (CH₂)ₙ, where n is independently 1 to 25;
each L2 is CH₂CH₂(HetCH₂CH₂)ₘ, where m independently is 1 to 24, and Het is independently a heteroatom;
X is a bond, Het, —CH2— —CO— or *O—CH₂—CO, for example a bond, Het, —CH₂—, or —CO—; and
each Y is independently CONZ, O—CH₂—CONZ, NZCO, SO₂NZ, O—CH₂SO₂NZ or NZSO₂, for example, CONZ, NZCO, SO₂NZ or NZSO₂, where Z is H, C₁₋₄alkyl or a protecting group;
and wherein GalNAc may be protected may be deprotected.

In some embodiments, the compound is a compound of Formula 3a

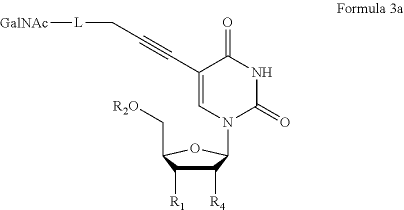

or a pharmaceutically acceptable salt thereof;
wherein
R₁ is O-PN(C₁₋₄alkyl)₂OCH₂CH₂CN, OH, a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage;
R₂ is H or a protecting group;
each R₃ is independently C₁₋₄alkyl, OC₁₋₄alkyl, C₁₋₄haloalkyl, OC₁₋₄haloalkyl or H;

R₄ is H, OH, OC₁₋₄alkyl or halogen;
L is —(W—Y)ₖ—W—X—;
k is 0 to 5;
each W is independently L1 or L2;
each L1 is (CH₂)ₙ, where n is independently 1 to 25;

each L2 is CH₂CH₂(HetCH₂CH₂)ₘ, where m independently is 1 to 24, and Het is independently a heteroatom;
X is a bond, Het, —CH2— —CO— or *O—CH₂—CO, for example a bond, Het, —CH₂—, or —CO—; and
each Y is independently CONZ, O—CH₂—CONZ, NZCO, SO₂NZ, O—CH₂SO₂NZ or NZSO₂, for example, CONZ, NZCO, SO₂NZ or NZSO₂, where Z is H, C₁₋₄alkyl or a protecting group;
and wherein GalNAc may be protected may be deprotected.

In some embodiments where the compound is a compound of Formula 1, Formula 2, or Formula 3a,
L is a linker selected from -L1-X—, -L1-Y-L1-X—, -L1-Y-L2-X—, -L2-X—, -L2-Y-L1-X—, or -L2-Y-L2-X-;
L1 is (CH₂)ₙ, where n is 2 to 25;
L2 is CH₂CH₂(HetCH₂CH₂)ₘ, where m is 1 to 12, and Het is a heteroatom;

X is a bond, Het, —CH2— —CO— or *O—CH₂—CO, for example a bond, Het, —CH₂—, or —CO—; and each Y is independently CONZ, O—CH₂—CONZ, NZCO, SO₂NZ, O—CH₂SO₂NZ or NZSO₂, for example, CONZ, NZCO, SO₂NZ or NZSO₂, where Z is H, C₁₋₄alkyl or a protecting group.

In a further aspect, the invention may provide an oligonucleotide comprising or made using a compound as defined in any preceding claim. Suitably, said oligonucleotide comprises one or more residues derived from said monomers, preferably two or three, more preferably three. In some embodiments said residues are arranged adjacent in the sequence, for example at the 3′ end. The invention includes one or more residues elsewhere in the chain.

In providing the present invention, the inventors were mindful of the following challenges: 1. to improve the coupling efficiency by having phosphoramidite moiety with less hinderance in modified phosphoramidite building blocks, 2. to simplify the manufacturing process by introducing less stereoisomers in precursor synthesis for phosphoramidite building block, 3. to achieve optimal therapeutical effect by tuning the stability of DNA or RNA duplexes with rigid or flexible phosphodiester bonds.

### DESCRIPTION

The compounds described herein are GalNAc monomers and oligonucleotides comprising such monomers. The GalNAc monomers described herein may be useful in the synthesis of oligonucleotides bearing a GalNAc moiety. In other words, they may be used as modified building blocks in the synthesis of oligonucleotides, for example, antisense oligonucleotide (ASOs), small activating RNAs (saRNAs) or small inhibiting RNAS (siRNAs).

The GalNAc monomers described herein may lead to better yields in the synthesis of such oligonucleotides. Additionally or alternatively, the GalNAc monomers described herein may lead to easier manufacturing processes for the synthesis of these oligonucleotides. The structures provided here may lead to optimal therapeutic effect of oligonucleotides with a range of stabilities.

### Compounds

In some cases, the compound is selected from the following formulae:

TABLE 1

| | |
|---|---|
| 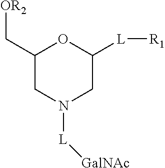 | Formula 1 |
| 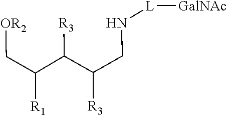 | Formula 2 |
| 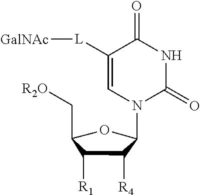 | Formula 3 |
| 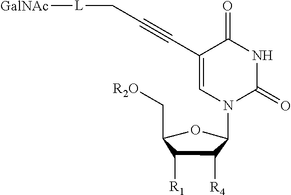 | Formula 3a |

It will be understood that references to compounds include, where appropriate, pharmaceutically acceptable salts, hydrates and solvates thereof.

### GalNAc

GalNAc (IUPAC name N-acetylgalactosamine) is an amino sugar derivative of galactose. It is used as a targeting ligand in antisense and saRNA and siRNA hepatic therapies.

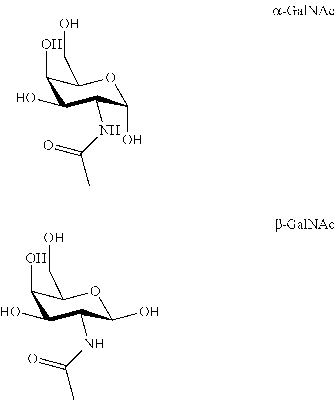

In the monomers and conjugates described herein, the GalNAc is attached via C1. The skilled person will appreciate that, as is conventional in sugar chemistry, a-and B-stereochemistries are possible. Both stereochemistries are envisaged. On some embodiments, the a-stereochemistry is used. In some embodiments, and as exemplified herein, the B-stereochemistry is used.

During the synthesis of oligonucleotides and oligonucleotides conjugates, the GalNAc may be protected, for example with acetyl groups. Accordingly, it will be appreciated that the term GalNAc as used herein refers both to the structure having free hydroxyls as shown and the structure in which these hydroxyls are protected with suitable protecting groups.

That is, GalNAc as written in the formulae described herein may, unless otherwise specified, be a moiety as shown below, where P is hydrogen or a protecting group (for example, acetyl).

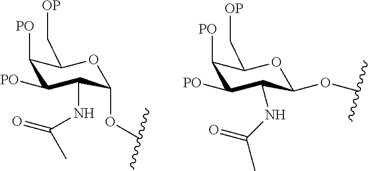

Generally, in structures in which R₁ is O-PN(C₁₋₄alkyl)₂OCH₂CH₂CN, OH, or a polystyrene bead or LCAA-CPG, GalNAc is protected, such that the GalNAc is protected for the reaction to form an oligonucleotide or an oligonucleotide conjugate. For example, the GalNAc may be fully protected with acetyl groups. That is, each P may be a protecting group, for example acetyl.

In structures in which R₁ is a phosphoramidite linkage to an oligonucleotide, the GalNAc may be protected (for example, immediately after synthesis) as described above or may be unprotected, as is normal for final products of this type. That is, each P may be hydrogen.

### The Group R₁

R₁ represents a chemical moiety for attaching the monomer to an oligonucleotide chain, a precursor group, or a point of attachment to an oligonucleotide chain. Phosphoramidite chemistry, as discussed herein, is preferred. Accordingly, in monomer units, R₁ is suitably O-PN(C₁₋₄alkyl)₂OCH₂CH₂CN, wherein said alkyl may be linear or branched, preferably iso-propyl.

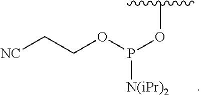

For example, R₁ may be

In some embodiments, R₁ is OH, said free hydroxyl group being suitable for reaction with, for example, a chlorophosphoramidite such as 2-cyanoethyl N,N-diisopropylchlorophosphoramidite.

In some embodiments, R₁ is a phosphoramidite linkage to an oligonucleotide.

In some embodiments, R₁ is a long chain alkylamine controlled-pore glass (LCAA-CPG) which is connected through a succinic, diglycolic or hydroquinone-O,O′diacetic acid linkage.

### Group R₂

Monomers of the invention may include hydroxyl groups which are protected during synthesis steps. It will be appreciated that the invention extends to both these free hydroxyls and protected forms thereof. Accordingly, R₂ may be a protecting group or H.

In some cases, R₂ is selected from Tr, MMTr, DMTr or TMTr protecting groups. Tr is trityl. MMTr is 4′-methoxytrityl. DMTr is dimethoxytrityl (IUPAC name bis-(4-methoxyphenyl)-phenylmethyl). TMTr is 4′, 4′, 4′-trimethoxytrityl. They are protecting groups widely used for protection of the 5′-hydroxy group in nucleosides, particularly in oligonucleotide synthesis. The skilled person will appreciate that other suitable protecting groups may be used.

### Group R₃

Where present, each R₃ may be H or a suitable substituent. Suitably, R₃ is C₁₋₄alkyl, OC₁₋₄alkyl, C₁₋₄haloalkyl, OC₁₋₄haloalkyl or H.

In some embodiments, R₃ is a methyl group, a methoxyl group or H. In some embodiments, R₃ is H.

### Group R₄

Monomers of the invention may include a substituent group which is commonly used in oligonucleotide monomers. This is denoted R₄. Accordingly R₄ may be a substiuent or H.

Suitable R₄ substituents include hydroxyl, OC₁₋₄alkyl (preferably OMe), and halogen (preferably F).

### Linkers

The general formulae include linkers. Collectively, there are referred to herein as L. Linkers may be the same or different. For example, different linkers may be preferred for different formulae described herein, and where more than one linker is present in a formula, those linkers may be the same or different.

Suitably when the compound is a compound of Formula 1, Formula 2, or Formula 3,
L is —(W—Y)ₖ—W—X—;
wherein
k is 0 to 5;
each W is independently L1 or L2;
each L1 is (CH₂)ₙ, where n is 1 to 25;

each L2 is CH₂CH₂(HetCH₂CH₂)ₘ, where m is 1 to 24, and Het is a heteroatom;
X is a bond, a heteroatom (Het), —CH₂—, —CO—, *O—CH₂—CO, *—(Het)CH₂C≡C—, or *—CH₂C≡C—, where * if present denotes the point of attachment to W; and
each Y is independently CONZ, O—CH₂—CONZ, NZCO, SO₂NZ, O—CH₂SO₂NZ or NZSO₂, where Z is H, C₁₋₄alkyl or a protecting group.

In some embodiments,
k is 0 to 5;
each W is independently L1 or L2;
each L1 is (CH₂)ₙ, where n is 1 to 25;
each L2 is CH₂CH₂(HetCH₂CH₂)ₘ, where m is 1 to 24, and Het is a heteroatom;
X is a bond, a heteroatom (Het), —CH₂—, —CO—, *—(Het)CH₂C≡C—, or *—CH₂C≡C—, where * if present denotes the point of attachment to W; and
each Y is independently CONZ, NZCO, SO₂NZ or NZSO₂, where Z is H, C₁₋₄alkyl or a protecting group.

In some embodiments, k is 0 to 2. In some embodiments, k is 0 or 1.

In some embodiments, L is-L1-X—, -L1-Y-L1-X—, -L1-Y-L2-X—, -L2-X—, -L2-Y-L1-X—, -L2-Y-L2-X—, or -L1-Y-L2-Y-L1-X-.

In some embodiments, L is-L1-X—, -L1-Y-L1-X—, -L1-Y-L2-X—, -L2-X—, -L2-Y-L1-X—, or -L2-Y-L2-X-;
wherein
each L1 is (CH₂)ₙ, where n is 2 to 25;
each L2 is CH₂CH₂(HetCH₂CH₂)ₘ, where m is 1 to 12, and Het is a heteroatom;
X is a bond, a heteroatom (Het), —CH₂—, —CO—, *O—CH₂—CO, *—(Het)CH₂C≡C—, or *—CH₂C≡C—; for example, a bond, a heteroatom (Het), —CH₂—, —CO—, *—(Het)CH₂C≡C—, or *—CH₂C≡C—; and
each Y is CONZ, O—CH₂—CONZ, NZCO, SO₂NZ, O—CH₂SO₂NZ or NZSO₂, where Z is H, C₁₋₄alkyl or a protecting group; for example Y is CONZ, NZCO, SO₂NZ or NZSO₂.

In some embodiments, L is-L1-X—, -L1-Y-L1-X—, -L1-Y-L2-X—, -L2-X—, -L2-Y-L1-X—, or -L2-Y-L2-X-;
each L1 is (CH₂)ₙ, where n is 2 to 10;
each L2 is CH₂CH₂(OCH₂CH₂)ₘ, where m is 1 to 5;
X is a bond, —CH₂—, —CO—, *O—CH₂—CO, —O—, *—OCH₂C≡C—, or *—CH₂C≡C—, for example, a bond, —CH₂—, —CO—, —O—, *—OCH₂C≡C—, or *—CH₂C≡C—; and
Y is CONZ or O—CH₂—CONZ, for example O—CH₂—CONZ, where Z is H, C₁₋₄alkyl or a protecting group.

In some embodiments, L is-L1-X—, -L1-Y-L1-X—, -L1-Y-L2-X—, -L2-X—, -L2-Y-L1-X—, or -L2-Y-L2-X-;
each L1 is (CH₂)ₙ, where n is 2 to 10;
each L2 is CH₂CH₂(OCH₂CH₂)ₘ, where m is 1 to 5;
X is a bond, —CO—, *O—CH₂—CO, —O—, *—OCH₂C≡C—, or *—CH₂C≡C—, for example, a bond, —CO—, —O—, *—OCH₂C≡C—, or *—CH₂C≡C—; and
Y is CONZ or O—CH₂—CONZ, for example O—CH₂—CONZ, where Z is H, C₁₋₄alkyl or a protecting group.

Suitably when the compound is a compound of Formula 1, Formula 2, or Formula 3a,
L is —(W—Y)ₖ—W—X—;
wherein
k is 0 to 5;
each W is independently L1 or L2;
each L1 is (CH₂)ₙ, where n is 1 to 25;
each L2 is CH₂CH₂(HetCH₂CH₂)ₘ, where m is 1 to 24, and Het is a heteroatom;
X is a bond, a heteroatom (Het), —CH₂—, *O—CH₂—CO, or —CO—, for example, a bond, a heteroatom (Het), —CH₂—, or —CO—; and
each Y is independently CONZ, O—CH₂—CONZ, NZCO, SO₂NZ, O—CH₂SO₂NZ or NZSO₂, for example, CONZ, NZCO, SO₂NZ or NZSO₂, where Z is H, C₁₋₄alkyl or a protecting group.

In some embodiments, k is 0 to 2. In some embodiments, k is 0 or 1.

In some embodiments, L is-L1-X—, -L1-Y-L1-X—, -L1-Y-L2-X—, -L2-X—, -L2-Y-L1-X—, -L2-Y-L2-X—, or -L1-Y-L2-Y-L1-X-.

In some embodiments, L is-L1-X—, -L1-Y-L1-X—, -L1-Y-L2-X—, -L2-X—, -L2-Y-L1-X—, or -L2-Y-L2-X-;
wherein
each L1 is (CH₂)ₙ, where n is 2 to 25;
each L2 is CH₂CH₂(HetCH₂CH₂)ₘ, where m is 1 to 12, and Het is a heteroatom;
X is a bond, Het, —CH₂—, *O—CH₂—CO, or —CO—, for example, a bond, Het, —CH₂—, or —CO—; and
each Y is CONZ, O—CH₂—CONZ, NZCO, SO₂NZ, O—CH₂SO₂NZ or NZSO₂, for example, CONZ, NZCO, SO₂NZ or NZSO₂, where Z is H, C₁₋₄alkyl or a protecting group.

In some embodiments, L is-L1-X—, -L1-Y-L1-X—, -L1-Y-L2-X—, -L2-X—, -L2-Y-L1-X—, or -L2-Y-L2-X-;
each L1 is (CH₂)ₙ, where n is 2 to 10;
each L2 is CH₂CH₂(OCH₂CH₂)ₘ, where m is 1 to 5;
X is a bond, —CO—,*O—CH₂—CO, or —O—, for example, a bond, —CO—, or —O—; and
Y is CONZ or O—CH₂—CONZ, for example O—CH₂—CONZ, where Z is H, C₁₋₄alkyl or a protecting group.

It will be understood that where the linker is attached to GalNAc, the arrangement is GalNAc-L-. It will be understood that where the linker is attached to R1, the arrangement is R1-L-. That is, the directionality of the linker should be read GalNAc-L1-X—, GalNAc-L1-Y-L1-X—, GalNAc-L1-Y-L2-X—, GalNAc-L2-X-, GalNAc-L2-Y-L1-X—, GalNAc-L2-Y-L2-X—, R1-L1-X—, R1-L1-Y-L1-X—, R1-L1-Y-L2-X—, R1-L2-X—, R1-L2-Y-L1-X-, R1-L2-Y-L2-X—, etc.

In some embodiments, a linker L is selected from -L1-X—, -L1-Y-L1-X—, and-L1-Y-L2-X-.

### Linkers

### L1

L1 is (CH₂)ₙ. That is, where present L1 is an alkylene chain. Optionally, the alkylene chain may be optionally substituted with one or more substituents selected from halogen (for example, F or CI), C₁₋₄alkyl, C₁₋₄haloalkyl, OC₁₋₄alkyl and OC₁₋₄haloalkyl.

n is 1 to 25. That is, the alkylene chain comprises 1 to 25 carbon atoms. In some embodiments, n is 2 to 25. That is, the alkylene chain comprises 2 to 25 carbon atoms. In some embodiments, n is 2 to 10. That is, the alkylene chain comprises 2 to 10 carbon atoms. In some embodiments, n is 2; that is the alkylene chain is ethylene. In some embodiments, n is 3 to 10. In some embodiments, n is 8 to 10. In some embodiments, n is 3 to 6.

In some embodiments, n is 4 to 9. In some embodiments n is 4; that is, the alkylene chain is butylene. In some embodiments n is 5; that is, the alkylene chain is pentylene. In some embodiments n is 6; that is, the alkylene chain is hexylene. In some embodiments n is 7; that is, the alkylene chain is heptylene. In some embodiments n is 8; that is, the alkylene chain is octylene. In some embodiments n is 9; that is, the alkylene chain is nonylene.

### L2

L2 is CH₂CH₂(HetCH₂CH₂)ₘ; that is, where present L2 is a (HetCH₂CH₂) chain, m is 1 to 24, and Het is a heteroatom, such that the linker comprises 1 to 24 repeating (HetCH₂CH₂) units, in addition to leading ethylene. Additional valencies on a heteroatom, where present, may be occupied by H or C₁₋₄alkyl, preferably H. Exemplary heteroatoms include O, S and N (for example, NH).

In some embodiments, m is 1 to 12. In some embodiments, Het is an oxygen atom.

Where m is 1 and Het is an oxygen atom, L2 is —CH₂CH₂OCH₂CH₂13 ; where m is 2 and Het is an oxygen atom, L2 is —CH₂CH₂OCH₂CH₂OCH₂CH₂—, and so on. In some embodiments, m is 1, 2, 3 or 4. In some embodiments, m is 1, 2 or 3. In some embodiments, m is 1 or 2. In some embodiments, m is 2.

### The X Group

In some embodiments, X is —CO—. In other words, the linker is attached via an acyl group. In some embodiments the linker is -L1-CO—, for example pentanoyl (—CO(CH₂)₄—) or decanoyl (—CO(CH₂)₉—).

In some embodiments, X is a bond. In other words, the linker may be attached to the leading CH₂ of L1 or L2, as appropriate. For example, the linker may be -L1-, -L1-Y-L1-, -L1-Y-L2-, -L2-, -L2-Y-L1-, or -L2-Y-L2-. In some embodiments the linker is -L1-; for example ethylene.

In some embodiments, X is a heteroatom (Het). Additional valencies on a heteroatom, where present, may be occupied by H or C₁₋₄alkyl, preferably H. Exemplary heteroatoms include O, S and N (for example, NH). In some preferred embodiments, X is —O—.

In some embodiments, X is *—(Het)CH₂C≡C—, where * is the point of attachment to W. Additional valencies on a heteroatom, where present, may be occupied by H or C₁₋₄alkyl, preferably H. Exemplary heteroatoms include O, S and N (for example, NH). In some preferred embodiments, X is *—OCH₂C≡C—.

In some embodiments, X is *—CH₂C≡C—, where * is the point of attachment to W. In other words, the linker is attached via a propargylene group. In some embodiments the linker is -L1-CH₂C≡C—.

In some embodiments, X is —CH₂—.

In some embodiments, X is *O—CH₂—CO. It will be appreciated that in units of formula L2-X, X is *O—CH₂—CO may be appropriate as the synthetic route may include oxidising the terminal alcohol of a PEG chain.

The Y group

Each Y is independently CONZ, O—CH₂—CONZ, NZCO, SO₂NZ, O—CH₂SO₂NZ or NZSO₂, where Z is H, C₁₋₄alkyl or a protecting group. In some embodiments, Y is CONZ or SO₂NZ, where Z is H, C₁₋₄alkyl (for example, methyl) or a protecting group.

Similarly, it will be appreciated that in units of formula L2-Y, Y is O—CH₂—CONZ or O—CH₂SO₂NZ may be suitable as the synthetic route may include oxidising the terminal alcohol of a PEG chain.

In some embodiments where more than one Y is present, each Y is the same. In some embodiments, Y is CONZ; in other words, Y provides an amide linkage which may be optionally protected. Suitable nitrogen protecting groups are known in the art and include benzyl (Bn). In some embodiments, Z is H, Bn or C₁₋₄alkyl.

In some embodiments, Y is CONH. That is, the linker is selected from -L1-CONH-L1-X-, -L1-CONH-L2-X—, -L2-CONH-L1-X—, and-L2-CONH-L2-X-; preferably-L1-CONH-L1-X-and-L1-CONH-L2-X-; more preferably-L1-CONH-L2-X-.

In one preferred embodiment, the linker is —(CH₂)₄—CONH—CH₂CH₂OCH₂CH₂13 ; that is the linker is -L1-Y-L2-X—, L1 is present and n is 4, Y is CONH, L2 is present and m is 1, and X is a bond.

In one preferred embodiment, the linker is —(CH₂)₅—CONH—(CH₂)₄—CO—; that is the linker is -L1-Y-L1-X-, where X is—CO—, Y is CONH, both L1 chains are present, and one n is 4 and the other n is 5.

In one preferred embodiment, the linker is —(CH₂)₄—CONH—CH₂CH₂—(OCH₂CH₂)₂—CONH—(CH₂)₅—; that is the linker is -L1-Y-L2-Y-L1-X—, where X is a bond, each Y is CONH, the first L1 has n is 4, the second L1 has n is 5, and m is 2.

In one preferred embodiment, the linker is —(CH₂)₄—CONH—CH₂CH₂(OCH₂CH₂)—O—(CH₂)C≡C—; that is the linker is -L1-Y-L2-X—, L1 is present and n is 4, Y is CONH, L2 is present and m is 1, and the X is *—O(CH₂)C≡C—.

Some preferred linkers

In some embodiments, the linker L is -L1-Y-L1-X— (such that k is 1 and both W are L1), where X is —CO—, Y is CONH, one n is 4 and the other n is 5. That is, the linker is —(CH₂)₄—CONH—(CH₂)₅—CO—. [Linker 1]

In some embodiments, the linker L is -L1-Y-L2-X— (such that k is 1, one W is L1 and the other W is L2), where n is 4, Y is CONH, m is 4, Het is O, and X is —CO—. That is, the linker is —(CH₂)₄—CONH—CH₂CH₂(OCH₂CH₂)₄—CO—. [Linker 2]

In some embodiments, the linker L is -L1-X— (such that k is 0, and W is L1), where n is 9, and X is —CO—. That is, the linker is —(CH₂)₉—CO—. [Linker 3]

In some embodiments, the linker L is -L1-Y-L2-X— (such that k is 1, one W is L1 and the other W is L2), where n is 4, Y is CONH, m is 1, Het is O, and X is —O—CH₂—CO. That is, the linker is —(CH₂)₄—CONH—CH₂CH₂(OCH₂CH₂)—O—CH₂—CO—. [Linker 4]

In some embodiments, the linker L is -L2-X— (such that k is 0, and W is L2), where m is 3, Het is O, and X is —O—CH2—CO—. That is, the linker is —CH₂CH₂(OCH₂CH₂)₃—O—CH₂—CO—. [Linker 5]

In some embodiments, the linker L is -L2-Y-L1-X— (such that k is 1, and one W is L2 and the other W is L1), where m is 1, Het is O, Y is O—CH₂—CONH (that is, Z is H), n is 5, and X is —CO—. That is, the linker is —CH₂CH₂(OCH₂CH₂)-O—CH₂—CONH—(CH₂)₅—CO—. [Linker 6]

In some embodiments, the linker L is -(L1-Y)₂-L1-X— (such that k is 2, and all three W are L1), where one n is 4, another n is 3 and the final n is 1, one Y is CONH (that is, Z is H), and the other Y is NHCO (that is, Z is H), and X is —CH₂C≡C—. That is, the linker is —(CH₂)₄—CONH—(CH₂)₃—NHCO—CH₂—CH₂C≡C—. [Linker 7]

In some embodiments, the linker L is -(L1-Y)₂-L1-X— (such that k is 2, and all three W are L1), where one n is 4, another n is 3 and the final n is 4, one Y is CONH (that is, Z is H), and the other Y is NHCO (that is, Z is H), and X is a bond. That is, the linker is —(CH₂)₄—CONH—(CH₂)₃—NHCO—(CH₂)₄—. [Linker 7—hydrogenated].

In some embodiments, the linker L is -L2-Y-L2-X— (such that k is 1, and both W are L2), where one m is 1, and the other m is 1, both Het are O, Y is O—CH₂—CONH (that is, Z is H), and X is —OCH₂C≡C—(that is, Het is O). That is, the linker is —CH₂CH₂(OCH₂CH₂)—O—CH₂—CONH—CH₂CH₂(OCH₂CH₂)—OCH₂C≡C—. [Linker 8].

In some embodiments, the linker L is -L2-Y-L2-X— (such that k is 1, and both W are L2), where one m is 1, and the other m is 2, both Het are O, Y is O—CH₂—CONH (that is, Z is H), and X is —CH₂—. That is, the linker is —CH₂CH₂(OCH₂CH₂)—O—CH₂—CONH—CH₂CH₂(OCH₂CH₂) 2—CH₂—. [Linker 8—hydrogenated]

In some embodiments, the linker L is -L2-Y-L1-Y-L1-X— (such that k is 2, and one W is L2 and the other two W are L1), where m is 1, one Y is O—CH₂—CONH (that is, Z is H), and one Y is NHCO (that is, Z is H), one n is 3, and the other n is 1, and X is —CH₂C≡C—. That is, the linker is —CH₂CH₂(OCH₂CH₂)—O—CH₂—CONH—(CH₂)₃—NHCO—CH₂—CH₂C≡C—. [Linker 9]

In some embodiments, the linker L is -L2-Y-L1-Y-L1-X— (such that k is 2, and one W is L2 and the other two W are L1), where m is 1, one Y is O—CH₂—CONH (that is, Z is H), and one Y is NHCO (that is, Z is H), one n is 3, and the other n is 4, and X is a bond. That is, the linker is —CH₂CH₂(OCH₂CH₂)—O—CH₂—CONH—(CH₂)₃—NHCO—(CH₂)₄—. [Linker 9—hydrogenated]

In some embodiments, the linker L is -L1-Y-L2-X— (such that k is 1, and one W is L1 and the other W is L2), where n is 4, Y is CONH (that is, Z is H), m is 1, and X is —OCH₂C≡C—(that is, Het is O). That is, the linker is —(CH₂)₄—CONH—CH₂CH₂(OCH₂CH₂)—OCH₂C≡C—. [Linker 10]

In some embodiments, the linker L is -L1-Y-L2-X— (such that k is 1, and one W is L1 and the other W is L2), where n is 4, Y is CONH (that is, Z is H), m is 2, and X is —CH₂—. That is, the linker is —(CH₂)₄—CONH—CH₂CH₂(OCH₂CH₂) 2—CH₂—. [Linker 10—hydrogenated]

### Formula 1

In some embodiments, the compound is a compound of Formula 1. Suitably, the two linkers are different and may be distinguished as L^{a} and L^{b}, as shown in Formula 1a. In some embodiments, the stereochemistry is as shown in Formula 1b.

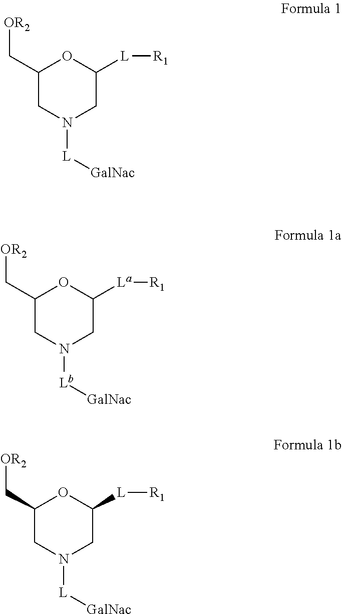

Preferably, L^{a} is a linker of formula-L1-X—, optionally wherein n is 2 and X is an oxygen atom. That is, L^{a} is -(ethylene)O—, and the substituent is R₁-(ethylene)O—.

That is, the compound may be a compound of the following formula:

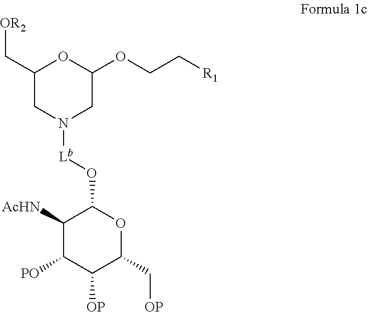

In some alternative embodiments, L^{a} is L2, optionally where m is 1 or 2, preferably 1.

Preferably, L^{b} is a linker of formula -L1-X—, optionally wherein n is 4 and X is —CO—. That is, L^{b} is -(butylene)CO—.

That is, the compound may be a compound of the following formula:

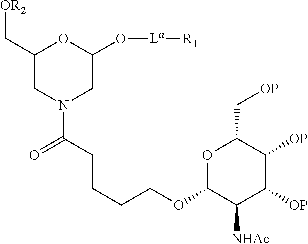

In some embodiments, the invention provides a monomer of the following formula (GalNAc Monomer 1):

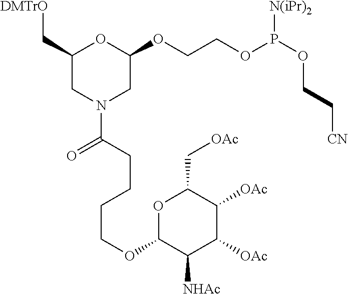

That is, R₁ is O-PN(iPr)₂OCH₂CH₂CN; R₂ is DMTr; a first linker (L^{a}) is -L1-X—, where n=2 and X is an oxygen atom; and the second linker (L^{b}) is -L1-X—, where X is —CO— and n is 4; and GalNAc is protected with acetyl groups (that is, P is Ac).

### Formula 2

In some embodiments, the compound is a compound of Formula 2.

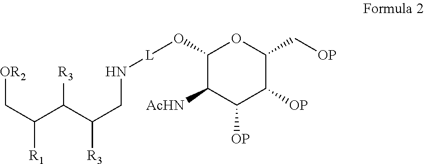

Preferably, each R₃ is independently a methyl group, a methoxyl group or H. More preferably, R₃ is H.

In some embodiments, the compound is a compound of Formula 2a or 2b:

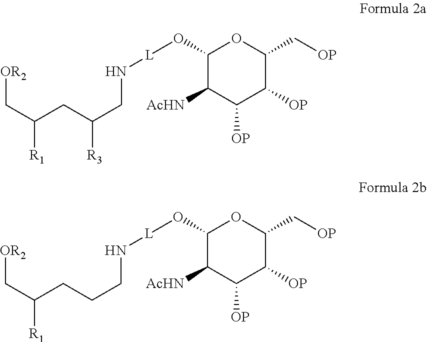

Preferably, the linker is -L1-X—, where X is —CO—. That is, the linker is attached to the amine of the core motif by an amide bond. In some embodiments, n is 8 to 10. In some embodiments n is 9; that is, the linker is decanoyl.

In some embodiments, the linker is -L1-Y-L1-X—, where X is —CO— and Y is CONZ. That is, the linker is attached to the amine of the core motif by an amide bond. In some embodiments, the L1 groups may have different values for n. In some embodiments, each n is independently 4 or 5. In some embodiments, the linker is —(CH₂)₅-Y-(CH₂)₄-X—, for example, —(CH₂)₅—CONH—(CH₂) 4—CO—.

In some embodiments, the invention provides a monomer of the following formula (GalNAc Monomer 2):

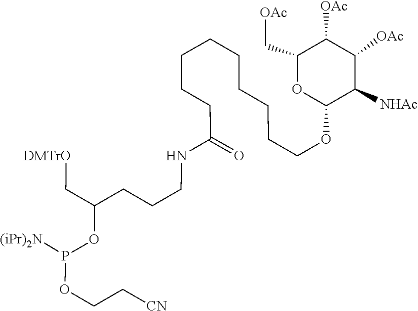

That is, R₁ is O-PN(iPr)₂OCH₂CH₂CN; R₂ is DMTr; both R₃ are H; the linker is -L1-X—, where X is —CO— and n is 9, and GalNAc is protected with acetyl groups (that is, P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage; R₂ is OH, DMTr); both R₃ are H; the linker L is -L1-Y-L1-X— (such that k is 1 and both W are L1), where X is —CO—, Y is CONH, one n is 4 and the other n is 5 (that is, the linker is —(CH₂)₄—CONH—(CH₂)₅—CO—) [Linker 1], and GalNAc is protected with acetyl groups (that is P is Ac).

In some embodiments, the invention provides a monomer of the following formula (GalNAc Monomer 4):

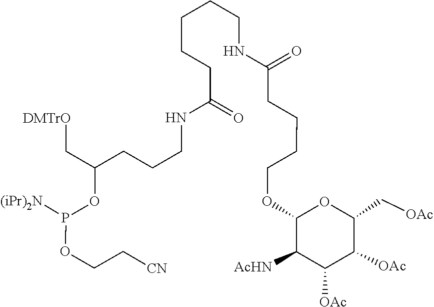

That is, R₁ is O-PN(iPr)₂OCH₂CH₂CN; R₂ is DMTr; both R₃ are H; the linker is -L1-Y-L1-X—, where X is —CO—, Y is CONH, one n is 4 and the other n is 5 (that is, the linker is —(CH₂)₅—CONH—(CH₂)₄—CO—), and GalNAc is protected with acetyl groups (that is, P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage; R₂ is OH or DMTr; both R₃ are H; the linker L is -L1-Y-L2-X— (such that k is 1, one W is L1 and the other W is L2), where n is 4, Y is CONH, m is 4, Het is O, X is —CO— (that is, the linker is —(CH₂)₄—CONH—CH₂CH₂(OCH₂CH₂)₄—CO— [Linker 2]), and GalNAc is protected with acetyl groups (that is P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage; R₂ is OH or DMTr; both R₃ are H; the linker L is -L1-X— (such that k is 0, and W is L1), where n is 9, X is —CO— (that is, the linker is —(CH₂)₉—CO— [Linker 3]), and GalNAc is protected with acetyl groups (that is P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage;

R₂ is OH or DMTr; both R₃ are H; the linker L is -L1-Y-L2-X— (such that k is 1, one W is L1 and the other W is L2), where n is 4, Y is CONH, m is 1, Het is O, X is —O—CH₂—CO (that is, the linker is —(CH₂)₄—CONH—CH₂CH₂(OCH₂CH₂)—O—CH₂—CO— [Linker 4]), and GalNAc is protected with acetyl groups (that is P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage; R₂ is OH or DMTr; both R₃ are H; the linker L is -L2-X— (such that k is 0, and W is L2), where m is 3, Het is O, X is O—CH₂—CO (that is, the linker is —CH₂CH₂(OCH₂CH₂) 3-O—CH₂—CO— [Linker 5]), and GalNAc is protected with acetyl groups (that is P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage; R₂ is OH or DMTr; both R₃ are H; the linker L is -L2-Y-L1-X— (such that k is 1, and one W is L2 and the other W is L1), where m is 1, Het is O, Y is O—CH₂—CONH (that is, Z is H), n is 5, X is —CO— (that is, the linker is —CH₂CH₂(OCH₂CH₂)—O—CH₂—CONH—(CH₂)₅—CO— [Linker 6]), and GalNAc is protected with acetyl groups (that is P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage; R₂ is OH or DMTr; both R₃ are H; the linker L is -(L1-Y)₂-L1-X— (such that k is 2, and all three W is L1), where one n is 4, another n is 3 and the final n is 1, one Y is CONH (that is, Z is H), and the other Y is NHCO (that is, Z is H), and X is —CH₂C≡C— (that is, the linker is —(CH₂)₄—CONH—(CH₂)₃—NHCO—CH₂—CH₂C≡C— [Linker 7]), and GalNAc is protected with acetyl groups (that is P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage; R₂ is OH or DMTr; both R₃ are H; the linker L is -(L1-Y)₂-L1-X— (such that k is 2, and all three W are L1), where one n is 4, another n is 3 and the final n is 4, one Y is CONH (that is, Z is H), and the other Y is NHCO (that is, Z is H), and X is a bond (that is, the linker is —(CH₂)₄—CONH—(CH₂)₃—NHCO—(CH₂)₄— [Linker 7-hydrogenated]), and GalNAc is protected with acetyl groups (that is P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage; R₂ is OH or DMTr; both R₃ are H; the linker L is -L2-Y-L2-X— (such that k is 1, and both W are L2), where one m is 1, and the other m is 1, both Het are O, Y is O—CH₂—CONH (that is, Z is H), and X is —OCH₂C≡C—(that is, Het is O) (that is, the linker is —CH₂CH₂(OCH₂CH₂)—O—CH₂—CONH—CH₂CH₂(OCH₂CH₂)—OCH₂C≡C— [Linker 8]), and GalNAc is protected with acetyl groups (that is P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage; R₂ is OH or DMTr; both R₃ are H; the linker L is -L2-Y-L2-X— (such that k is 1, and both W are L2), where one m is 1, and the other m is 2, both Het are O, Y is O—CH₂—CONH (that is, Z is H), and X is —CH₂— (that is, the linker is —CH₂CH₂(OCH₂CH₂)—O—CH₂—CONH—CH₂CH₂(OCH₂CH₂)₂—CH₂— [Linker 8-hydrogenated), and GalNAc is protected with acetyl groups (that is P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage; R₂ is OH or DMTr; both R₃ are H; the linker L is -L2-Y-L1-Y-L1-X— (such that k is 2, and one W is L2 and the other two W are L1), where m is 1, one Y is O—CH₂—CONH (that is, Z is H), and one Y is NHCO (that is, Z is H), one n is 3, and the other n is 1, and X is —CH₂C≡C— (that is, the linker is —CH₂CH₂(OCH₂CH₂)—O—CH₂—CONH—(CH₂)₃—NHCO—CH₂—CH₂≡C— [Linker 9]), and GalNAc is protected with acetyl groups (that is P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage; R₂ is OH or DMTr; both R₃ are H; the linker L is -L2-Y-L1-Y-L1-X— (such that k is 2, and one W is L2 and the other two W are L1), where m is 1, one Y is O—CH₂—CONH (that is, Z is H), and one Y is NHCO (that is, Z is H), one n is 3, and the other n is 4, and X is a bond (that is, the linker is —CH₂CH₂(OCH₂CH₂)—O—CH₂—CONH—(CH₂)₃—NHCO-(CH₂)₄-[Linker 9-hydrogenated), and GalNAc is protected with acetyl groups (that is P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage; R₂ is OH or DMTr; both R₃ are H; the linker L is -L1-Y-L2-X— (such that k is 1, and one W is L1 and the other W is L2), where n is 4, Y is CONH (that is, Z is H), m is 1, and X is —OCH₂C≡C—(that is, Het is O) (that is, the linker is —(CH₂)₄—CONH—CH₂CH₂(OCH₂CH₂)—OCH₂C≡C— [Linker 10]), and GalNAc is protected with acetyl groups (that is P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage; R₂ is OH or DMTr; both R₃ are H; the linker L is -L1-Y-L2-X— (such that k is 1, and one W is L1 and the other W is L2), where n is 4, Y is CONH (that is, Z is H), m is 2, and X is —CH₂— (that is, the linker is —(CH₂)₄—CONH—CH₂CH₂(OCH₂CH₂)₂—CH₂—), and GalNAc is protected with acetyl groups (that is P is Ac).

### Formula 3

In some embodiments, the compound is a compound of Formula 3.

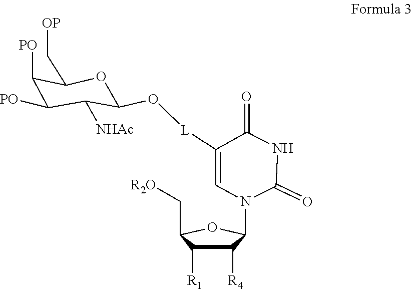

Preferably, R₄ is H, OH, OC₁₋₄alkyl or halogen. More preferably, R₄ is H.

Preferably, X is *—OCH₂C≡C—, where * denotes the point of attachment to W. In some embodiments, the linker is -L1-Y-L2-X—, such as-L1-Y-L2—OCH₂C≡C—(that is, X is *—OCH₂C≡C—). In some embodiments, Y is CONH. In some embodiments, n is 4, Y is CONH, and m is 1.

In some embodiments, the compound is a compound of Formula 3a

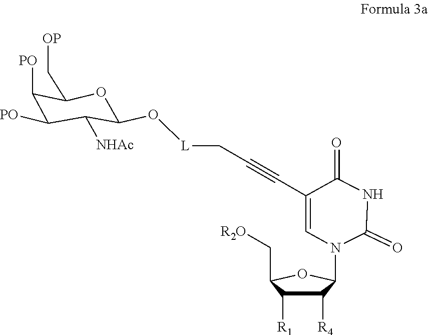

Preferably, X is an oxygen atom. In some embodiments, the linker is -L1-Y-L2-X—, such as-L1-Y-L2-O-(that is, X is an oxygen atom). In some embodiments, Y is CONH. In some embodiments, n is 4, Y is CONH, and m is 1.

In some embodiments, the linker is —(CH₂)₄—CONH—(CH₂)₅—CO— [Linker 1]

In some embodiments, the linker is —(CH₂)₄—CONH—CH₂CH₂(OCH₂CH₂)₄—CO— [Linker 2]

In some embodiments, the linker is —(CH₂)₉—CO— [Linker 3]

In some embodiments, the linker is —(CH₂)₄—CONH—CH₂CH₂(OCH₂CH₂)—O—CH₂—CO— [Linker 4]

In some embodiments, the linker is —CH₂CH₂(OCH₂CH₂)₃—O—CH₂—CO— [Linker 5]

In some embodiments, the linker is —CH₂CH₂(OCH₂CH₂)—O—CH₂—CONH—(CH₂)₅—CO— [Linker 6]

In some embodiments, the linker is —(CH₂)₄—CONH—(CH₂)₃—NHCO—CH₂—CH₂C≡C— [Linker 7]

In some embodiments, the linker is —(CH₂)₄—CONH—(CH₂)₃—NHCO—(CH₂)₄—[Linker 7—hydrogenated]

In some embodiments, the linker is —CH₂CH₂(OCH₂CH₂)—O—CH₂—CONH—CH₂CH₂(OCH₂CH₂)—OCH₂C≡C— [Linker 8]

In some embodiments, the linker is —CH₂CH₂(OCH₂CH₂)—O—CH₂—CONH—CH₂CH₂(OCH₂CH₂)₂—CH₂— [Linker 8—hydrogenated]

In some embodiments, the linker is —CH₂CH₂(OCH₂CH₂)—O—CH₂—CONH—(CH₂)₃—NHCO-CH₂—CH₂C≡C— [Linker 9]

In some embodiments, the linker is —CH₂CH₂(OCH₂CH₂)—O—CH₂—CONH—(CH₂)₃—NHCO-(CH₂)₄— [Linker 9—hydrogenated]

In some embodiments, the linker is —(CH₂)₄—CONH—CH₂CH₂(OCH₂CH₂)—OCH₂C≡C— [Linker 10]

In some embodiments, the linker is —(CH₂)₄—CONH—CH₂CH₂(OCH₂CH₂)₂—CH₂— [Linker 10—hydrogenated]

In some embodiments, the invention provides a monomer of the following formula (GalNAc Monomer 3):

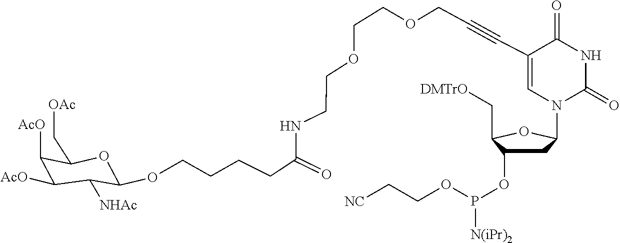

That is in Formula 3, R₁ is O-PN(iPr)₂OCH₂CH₂CN; R₂ is DMTr; R₄ is H; the linker L is -L1-Y-L2-X—, where n is 4, Y is CONH, m is 1 and X is *—OCH₂C≡C—, and GalNAc is protected with acetyl groups (that is, P is Ac). That is in Formula 3a, R₁ is O-PN(iPr)₂OCH₂CH₂CN; R₂ is DMTr; R₄ is H; the linker L is -L1-Y-L2-X—, where n is 4, Y is CONH, m is 1 and X is an oxygen atom, and GalNAc is protected with acetyl groups (that is, P is Ac).

In some embodiments, the invention provides a monomer of the following formula (GalNAc Monomer 5):

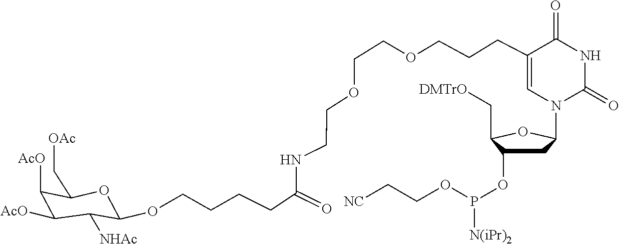

That is in Formula 3, R₁ is O-PN(iPr)₂OCH₂CH₂CN; R₂ is DMTr; R₄ is H; the linker L is -L1-Y-L2-X—, where n is 4, Y is CONH, m is 2 and X is —CH₂—, and GalNAc is protected with acetyl groups (that is, P is Ac). That is, L2 is —CH₂CH₂OCH₂CH₂OCH₂CH₂—.

### Inhibitory Nucleic Acids

Aspects and embodiments of the present disclosure relate to inhibitory nucleic acids. As used herein, an ‘inhibitory nucleic acid’ refers to a nucleic acid capable of reducing or preventing the gene and/or protein expression of one or more given target gene(s)/protein(s). The term ‘monomer residue’ refers to a monomer unit bound within the oligonucleotide chain at one or more positions. In the structure below, the wavy lines denote points of attachment within an oligonucleotide chain or a terminus of an oligonucleotide chain if appropriate.

Accordingly, an oligonucleotide comprising at least one GalNAc monomer residue according to the invention may be an inhibitory nucleic acid.

In other embodiments, an oligonucleotide comprising at least one GalNAc monomer residue according to the invention may be a small activating RNA (saRNA).

Suitably, the oligonucleotide may comprise adjacent GalNAc monomer residues. In some embodiments, the oligonucleotide comprises preferably two, or more preferably three, adjacent monomer residues. It will be appreciated that said monomer residue(s) may be located at any point within the chain. In some preferred embodiments, said monomer residue(s) are located at or near the 3′ end of the oligonucleotide. In some emboidments, said monomer residue(s) are located at or near the 5′ end of the oligonucleotide.

In some embodiments, the disclosure provides an oligonucleotide (e.g. inhibitory nucleic acid) comprising at least one monomer residue of formula:

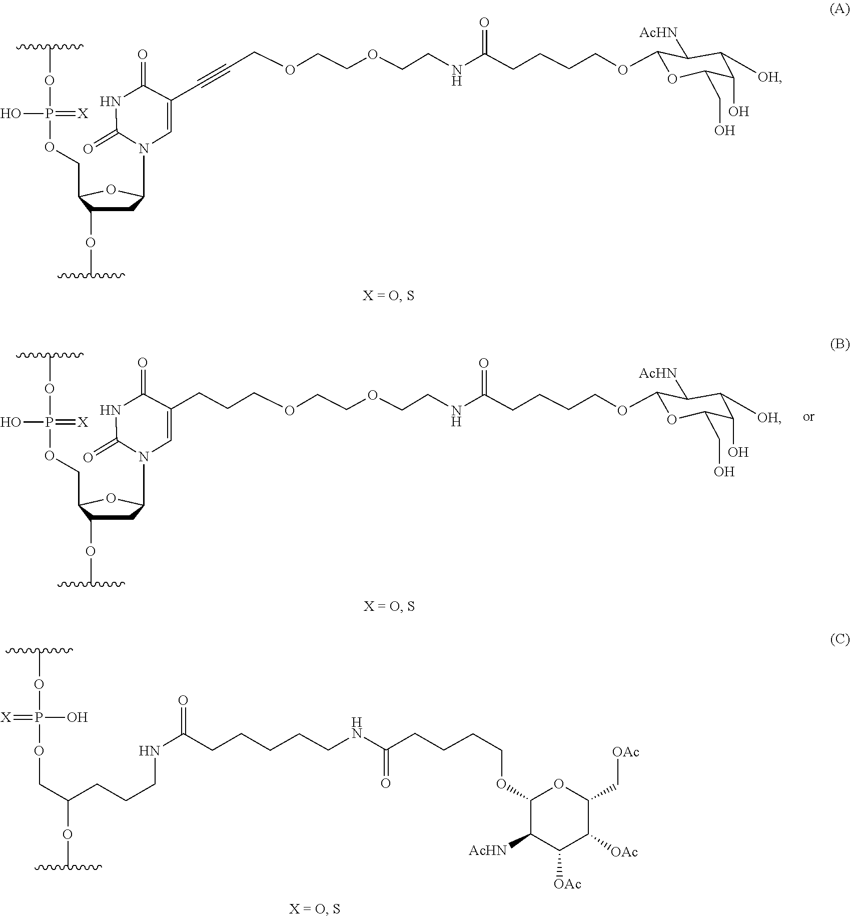

optionally comprising three copies of said monomer residue; that is, three copies of a monomer of formula (A), (B) or (C). Suitably, said copies are successive. In other words, the oligonucleotide comprises three adjacent monomer residues, said monomer residues being selected from one of formula (A), (B) or (C). In some embodiments, X is O. In some embodiments, X is S. Oligonucleotides and siRNAs exemplified herein comprise three monomer residues of formula (A) wherein X is O, X is S and X is S, respectively.

Other oligonucleotides and siRNAs exemplified herein comprise three monomer residues of formula (B) wherein X is O, X is S and X is S, respectively. Yet other oligonucleotides and siRNAs exemplified herein comprise three monomer residues of formula (C) wherein X is O, X is S and X is S, respectively. Said monomer residue(s) may be located at the 3′ end of the oligonucleotide. Alternatively, said monomer residue(s) may be located at the 5′ end of the oligonucleotide or at another point in the chain.

Inhibitory nucleic acids according to the present disclosure may comprise or consist of DNA and/or RNA and/or other types of oligonucleotides. Inhibitory nucleic acids may comprise alterations to oligonucleotide sugar-phosphate backbones in order to reduce/prevent RNAse H degradation, such as e.g. phosphorothioate linkages, phosphorodiamidate linkages such as phosphorodiamidate morpholino (PMOs), and may comprise e.g. peptide nucleic acids (PNAS), locked nucleic acids (LNAs), methoxyethyl nucleotide modifications, e.g. 2′O-methyl (2′OMe) and 2′-O-40 methoxyethyl (MOE) ribose modifications and/or 5′-methylcytosine modifications.

Inhibitory nucleic acids may be single-stranded (e.g. in the case of antisense oligonucleotides). Inhibitory nucleic acids may be double-stranded or may comprise double-stranded region(s) (e.g. in the case of siRNA, shRNA, etc.). Inhibitory nucleic acids may comprise both double-stranded and single-stranded regions (e.g. in the case of shRNA and pre-miRNA molecules, which are double-stranded in the stem region of the hairpin structure, and single-stranded in the loop region of the hairpin structure).

In some embodiments, an inhibitory nucleic acid is a small interfering RNA (SIRNA). As used herein, ‘siRNA’ refers to a double-stranded RNA molecule having a length between 17 to 30 (e.g. 20 to 27, e.g. ˜21) base pairs, which is capable of engaging the RNA interference (RNAi) pathway for the targeted degradation of target RNA. Double-stranded siRNA molecules may be formed as a nucleic acid complex of RNA strands having a high degree of complementarity. The strand of the double-stranded siRNA molecule having complementarity to a target nucleotide sequence (i.e. the antisense nucleic acid) may be referred to as the ‘guide’ strand, and the other strand (sense strand) may be referred to as the ‘passenger’ strand. The structure and function of siRNAs is described e.g. in Kim and Rossi, Biotechniques. 2008 April; 44 (5): 613-616. In some embodiments, siRNA molecules comprise asymmetric 3′ overhangs on the guide strand, e.g. comprising one or two nucleotides (e.g. a ‘UU’ 3′ overhang).

In one aspect, inhibitory nucleic acids according to the present disclosure are suitable for reducing gene and/or protein expression of PCSK9. It will be appreciated that where an inhibitory nucleic acid is described as reducing gene expression of PCSK9, inhibition of expression of the gene encoding PCSK9 is intended. That is, reference herein to inhibition of gene expression of PCSK9 contemplates inhibition of expression of PCSK9.

PCSK9 stands for proprotein convertase subtilisin/kexin type 9.

The structure and function of PCSK9 is described in e.g. in Sobati et al., Adv Pharm Bull. (2020) 10 (4): 502-511 and Urban et al., J Am Coll Cardiol. (2013) 62 (16): 1401-1408, both of which are hereby incorporated by reference in their entirety.

In this specification, reference to ‘PCSK9’ encompasses: human PCSK9 isoform 1, homologues of human PCSK9 isoform 1 (i.e. encoded by the genome of a non-human animal), and variants thereof. In some embodiments, PCSK9 according to the present disclosure comprises or consists of an amino acid sequence having at 70% or greater amino acid sequence identity, preferably one of 75%, 80%, 85%, 90%, 91%, 92%, 93%, 94%, 95%, 96%, 97%, 98%, 99% or 100% amino acid sequence identity to the amino acid sequence of SEQ ID NO: 6.

In some embodiments, an inhibitory nucleic acid according to the present disclosure is an siRNA for reducing gene and/or protein expression of PCSK9 (i.e. siRNA against PCSK9). In some embodiments, an inhibitory nucleic acid according to the present disclosure comprises the nucleotide sequence of inclisiran. Inclisiran is a synthetic siRNA directed against PCSK9, and is described e.g. in Fitzgerald et al., NEJM (2017) 376 (1): 41-51 and WO 2014/089313 A1 (both of which are hereby incorporated by reference in their entirety). The target nucleotide sequence for inclisiran is shown in SEQ ID NO: 5. The nucleotide sequence of the antisense nucleic acid of inclisiran (i.e. the nucleotide sequence of the guide strand) is shown in SEQ ID NO: 2, and the nucleotide sequence of the sense strand (i.e. the nucleotide sequence of the passenger strand) is shown in SEQ ID NO: 1. The guide and/or passenger strand of inclisiran comprise modifications as shown in SEQ ID NO: 4 and 3, respectively.

Accordingly, in some aspects and embodiments of the present disclosure, the inhibitory nucleic acid comprises the nucleotide sequence set forth in SEQ ID NO: 2 and/or the nucleotide sequence set forth in SEQ ID NO: 1. Suitably, in some aspects and embodiments of the present disclosure, the inhibitory nucleic acid comprises the nucleotide sequence set forth in SEQ ID NO: 4 and/or the nucleotide sequence set forth in SEQ ID NO: 3.

In some embodiments, the inhibitory nucleic acid comprises the nucleotide sequence set forth in SEQ ID NO: 8, and preferably further comprises the nucleotide sequence set forth in SEQ ID NO: 4. In some embodiments, the inhibitory nucleic acid comprises the nucleotide sequence set forth in SEQ ID NO: 9, and preferably further comprises the nucleotide sequence set forth in SEQ ID NO: 4. In some embodiments, the inhibitory nucleic acid comprises the nucleotide sequence set forth in SEQ ID NO: 10, and preferably further comprises the nucleotide sequence set forth in SEQ ID NO: 4.

PCSK9 is expressed by hepatocytes, renal mesenchymal cells, intestinal ileum, and colon epithelial cells and telencephalon neurons in the embryonic brain. PCSK9 has been shown to have a central role in regulation of cholesterol homeostasis, by enhancing the endosomal and lysosomal degradation of hepatic low-density lipoprotein receptor (LDLR), resulting in increased serum concentrations of LDL-cholesterol (LDL-C). Gain-of-function mutations such as S127R and F216L are associated with autosomal dominant hypercholesterolemia, while loss-of-function mutations are linked to low plasma LDL-C levels and a reduced risk of cardiovascular disease.

PCSK9 is autocatalytically cleaved between Q152 and S153 in the endoplasmic reticulum to form the 14 kDa prodomain and 63 kDa mature protein. The prodomain remains closely-associated with the catalytic domain of the mature protein, blocking the substrate binding site. After the ER, PCSK9 either progresses through the extracellular or intracellular pathways. In the extracellular pathway, PCSK9 is secreted from the cell via the Golgi network, and binds to extracellular LDLR. PCSK9: LDLR complexes are internalised by cells via clathrin-mediated endocytosis and trafficked to the endosomes, resulting in degradation of LDLR. In the intracellular pathway, PCSK9 is sorted to lysosomes together with LDLR, leading to degradation of LDLR.

PCSK9 has been shown to bind to LDLR via interaction between the catalytic domain of PCSK9 and the EGF-like repeat homology domain of LDLR. Studies have shown that inactivation of the catalytic domain of PCSK9 does not inhibit LDLR degradation, indicating that secreted PCSK9 acts as a chaperone for LDLR degradation, rather than as a catalytic enzyme.

Plasma LDL-C is mainly cleared through the LDL receptor (LDLR) pathway. The LDLR: LDL-C pathway and the role of PCSK9 in its regulation is described e.g. in Gu and Zhang J Biomed Res. (2015) 29 (5): 356-361, which is hereby incorporated by reference in its entirety.

An inhibitory nucleic acid reducing expression of PCSK9 has been shown to be an effective treatment for reducing serum levels of LDL-C, and thus for the treatment and prevention of hypercholesterolemia and associated diseases (e.g. cardiovascular diseases). See e.g. Raal et al., N Engl J Med (2020) 382 (16): 1520-1530, Ray et al., N Engl J Med. (2020) 382 (16): 1507-1519 and Stoekenbroek et al., Future Cardiol. (2018) 14 (6): 433-442, all of which are hereby incorporated by reference in its entirety. The inhibitory nucleic acid inclisiran is approved in the EU for the treatment of dyslipidemias and hypercholesterolemia.

### Therapeutic and Prophylactic Applications

The inhibitory nucleic acids, nucleic acids, expression vectors, cells and compositions described herein find use in therapeutic and prophylactic methods.

The present disclosure provides an inhibitory nucleic acid, nucleic acid, expression vector, or composition described herein for use in a method of medical treatment or prophylaxis. Also provided is the use of an inhibitory nucleic acid, nucleic acid, expression vector, or composition described herein in the manufacture of a medicament for treating or preventing a disease or condition. Also provided is a method of treating or preventing a disease or condition, comprising administering to a subject a therapeutically or prophylactically effective amount of an inhibitory nucleic acid, nucleic acid, expression vector, or composition described herein.

The terms “disorder”, “disease” and “condition” may be used interchangeably and refer to a pathological issue of a body part, organ or system which may be characterised by an identifiable group of signs or symptoms.

### Subjects

A subject in accordance with the various aspects of the present disclosure may be any animal or human. Therapeutic and prophylactic applications may be in human or animals (veterinary use). The subject to be treated with a therapeutic substance described herein may be a subject in need thereof. The subject is preferably mammalian, more preferably human. The subject may be a non-human mammal, but is more preferably human. The subject may be male or female. The subject may be a patient.

A subject may have been diagnosed with a disease or condition described herein, may be suspected of having such a disease/condition, or may be at risk of developing/contracting such a disease/condition. In embodiments according to the present disclosure, a subject may be selected for treatment according to the methods based on characterisation for certain markers of such disease/condition.

### Kits

In some aspects of the present disclosure a kit of parts is provided. In some embodiments the kit may have at least one container having a predetermined quantity of an inhibitory nucleic acid, nucleic acid, expression vector, cell or composition described herein.

In some embodiments, the kit may comprise materials for producing an inhibitory nucleic acid, nucleic acid, expression vector, cell or composition described herein.

The kit may provide the inhibitory nucleic acid, nucleic acid, expression vector, cell or composition together with instructions for administration to a patient in order to treat a specified disease/condition.

In some embodiments the kit may further comprise at least one container having a predetermined quantity of another therapeutic agent (e.g. as described herein). In such embodiments, the kit may also comprise a second medicament or pharmaceutical composition such that the two medicaments or pharmaceutical compositions may be administered simultaneously or separately such that they provide a combined treatment for the specific disease or condition.

Kits according to the present disclosure may include instructions for use, e.g. in the form of an instruction booklet or leaflet. The instructions may include a protocol for performing any one or more of the methods described herein.

The invention includes the combination of the aspects and preferred features described except where such a combination is clearly impermissible or expressly avoided.

The section headings used herein are for organisational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Where a nucleic acid sequence is disclosed herein, the reverse complement thereof is also expressly contemplated.

Methods described herein may be performed in vitro or in vivo. In some embodiments, methods described herein are performed in vitro. The term “in vitro” is intended to encompass experiments with cells in culture whereas the term “in vivo” is intended to encompass experiments with intact multi-cellular organisms.

### BRIEF DESCRIPTION OF THE FIGURES

Embodiments and experiments illustrating the principles of the invention will now be discussed with reference to the accompanying figures.

FIG. 1. IC50 of (ptGal)₃-siRNA conjugate in transfection-based condition. IC50 curves showing PCSK9 protein knockdown efficacy of (ptGal)₃-siRNA, triGal-siRNA, and Leqvio, respectively. The three conjugates have the same siRNA sequence, which is the sequence of the passenger strand of inclisiran. TriGal-siRNA represents inclisiran synthesized in house, and Leqvio® is clinical grade inclisiran.

FIG. 2. IC50 of (tGal)₃-siRNA conjugate in transfection-based condition. IC50 curves showing PCSK9 protein knockdown efficacy of (tGal)₃-SiRNA and triGal-siRNA respectively. The two conjugates have the same siRNA sequence, which is the sequence of the passenger strand of inclisiran. TriGal-siRNA represents inclisiran synthesized in house.

FIG. 3. IC50 of (gGal)₃-siRNA conjugate in transfection-based condition. IC50 curves showing PCSK9 protein knockdown efficacy of (gGal)₃-SiRNA and triGal-siRNA respectively. The two conjugates have the same siRNA sequence, which is the sequence of the passenger strand of inclisiran. TriGal-siRNA represents inclisiran synthesized in house.

FIG. 4. In vivo PCSK9 knockdown using (ptGal)₃-siRNA conjugate. A) Graph showing the in vivo efficacy on PCSK9 protein reduction and duration for the indicated siRNAs in hPCSK9-KI mice. siRNAs were administrated through subcutaneous injection at a single dose of 2 mg/kg. The hPCSK9 protein serum levels were determined by ELISA. B) Graph showing the mean level of hPCSK9 at various time points (day 2 pre-injection, day 1 pre-injection & day 5, day 11, day 15, day 22, day 29, day 43 and day 49 post-injection with the siRNA).

FIG. 5. In vivo PCSK9 knockdown using (tGal)₃-siRNA conjugate. A) Graph showing the in vivo efficacy on PCSK9 protein reduction and duration for the indicated siRNAs in hPCSK9-KI mice. siRNAs were administrated through subcutaneous injection at a single dose of 2 mg/kg. The hPCSK9 protein serum levels were determined by ELISA. B) Graph showing the mean level of hPCSK9 at various time points (day 13 pre-injection, day 8 pre-injection & day 4, day 11, day 18, day 25 and day 32 post-injection with the siRNA).

FIG. 6. In vivo PCSK9 knockdown using (gGal)₃-siRNA conjugate. A) Graph showing the in vivo efficacy on PCSK9 protein reduction and duration for the indicated siRNAs in hPCSK9-KI mice. siRNAs were administrated through subcutaneous injection at a single dose of 2 mg/kg. The hPCSK9 protein serum levels were determined by ELISA. B) Graph showing the mean level of hPCSK9 at various time points (day 9 pre-injection, day 3 pre-injection & day 4, day 11, day 15, day 22, day 29, day 43 and day 49 post-injection with the siRNA).

FIGS. 7A to 7C. GalNAc-oligonucleotide conjugates prepared according to the invention.

FIGS. 8A to 8C. GalNAc-siRNA conjugates prepared according to the invention.

### EXAMPLES

In the following Examples, the inventors describe the synthesis of GalNAc-linkers, GalNAc monomer precursors, GalNAc amidite monomer, GalNAc solid support and GalNAc siRNA conjugates.

### GalNAc-Linkers GalNAc-linkers are intermediate compounds for use in the synthesis of the GalNAc monomer precursors described herein.

### GalNAc-Linker 1

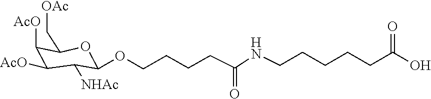 [SMILES: CC(=O)NC1C(OCCCCC(=O)NCCCCCC(=O)O)OC(COC(C)=O)C(OC(C)=O)C1OC(C)=O]

GalNAc-linker acid 1 may be synthesised as shown in Scheme 1. Peracylated GalNAc is reacted with TMSOTf to remove the C-1 acetate group and the resulting oxonium ion is trapped by the intramolecular cyclisation of the-NHAc group. The alcohol is attached at the C-1 position using TMSOTf and 4 Å molecular sieves. The terminal alkene is oxidatively cleaved and oxidised to the carboxylic acid using sodium periodate and ruthenium (III) chloride. The resulting carboxylic acid is coupled to a terminal amine to form GalNAc-linker acid 1.

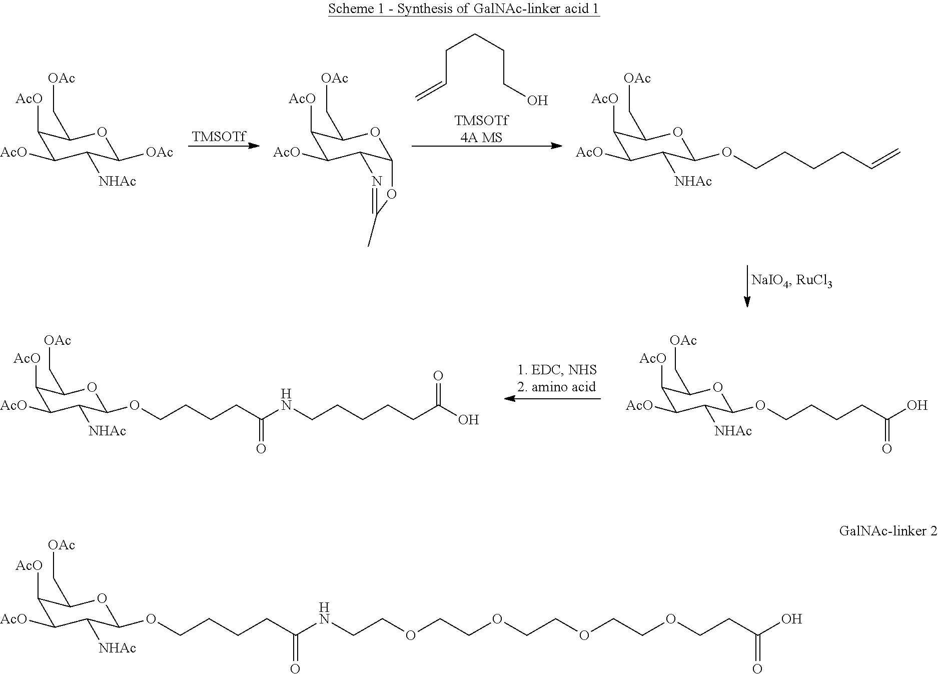

The inventors postulate that GalNAc-linker acid 2 may be synthesised as shown in Scheme 2. Peracylated GalNAc is reacted with TMSOTf to remove the C-1 acetate group and the resulting oxonium ion is trapped by the intramolecular cyclisation of the-NHAc group. The alcohol is attached at the C-1 position using TMSOTf and 4 Å molecular sieves. The terminal alkene is oxidatively cleaved and oxidised to the carboxylic acid using sodium periodate and ruthenium (III) chloride. The resulting carboxylic acid is coupled to a terminal amine to form GalNAc-linker acid 2.

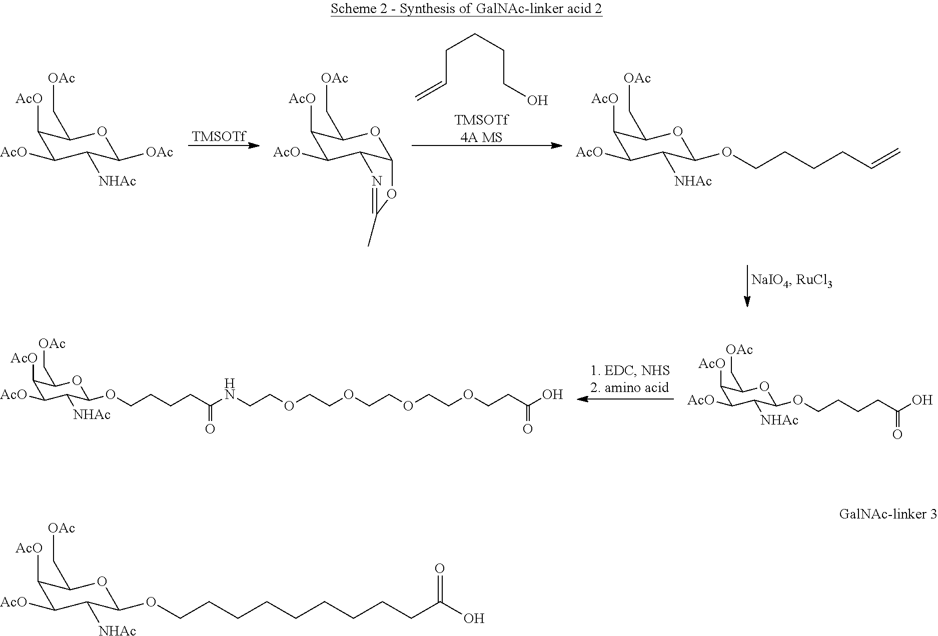

The inventors postulate that GalNAc-linker acid 3 may be synthesised as shown in Scheme 3. Peracylated GalNAc is reacted with TMSOTf to remove the C-1 acetate group and the resulting oxonium ion is trapped by the intramolecular cyclisation of the-NHAc group. The alcohol is attached at the C-1 position using TMSOTf and 4 Å molecular sieves. The terminal alkene is oxidatively cleaved and oxidised to the carboxylic acid using sodium periodate and ruthenium (III) chloride to form GalNAc-linker acid 3.

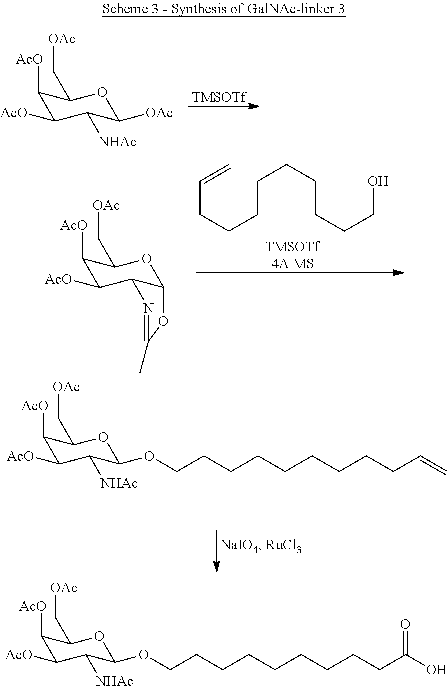

Other GalNAc linker precursors disclosed herein include GalNAc-linkers 4 to 10. These are shown below and may be synthesised in line with methods described in Examples 2 to 8:

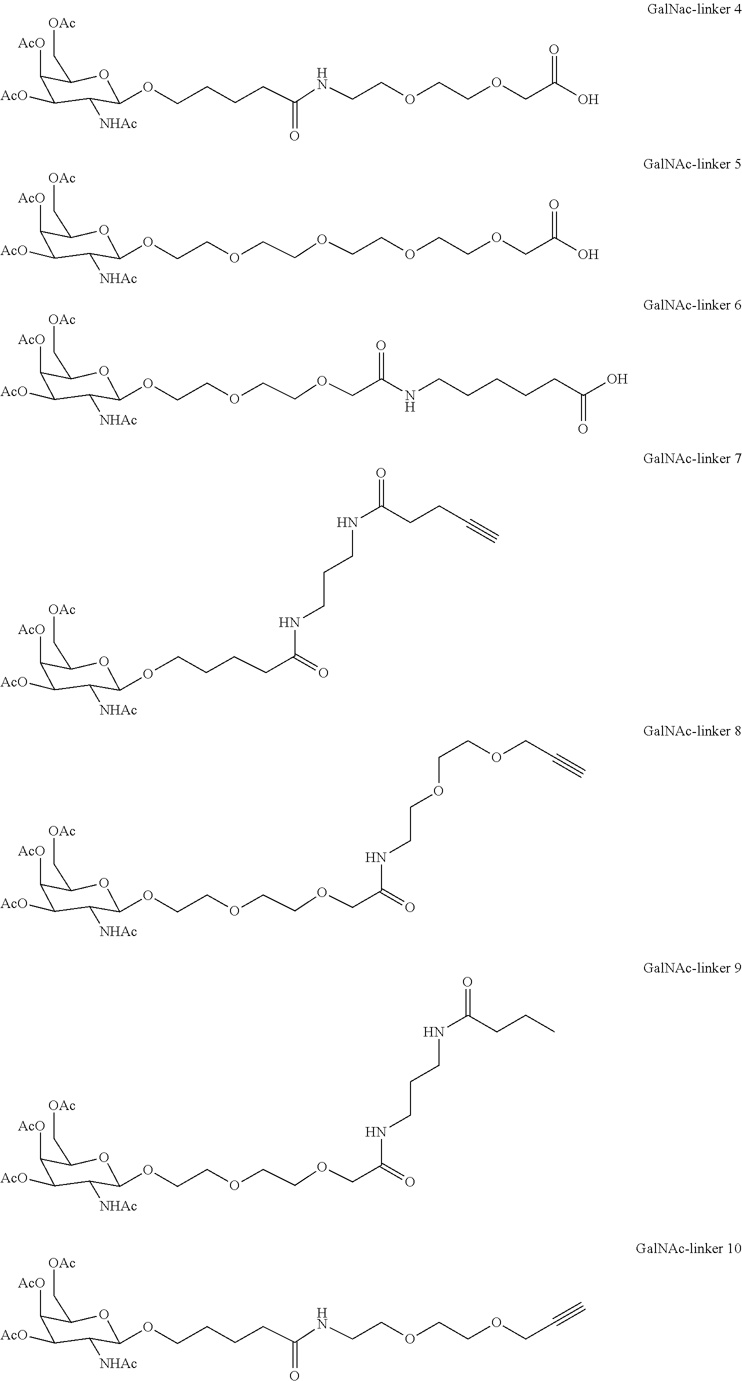

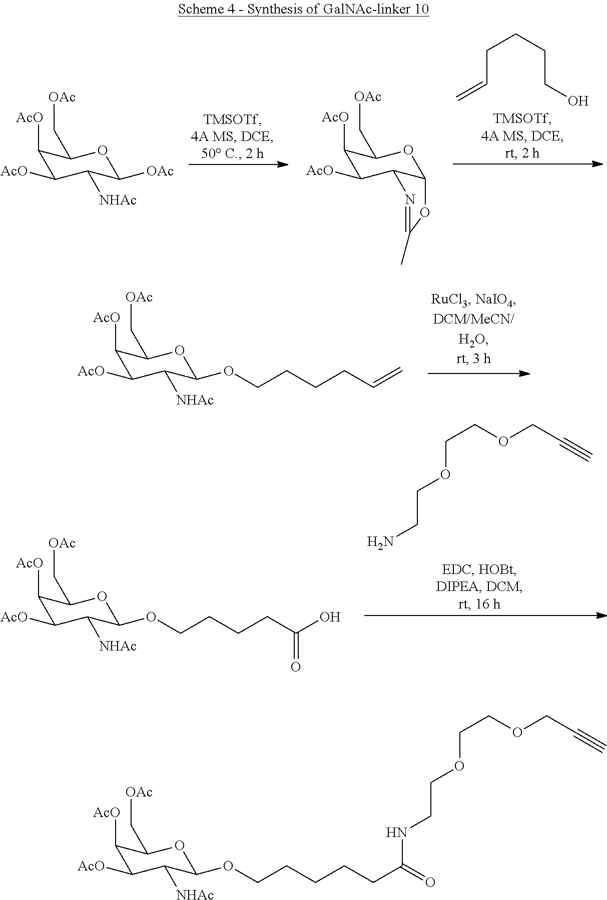

### GalNAc Monomer Precursors GalNAc monomer precursors are intermediate compounds for use in the synthesis of the GalNAc amidite monomers and GalNAc solid supports described herein.

### GalNAc Monomer Precursor 1 (from GalNAc Linker 1)

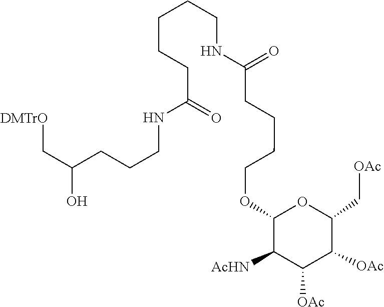

### GalNAc Monomer Precursor 2 (from GalNAc Linker 4)

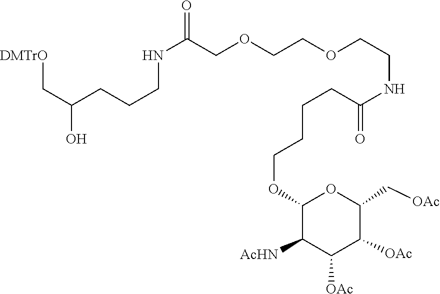

### GalNAc Monomer Precursor 3 (from GalNAc Linker 5)

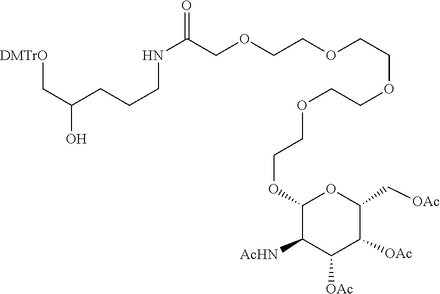

### GalNAc Monomer Precursor 4 (from GalNAc Linker 6)

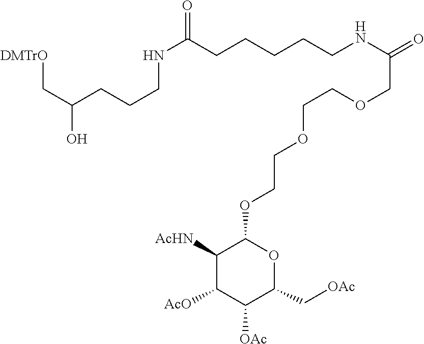

### GalNAc Monomer Precursor 5 (from GalNAc Linker 10)

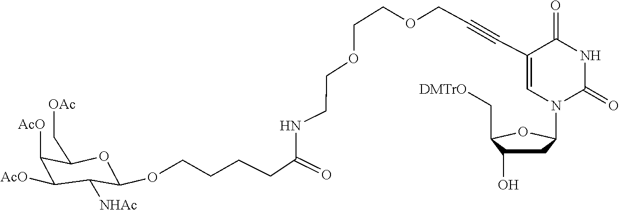

### GalNAc Monomer Precursor 6 (from GalNAc Linker 10)

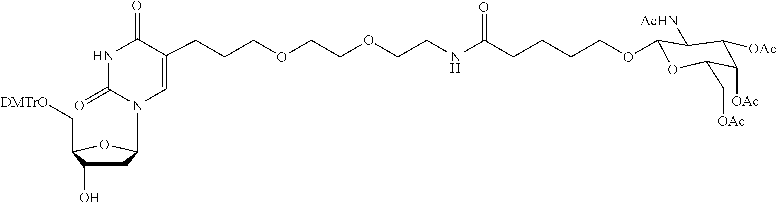

### GalNAc Monomer Precursor 7 (from GalNAc Linker 7)

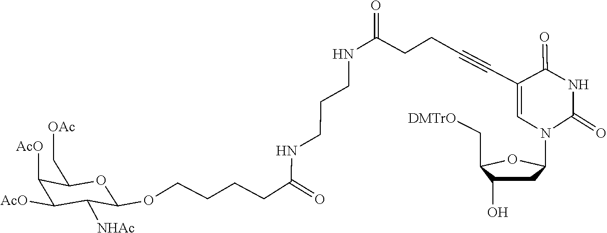

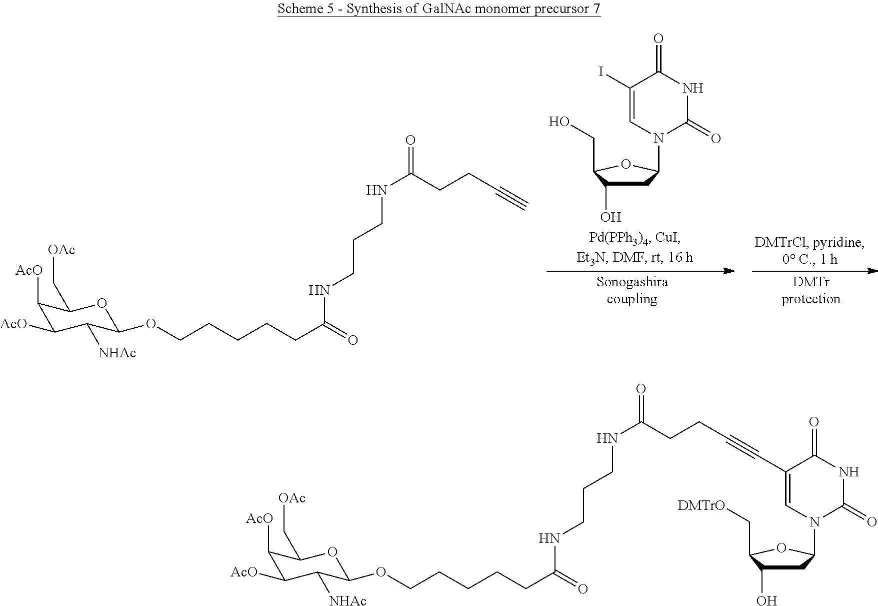

### GalNAc Monomer Precursor 8 (from GalNAc Linker 8)

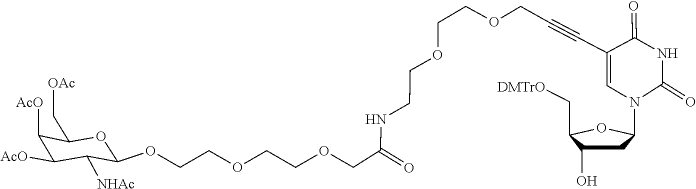

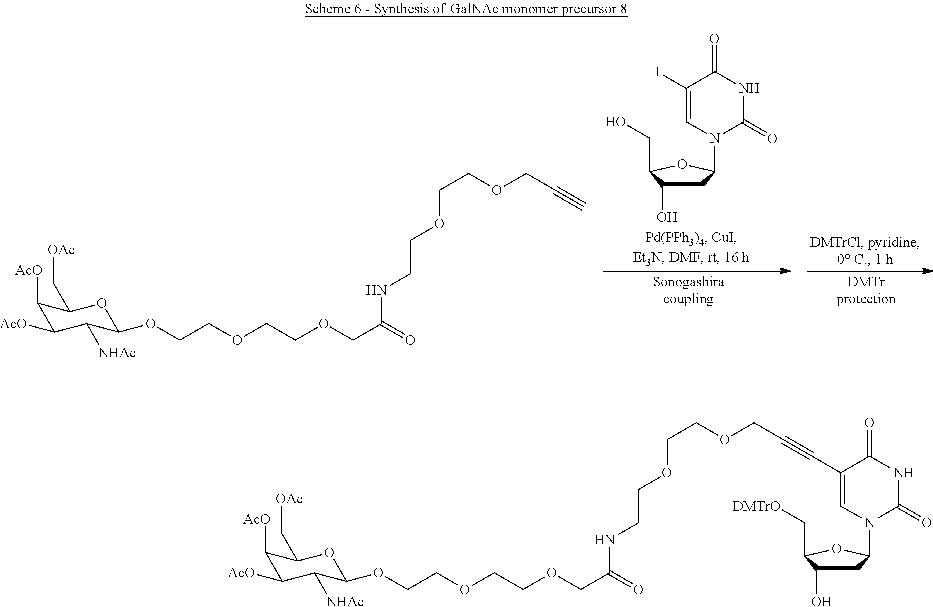

### GalNAc Monomer Precursor 9 (from GalNAc Linker 9)

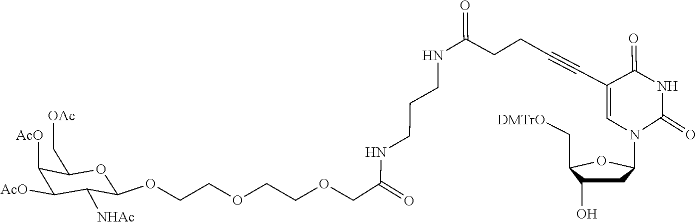

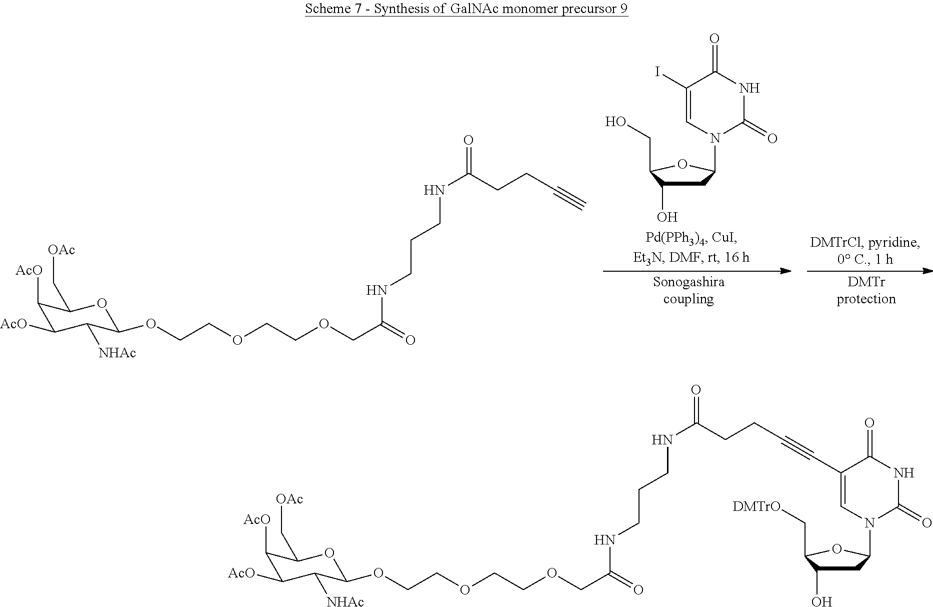

### GalNAc Amidite Monomers General synthetic routes for GalNAc amidite monomers described herein are provided below. Other routes and reagents may be apparent to the skilled person based on the suggested routes below. For example, it will be appreciated that phosphitylation protocols such as the one described in Scheme 8 below are well-known in the art. GalNAc monomer precursors described above can be easily converted to respective GalNAc amidite monomers via phosphitylation protocols as shown in the examples.

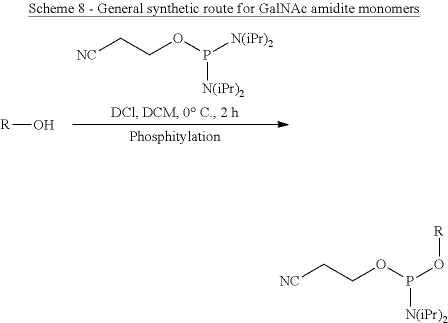

### GalNAc Amidite Monomer 1

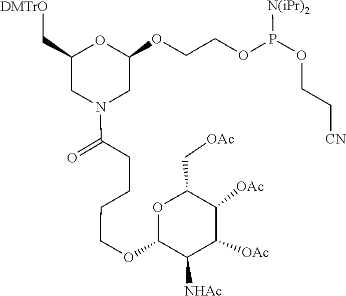

The inventors postulate that GalNAc monomer 1 may be synthesised as shown in Scheme 9. Peracylated ribofuranose is reacted with boron trifluoride diethyl etherate and an alcohol to attach the alcohol at the C-1 position. Deprotection of all the remaining acetyl groups was performed with K₂CO₃ and methanol. The primary hydroxyl group is protected with DMTrCI and pyridine. The remaining 1,2-diol is oxidatively cleaved with sodium periodate then the reductive amination with ammonia and sodium triacetoxyborohydride is performed to synthesise the morpholino moiety. The GalNAc-linker acid is coupled to the nitrogen of the morpholino moiety, and the benzyl group is removed using hydrogenation. Finally, the free hydroxyl group is coupled to the phosphorodiamidite with DCI to form GalNAc monomer 1.

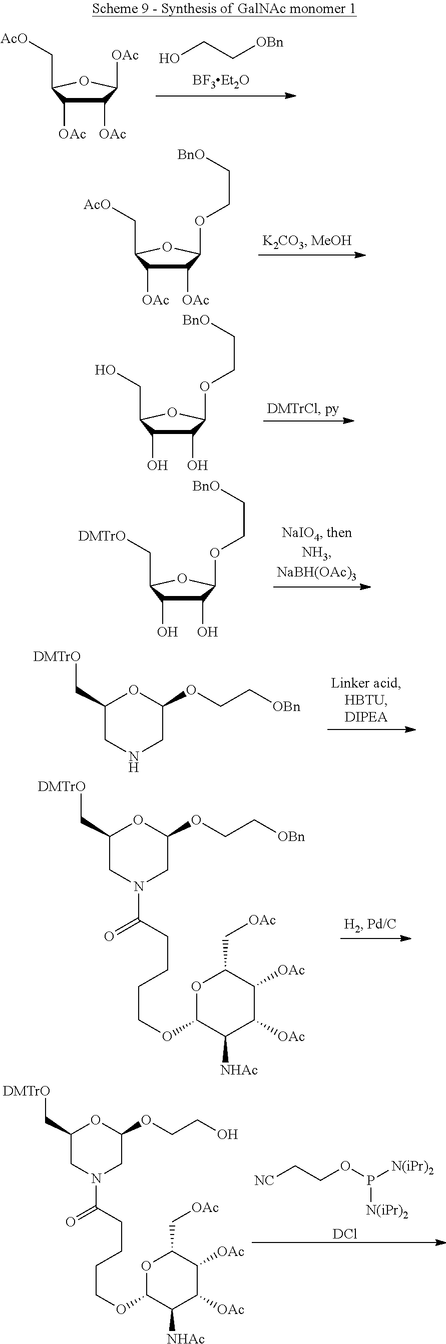

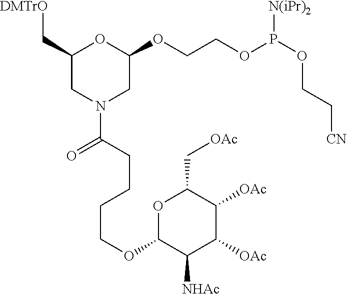

### GalNAc Amidite Monomer 2

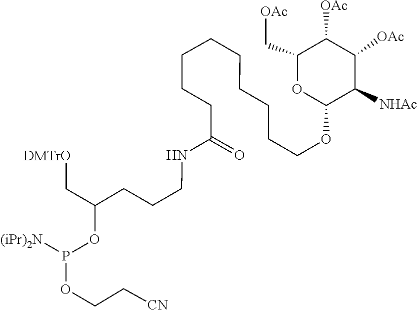

The inventors postulate that GalNAc amidite monomer 2 may be synthesised as shown in Scheme 10.

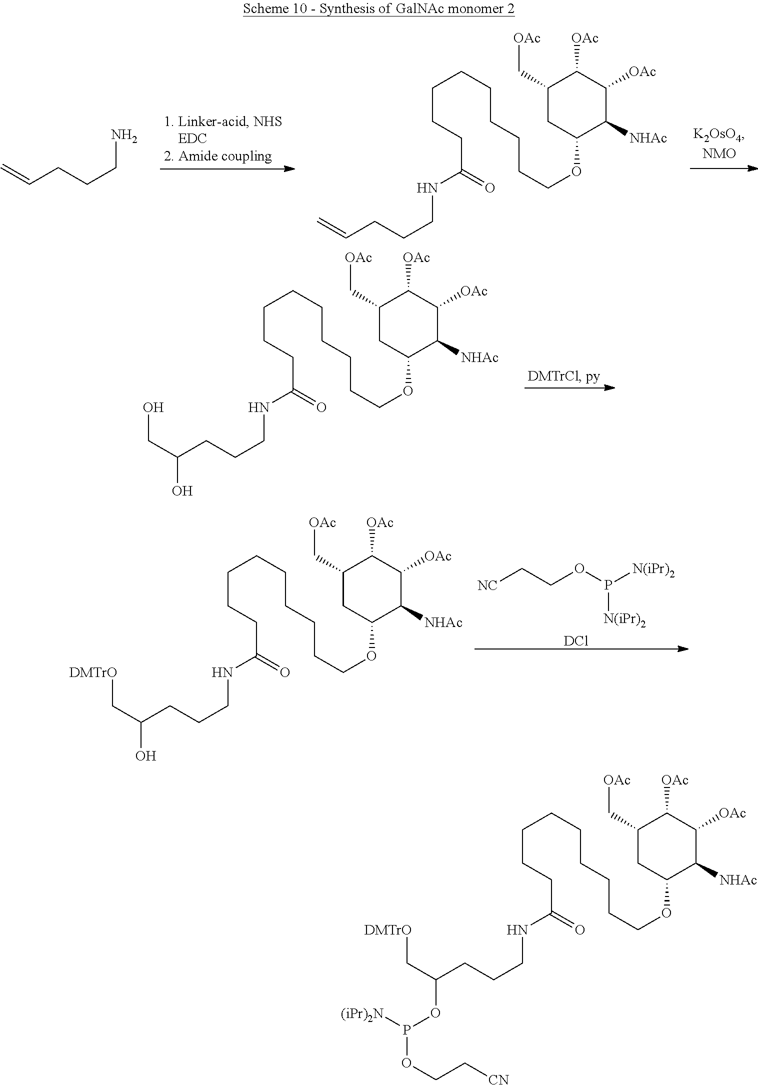

### GalNAc Amidite Monomer 3 (ptGal Phosphoremidite)

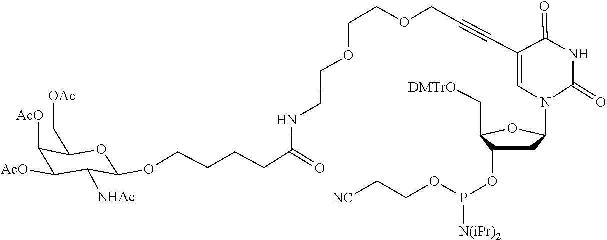

GalNAc Amidite Monomer 3 (ptGal phosphoramidite) may be synthesised as shown in Scheme 11.

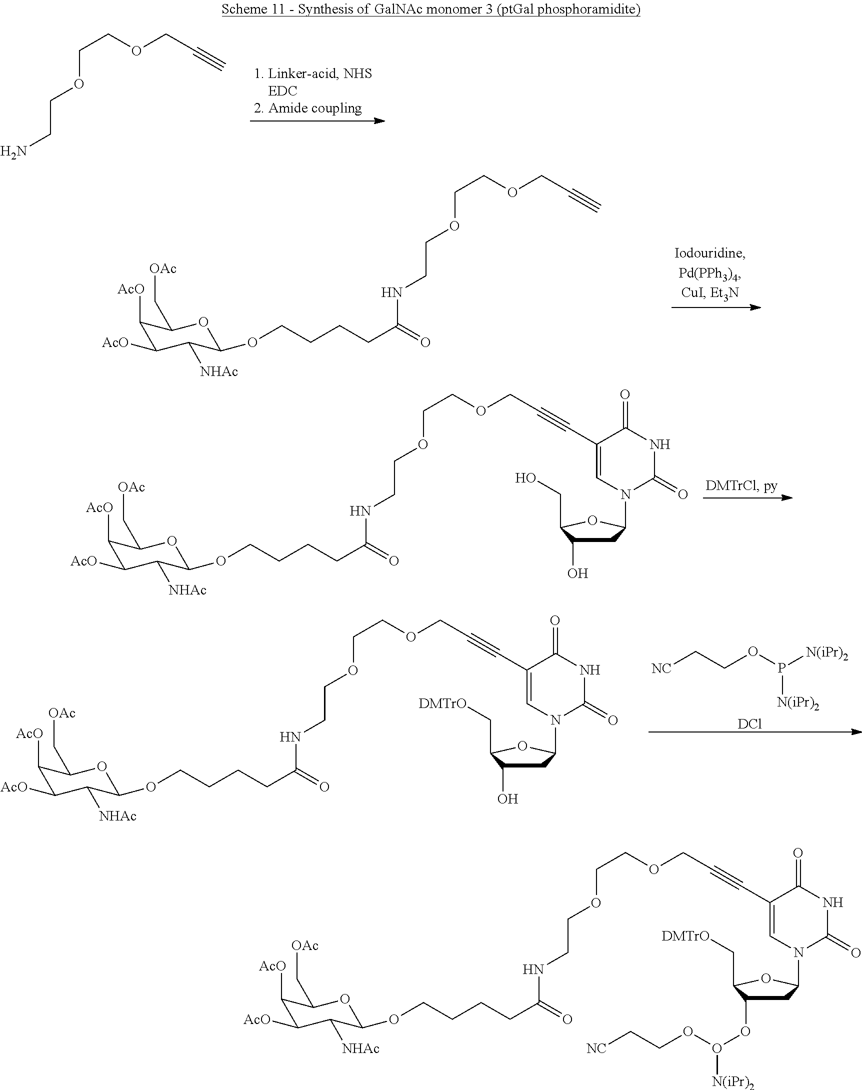

GalNAc Amidite Monomer 5 (tGal Phosphoramidite)

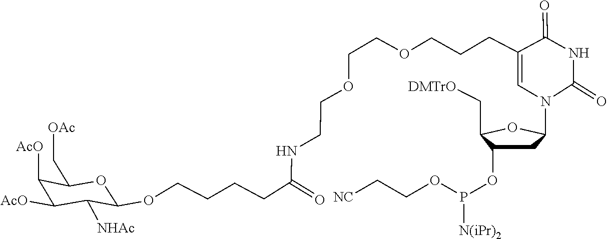

GalNAc Amidite Monomer 6 (gGal Phosphoramidite)

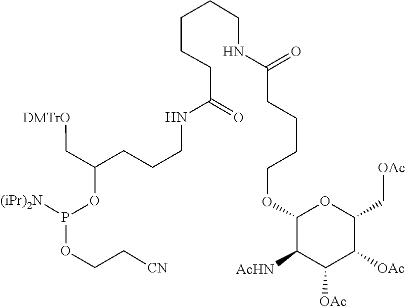

### GalNAc Solid Support

General synthetic routes for GalNAc monomers described herein are provided below. Other routes and reagents may be apparent to the skilled person based on the suggested routes below. The GalNAc monomer precursor reacts with succinic anhydride and the resulted succinic acid is coupled with amino group functionalized solid support (eg. Controlled pore glass (CPG) or polystyrene). After capping with acetic anhydride and washing, GalNAc solid support can be achieved and its loading amount can be evaluated via UV evaluation of removed DMTr anion. GalNAc monomer precursors described above can be easily converted to respective GalNAc solid supports via loading protocols as shown in the examples.

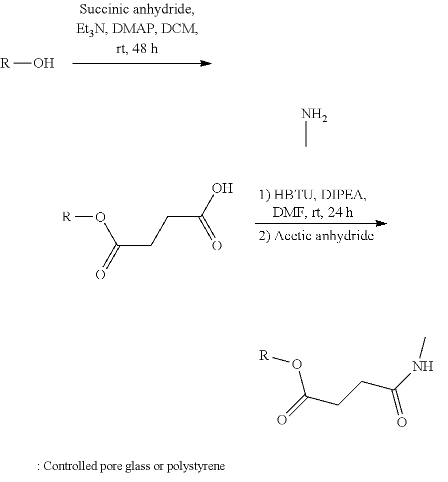

### GalNAc Solid Support 1 (qGal CPG)

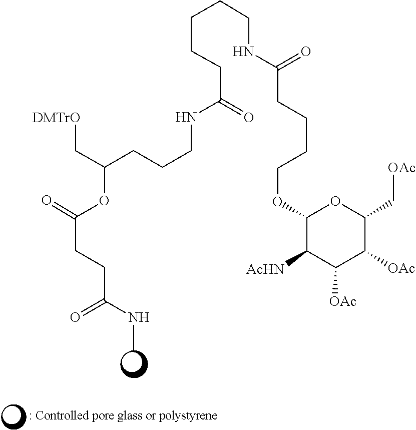

### GalNAc Solid Support 2 (ptGal CPG)

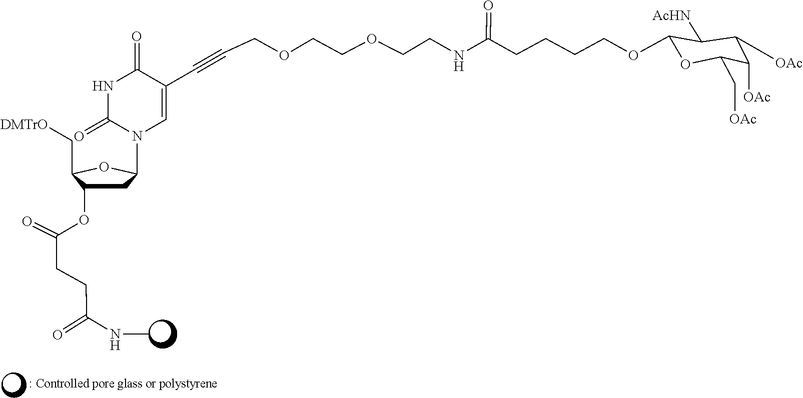

### GalNAc Solid Support 3 (tGal CPG)

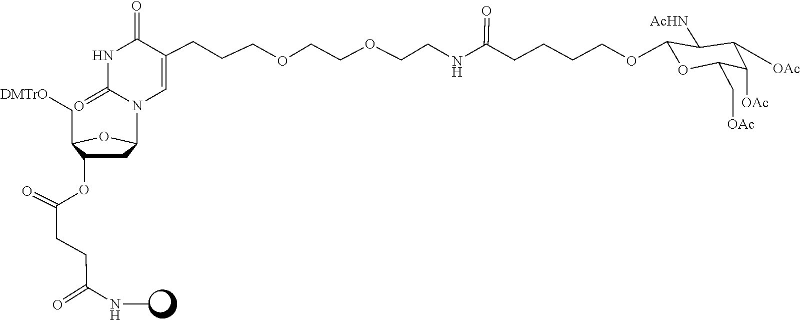

### GalNAc Conjugated Oligonucleotide and siRNA Synthesis

GalNAc conjugated oligonucleotide can be synthesized via oligonucleotide synthesizer using GalNAc amidite monomer and/or GalNAc solid support described above together with commercially available amidite monomers and/or solid supported nucleotides. For example, the three GalNAc moieties may be attached using three amidite monomers, or a combination of solid supported monomer(s) and amidite monomer(s).

After cleavage and deprotection, crude GalNAc conjugated oligonucleotide can be obtained. Depending on the downstream application, different purification techniques can be used to purify the GalNAc conjugated oligonucleotide. By annealing to the complimentary sequence, a GalNAc conjugated duplex (eg. GalNAc siRNA) can be obtained.

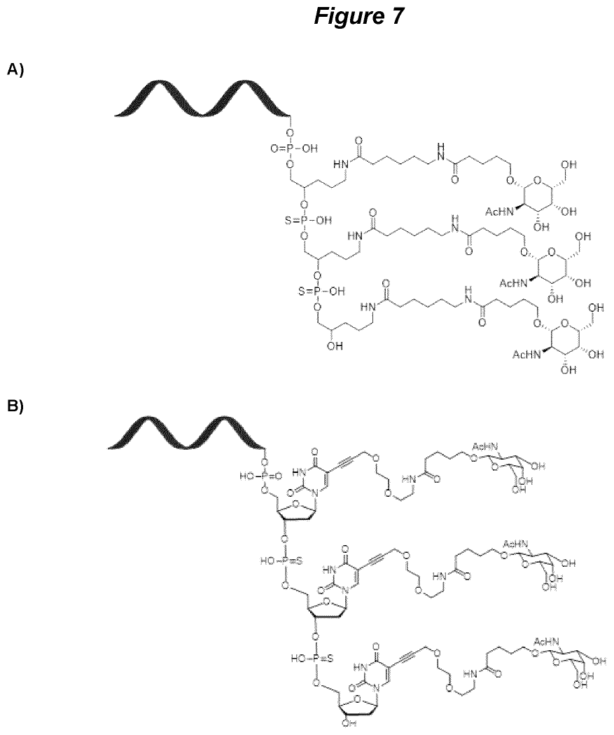

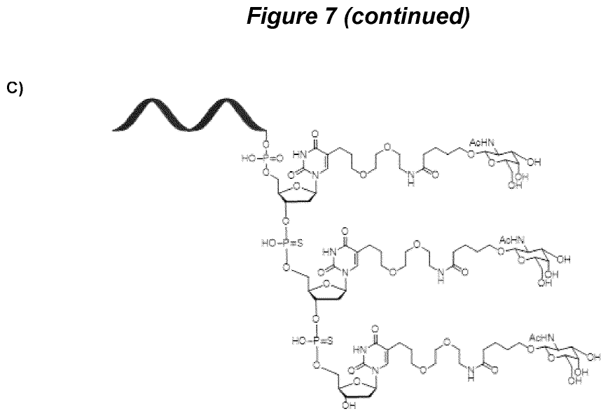

GalNAc conjugated oligonucleotide 1 ((gGal)₃-oligo) is shown in FIG. 7A.

GalNAc conjugated oligonucleotide 2 ((ptGal)₃-oligo) is shown in FIG. 7B.

GalNAc conjugated oligonucleotide 3 ((tGal)₃-oligo) is shown in FIG. 7C.

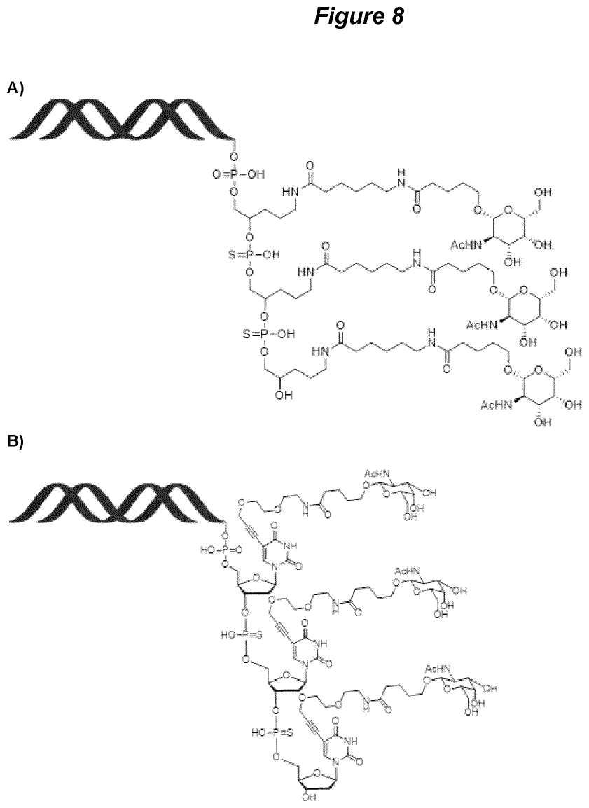

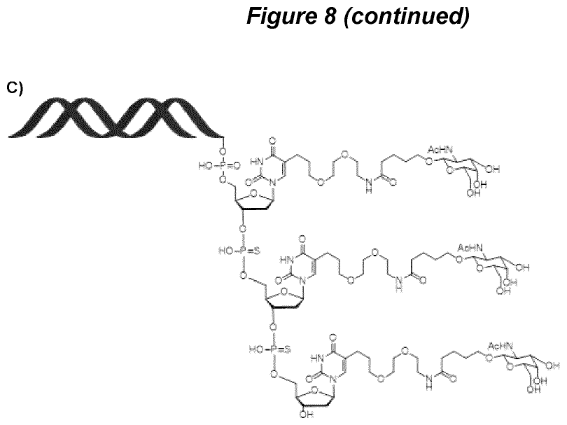

GalNAc conjugated siRNA 1 ((gGal)₃-siRNA) is shown in FIG. 8A.

GalNAc conjugated siRNA 2 ((ptGal)₃-siRNA) is shown in FIG. 8B.

GalNAc conjugated siRNA 3 ((tGal)₃-SIRNA) is shown in FIG. 8C.

### Example 1—Synthesis and Characterisation of GalNAc Linker 1

GalNAc linker 1 was synthesized according to Scheme 12. The stepwise reaction procedures are described in the following paragraphs.

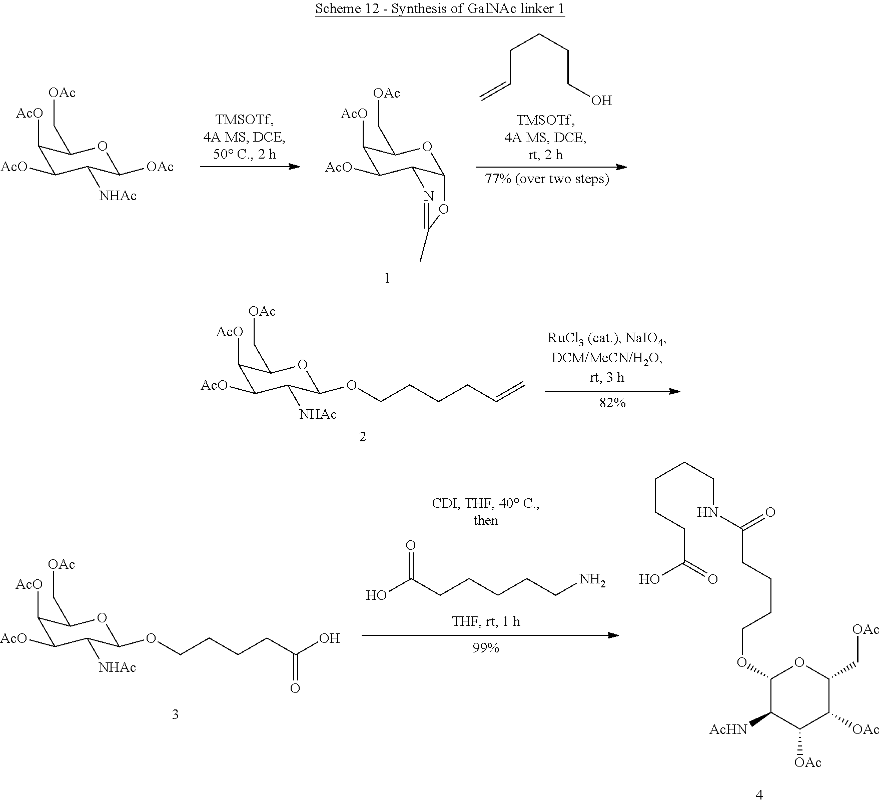

Galactosamine pentaacetate (30 g, 77.0 mmol) was dissolved in 1,2-dichloroethane (200 mL) and stirred over 4 Å molecular sieves for 30 min. Trimethylsilyl trifluoromethanesulfonate (15.3 mL, 18.8 mmol) was added at room temperature and stirred at 50° C. for 2 h. The reaction was cooled to 0° C. and slowly quenched with triethylamine (43.0 mL, 308 mmol), then stirred at 0° C. for 15 min and at room temperature for 15 min. The reaction mixture was filtered, washing the filter with dichloromethane (3×150 mL). The filtrate was washed with saturated aqueous sodium bicarbonate (3×150 mL), then dried over sodium sulfate, filtered and concentrated under reduced pressure to afford compound 1 (25.3 g, 76.9 mmol, 100%) which was used without further purification.

Compound 1 (25.3 g, 76.9 mmol) and 5-hexen-1-ol (12.0 mL, 100 mmol) were dissolved in 1,2-dichloromethane (200 mL), then stirred over 4 Å molecular sieves for 30 min. Trimethylsilyl trifluoromethanesulfonate (7.0 mL, 38.5 mmol) was added at room temperature and stirred at room temperature for 1.5 h. The reaction was cooled to 0° C., diluted with dichloromethane (150 mL) and quenched with saturated aqueous sodium bicarbonate (100 mL). The layers were separated and the aqueous phase was extracted with dichloromethane (2×150 mL). The combined organic extracts were dried over sodium sulfate, filtered and concentrated under reduced pressure. The crude product was purified by column chromatography (0%->20%->50%->70% ethyl acetate/hexane) to give compound 2 (25.6 g, 59.6 mmol, 77%) as a light yellow solid.

¹H NMR (400 MHZ, DMSO) δ 7.82 (d, J=9.2 Hz, 1H), 5.79 (ddt, J=16.9, 10.2, 6.6 Hz, 1H), 5.21 (d, J=3.4 Hz, 1H), 5.05-4.92 (m, 3H), 4.48 (d, J=8.4 Hz, 1H), 4.09-3.98 (m, 3H), 3.87 (dt, J=11.3, 8.9 Hz, 1H), 3.71 (dt, J=9.9, 6.0 Hz, 1H), 3.42 (dt, J=10.0, 6.4 Hz, 1H), 2.10 (s, 3H), 2.05-1.97 (m, 2H), 2.00 (s, 3H), 1.89 (s, 3H), 1.76 (s, 3H), 1.53-1.43 (m, 2H), 1.41-1.30 (m, 2H).

Compound 2 (32.8 g, 76.4 mmol) was dissolved in a mixture of dichloromethane (100 mL), acetonitrile (100 mL) and water (160 mL). Sodium metaperiodate (65.3 g, 306 mmol) and ruthenium (III) chloride (316 mg, 1.53 mmol) were added to the solution. The reaction was stirred for at room temperature for 1.5 h, using a water bath to control any exotherm. A further portion of sodium metaperiodate (16.3 g, 76.4 mmol) was added and the reaction was stirred at room temperature for 1 h. The reaction was filtered, washing the filter with dichloromethane (3×200 mL) and quenching the filtrate with saturated aqueous sodium bicarbonate (160 mL), checking that the aqueous phase reaches pH 8-9. The layers were separated and the aqueous phase was washed with DCM (2×100 mL), then acidified with aqueous 4 N hydrochloric acid (˜50 mL) to pH 3-4 and extracted with DCM (8×200 mL). The combined organic extracts were dried over Na₂SO₄, filtered, and evaporated to give compound 3 (28.2 g, 63.0 mmol, 82%) as a white foam that was used without further purification.

¹H NMR (400 MHZ, DMSO) δ 12.00 (s, 1H), 7.82 (d, J=9.2 Hz, 1H), 5.21 (d, J=3.4 Hz, 1H), 4.96 (dd, J =11.2, 3.4 Hz, 1H), 4.49 (d, J=8.4 Hz, 1H), 4.06-3.98 (m, 3H), 3.88 (dt, J=11.3, 8.9 Hz, 1H), 3.76-3.66 (m, 1H), 3.44-3.39 (m, 1H), 2.23-2.14 (m, 2H), 2.10 (s, 3H), 2.00 (s, 3H), 1.89 (s, 3H), 1.77 (s, 3H), 1.56-1.43 (m, 4H).

Compound 3 (10.2 g, 22.8 mmol) was dissolved in tetrahydrofuran (150 mL) and 1,1′-carbonyldiimidazole (4.81 g, 29.6 mmol) was added at room temperature. The reaction was stirred at 40° C. for 1.5 h, then treated with a solution of 6-aminohexanoic acid (4.49 g, 34.2 mmol) dissolved in 1 M aqueous sodium carbonate (34.2 mL, 34.2 mmol). The reaction was stirred at 40° C. for 1.5 h, then cooled to 0° C., diluted with ethyl acetate (150 mL) and carefully quenched with 4 M HCl (35 mL). The layers were separated and the aqueous phase was extracted with ethyl acetate (4×100 mL). The combined organic extracts were dried with sodium sulfate, filtered and concentrated under reduced pressure to give Compound 4 (12.7 g, 22.6 mmol, 99%) as a white foam which was used without further purification.

### Example 2—Synthesis and Characterisation of GalNAc Linker 4

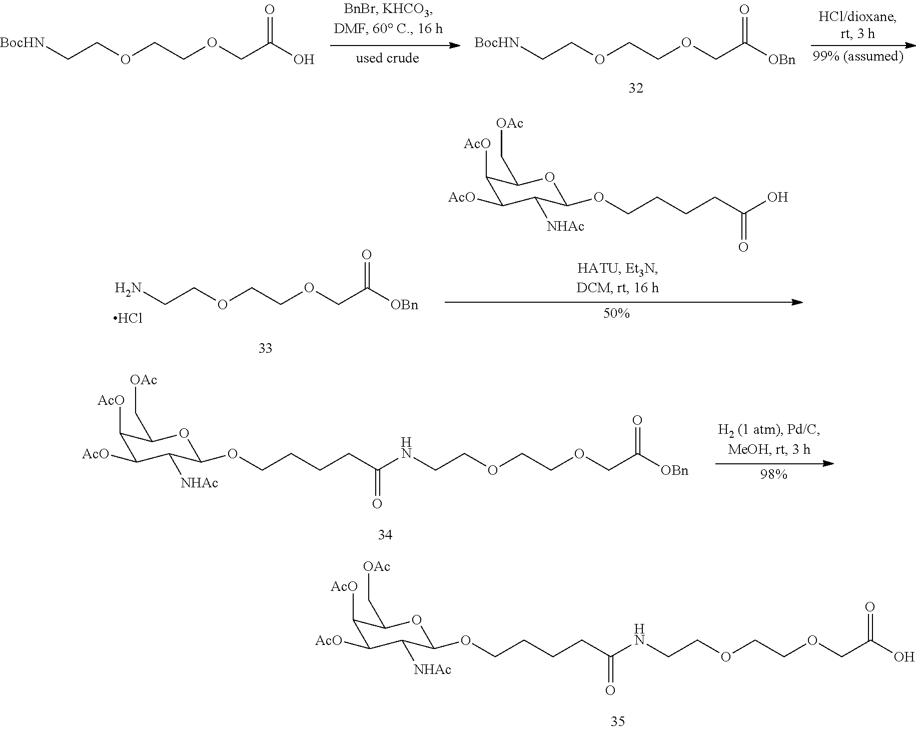

Boc acid (16.6 g, 63.0 mmol), potassium hydrogen carbonate (22.6 g, 164 mmol) and acetone (242 mL) were combined to give a suspension. Benzyl bromide (9.75 mL, 82.0 mmol) was added and the flask was stirred at 60° C. for 16 h under an atmosphere of argon. The reaction was filtered through a pad of Celite, washing with dichloromethane (2×50 mL) and concentrated. The crude product was purified by column chromatography (0%->50% ethyl acetate/hexanes) to afford compound 32 which was used without further characterisation.

Compound 32 was treated with hydrochloric acid (4.0 M in dioxane, 63 mL, 252 mmol). The reaction mixture was stirred at room temperature for 16 h, then concentrated to a residue. The residue was washed with methyl tert-butyl ether (2×50 mL) to afford compound 33 as a hygroscopic solid (17.5 g, 60.4 mmol, 96%) which was used without further purification.

Compound 33 (18.2 g, 40.8 mmol) was dissolved in dichloromethane (154 mL), then HATU (15.5 g, 40.8 mmol), triethylamine (17.0 mL, 122.4 mmol) and GalNAc acid (11.8 g, 40.8 mmol) were added sequentially. The reaction was stirred at room temperature overnight, then poured into aqueous citric acid (10% w/v, 100 mL). The layers were separated and the aqueous phase was extracted with dichloromethane (2×50 mL). The combined organic extracts were dried with sodium sulfate, filtered and concentrated under reduced pressure. The crude product was purified by column chromatography (0%->4% methanol/ethyl acetate) to afford Compound 34 as an white solid (24.9 g, 36.5 mmol, 89%).

Compound 34 (11.8 g, 17.3 mmol) was dissolved in methanol (64 mL) and palladium on carbon (10 wt %, 1.84 g, 1.73 mmol) was added. The reaction was stirred under an atmosphere of hydrogen (1 atm) for 3 h. The mixture was filtered through Celite, washing with methanol (2×50 mL), and dried under high vacuum to afford Compound 35 as a thick yellow oil (9.43 g, 15.9 mmol, 92%).

### Example 3—Synthesis and Characterisation of GalNAc Linker 5

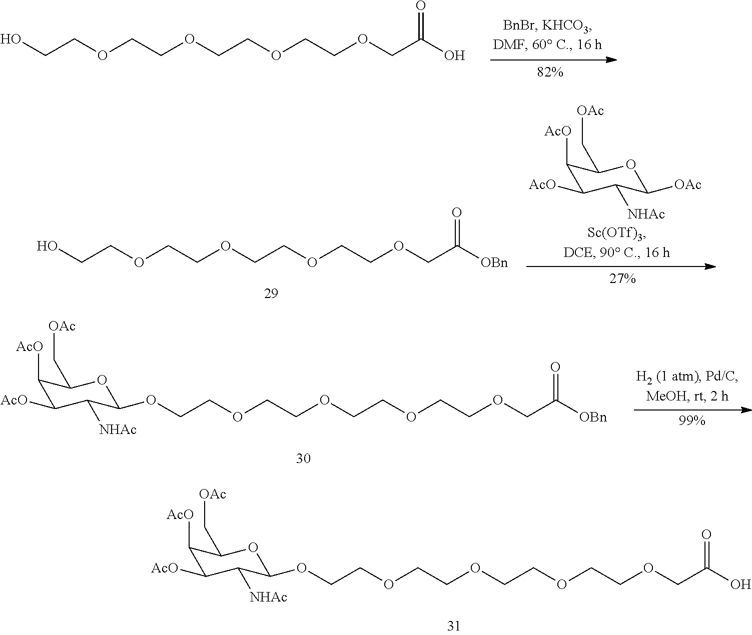 [SMILES: O=C(O)COCCOCCOCCOCCO ; O=C(COCCOCCOCCOCCO)OCc1ccccc1 ; CC(=O)NC1C(OC(C)=O)OC(COC(C)=O)C(OC(C)=O)C1OC(C)=O ; CC(=O)NC1C(OCCOCCOCCOCCOCC(=O)OCc2ccccc2)OC(COC(C)=O)C(OC(C)=O)C1OC(C)=O ; CC(=O)NC1C(OCCOCCOCCOCCOCC(=O)O)OC(COC(C)=O)C(OC(C)=O)C1OC(C)=O]

PEG acid (20.0 g, 79.3 mmol) and potassium hydrogen carbonate (9.52 g, 95.1 mmol) were dissolved in DMF (159 mL) at room temperature. Benzyl bromide (11.3 mL, 95.1 mmol) was added and the reaction was stirred at room temperature for 16 h. The volatiles were removed in vacuo and the residue was taken up in dichloromethane (150 mL) and filtered to remove any insoluble material. The crude product was purified by column chromatography (0%->10% methanol/dichloromethane) to afford Compound 29 (22.4 g, 65.4 mmol, 82%) as a thick yellow oil.

Compound 29 (19.8 g, 57.8 mmol) and galactosamine pentaacetate (17.3 g, 44.4 mmol) were dissolved in 1,2-dichloroethane (170 mL). Scandium (III) triflate (1.53 g, 3.11 mmol) was added and the reaction was stirred at 90°° C. for 16 h. The reaction was cooled to room temperature and poured into aqueous saturated sodium bicarbonate (100 mL) with vigorous stirring. The layers were separated and the aqueous phase was extracted with dichloromethane (3×100 mL). The combined organic extracts were dried with sodium sulfate, filtered and concentrated under reduced pressure. The crude product was purified by column chromatography (0%->10% methanol/dichloromethane) and further purified by reverse phase column chromatography (10%->50% acetonitrile/water) to afford Compound 30 (8.08 g, 12.0 mol, 27%) as a white foam.

Compound 30 (8.08 g, 12.0 mmol) was dissolved in methanol (60 mL) and palladium on carbon (10 wt %, 1.92 g, 1.80 mmol) was added. The reaction was stirred under an atmosphere of hydrogen (1 atm) for 2 h. The mixture was filtered through Celite and concentrated to a semi solid. The residue was purified by reverse phase column chromatography (10%->50% acetonitrile/water) to afford Compound 31 (6.95 g, 11.9 mol, 99%) as a white foam.

¹H NMR (300 MHZ, dmso) δ 7.77 (d, J=9.2 Hz, 1H), 5.19 (d, J=3.5 Hz, 1H), 4.95 (dd, J=11.2, 3.4 Hz, 1H), 4.54 (d, J=8.5 Hz, 1H), 4.00 (d, J=6.9 Hz, 5H), 3.94-3.70 (m, 2H), 3.64-3.44 (m, 15H), 2.09 (s, 3H), 1.98 (s, 3H), 1.87 (s, 3H), 1.76 (s, 3H); carboxylic acid proton not observed.

### Example 4—Synthesis and Characterisation of GalNAc Linker 6

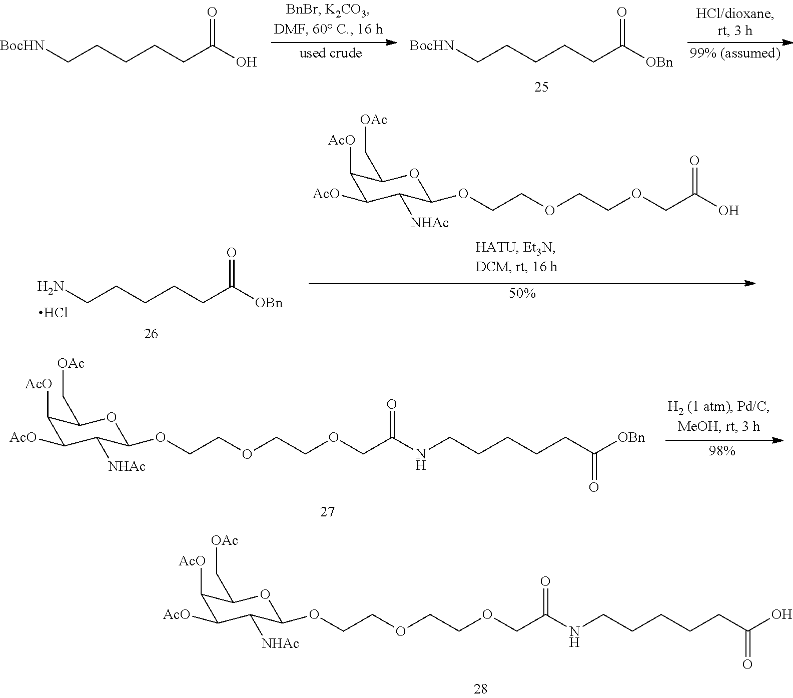

Boc Acid (25.0 g, 108 mmol), potassium carbonate (8.47g, 61.3 mmol) and DMF (400 mL) were combined to give a suspension. Benzyl bromide (2.90 g, 108 mmol) was added and the flask was stirred at 60° C. for 16 h under an atmosphere of argon. The reaction was filtered and concentrated, then the residue was taken up in ethyl acetate (500 mL). The organic phase was sequentially washed with 5% citric acid solution (2×250 mL), water (250 mL) and brine (250 mL), then dried over sodium sulfate, filtered and concentrated to a light yellow oil (30.81 g, 88.7%) which was used without further purification.

The light yellow oil from above was treated with hydrochloric acid (4.0 M in dioxane, 100 mL, 400 mmol). The reaction mixture was stirred at room temperature for 3 h, then concentrated to a residue. The residue was co-evaporated with which was mixed with water (100 mL) and concentrated to give compound 25 as a hygroscopic solid (26.4 g, 107%) which was used without further purification.

Compound 25 (20.0 g, 40.5 mmol) was dissolved in dichloromethane (300 mL), then HATU (16.9 g, 44.4 mmol), triethylamine (17.0 mL, 122.0 mmol) and GalNAc acid (10.4 g, 40.4 mmol) were added sequentially. The reaction was stirred at room temperature overnight, then concentrated to a residue. The crude product was purified by column chromatography (0%->10% methanol/dichloromethane) to obtain a white solid (22.3 g). This was further purified by reverse phase column chromatography (120 g C18 cartridge, elution gradient 10%->60% acetonitrile/water). The pure fractions were pooled and freeze dried to afford compound 27 as an amorphous white solid (14.3 g, 20.5 mmol, 50%).

Compound 27 (14.3, 20.5 mmol) was dissolved in methanol (200 mL) and palladium on carbon (10 wt %, 2.18 g, 2.05 mmol) was added. The reaction was stirred under an atmosphere of hydrogen (1 atm) for 3 h. The mixture was filtered through Celite and concentrated to a semi solid. The residue was freeze dried to give Compound 28 as an amorphous white solid (12.1 g, 20.0 mmol, 98%). ¹H NMR (300 MHZ, dmso) δ 11.92 (s, 1H), 7.82 (d, J=9.2 Hz, 1H), 7.68 (t, J=5.9 Hz, 1H), 5.19 (d, J=3.4 Hz, 1H), 4.95 (dd, J=11.2, 3.4 Hz, 1H), 4.54 (d, J=8.5 Hz, 1H), 4.04-3.95 (m, 3H), 3.94-3.71 (m, 4H), 3.64-3.43 (m, 7H), 3.11-2.99 (m, 2H), 2.16 (t, J=7.3 Hz, 2H), 2.08 (s, 3H), 1.98 (s, 3H), 1.87 (s, 3H), 1.75 (s, 3H), 1.54-1.31 (m, 4H), 1.29-1.17 (m, 2H).

### Example 5—Synthesis and Characterisation of GalNAc Linker 7

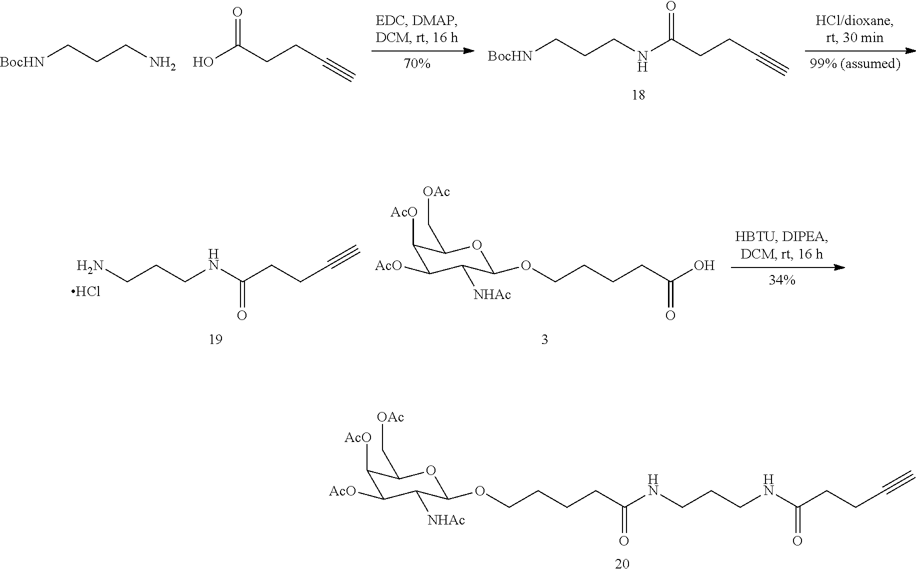

4-pentynoic acid (50 g, 0.510 mol), 1-ethyl-3-(3-dimethylaminopropyl) carbodiimide hydrochloride (117.4 g, 0.612 mol), and 4-(dimethylamino) pyridine (15.6 g, 0.128 mol) were dissolved in dichloromethane (1300 mL) at room temperature. Triethylamine (213 mL, 1.53 mol) was added to the reaction mixture, followed by N-Boc-1,3-propanediamine (88.8 g, 0.51 mol). The reaction was stirred at room temperature for 16 h, then poured into water (1.5 L). The layers were separated and the aqueous phase was extracted with dichloromethane (2×800 mL). The combined organic extracts were dried with sodium sulfate, filtered and concentrated under reduced pressure. The crude product was purified by column chromatography (2%->5% methanol/dichloromethane) to afford compound 18 (91.5 g, 0.360 mol, 71%) as a thick yellow oil.

Compound 18 (51.8 g, 0.204 mol) was dissolved in dichloromethane (500 mL) and hydrochloric acid (4.0 M in dioxane, 500 mL, 2.0 mol) was added. The reaction was stirred at room temperature for 30 min, then concentrated in vacuo to remove volatiles. The residue was washed with methyl tert-butyl ether (2×50 mL) to afford Compound 19 (43.6 g, 0.283 mol, 112%) as a thick oil. The product was dissolved in pyridine (204 mL) to make a 1.0 M solution of Compound 19 in pyridine.

Compound 3 (17.0 g, 38.0 mmol) was dissolved in dichloromethane (190 mL), then HBTU (14.4 g, 38.0 mmol), DIEA (20 mL, 114 mmol) and a solution of Compound 19 in pyridine (41.8 mL, 41.8 mmol) were added sequentially at room temperature. The reaction mixture was stirred at room temperature for 16 h, then poured into saturated aqueous NaHCO₃ (150 mL). The layers were separated and the organic phase was sequentially washed with 10% citric acid (100 mL) and brine (150 mL). The organic phase was dried with sodium sulfate, filtered and concentrated under reduced pressure. The crude product was purified by column chromatography (0%->10% methanol/dichloromethane) to afford Compound 20 (7.61 g, 13.0 mol, 71%) as a white foam.

¹H NMR (300 MHZ, dmso) δ 7.88 (t, J=5.6 Hz, 1H), 7.83 (d, J=9.2 Hz, 1H), 7.73 (t, J=5.7 Hz, 1H), 5.21 (d, J=3.4 Hz, 1H), 4.97 (dd, J=11.3, 3.3 Hz, 1H), 4.48 (dd, J=8.5, 1.1 Hz, 1H), 4.06-4.00 (m, 3H), 3.87 (q, J=9.4 Hz, 1H), 3.71 (dd, J=10.4, 5.4 Hz, 1H), 3.42 (q, J=5.5 Hz, 1H), 3.04 (q, J=6.4 Hz, 4H), 2.76 (td, J=2.5, 1.1 Hz, 1H), 2.40-2.30 (m, 2H), 2.30-2.19 (m, 2H), 2.11 (d, J=1.1 Hz, 3H), 2.03 (d, J=7.3 Hz, 2H), 2.00 (d, J=1.1 Hz, 3H), 1.89 (d, J=1.1 Hz, 3H), 1.78 (d, J=1.1 Hz, 3H), 1.58-1.39 (m, 6H).

### Example 6—Synthesis and Characterisation of GalNAc Linker 8

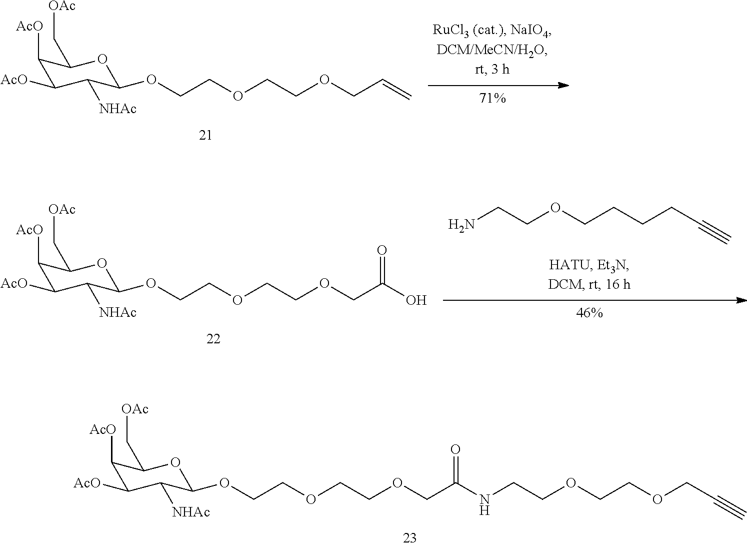

Compound 21 (4.85 g, 10.2 mmol) was dissolved in a mixture of dichloromethane (18 mL), acetonitrile (18 mL) and water (27 mL). Sodium metaperiodate (11.6 g, 54.1 mmol) and ruthenium (III) chloride (63.4 mg, 0.31 mmol) were added to the solution. The reaction was stirred for at room temperature for 3 h, using a water bath to control any exotherm. The reaction was filtered, washing the filter with acetonitrile (2 x 20 mL) and water (2×20 mL). The filtrate was concentrated in vacuo to remove volatiles, then the aqueous residue was acidified with aqueous hydrochloric acid (1.0 M) until the pH was 2 and extracted with dichloromethane (4×75 mL). The combined organic extracts were washed with aqueous sodium thiosulfate solution (100 mL), dried over sodium sulfate, filtered, and evaporated to give Compound 22 (3.57 g, 7.24 mmol, 71%) as a white foam that was used without further purification.

Compound 22 (10.0 g, 20.2 mmol) was dissolved in dichloromethane (100 mL), then HATU (8.47 g, 22.3 mmol), triethylamine (8.47 mL, 60.7 mmol) and alkynyl amine (2.90 g, 20.2 mmol) were added sequentially. The reaction was stirred at room temperature overnight, then concentrated to a residue. The crude product was purified by column chromatography (0%->10% methanol/(1:1 ethyl acetate: dichloromethane)) to obtain a white solid (7.4 g). This was further purified by reverse phase column chromatography (120 g C18 cartridge, elution gradient 10%->60% B acetonitrile/water. The pure fractions were pooled and freeze dried to afford Compound 23 as an amorphous white solid. (5.76 g, 9.31 mmol, 46%).

¹H NMR (300 MHZ, dmso) δ 7.83 (d, J=9.2 Hz, 1H), 7.69 (t, J=5.8 Hz, 1H), 5.22 (d, J=3.4 Hz, 1H), 4.98 (dd, J=11.2, 3.4 Hz, 1H), 4.56 (d, J=8.5 Hz, 1H), 4.14 (dd, J=2.4, 0.7 Hz, 2H), 4.06-4.00 (m, 3H), 3.95-3.75 (m, 3H), 3.65-3.50 (m, 11H), 3.47-3.40 (m, 3H), 3.32-3.21 (m, 3H), 2.11 (s, 3H), 2.00 (s, 3H), 1.90 (s, 3H), 1.78 (s, 3H).

### Example 7—Synthesis and Characterisation of GalNAc Linker 9

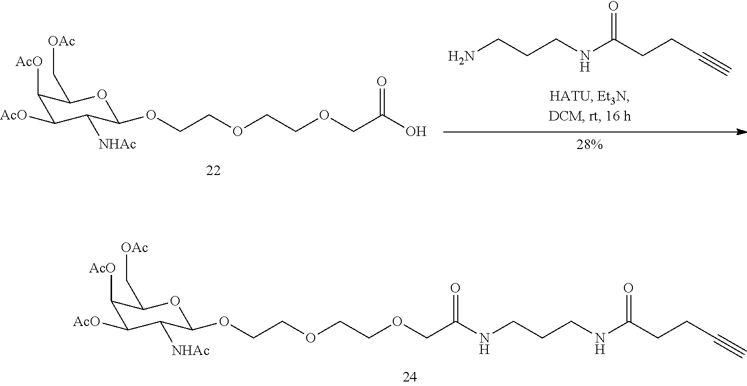 [SMILES: CC(=O)NC1C(OCCOCCOCC(=O)O)OC(COC(C)=O)C(OC(C)=O)C1OC(C)=O ; C#CCCC(=O)NCCCN ; C#CCCC(=O)NCCCNC(=O)COCCOCCOC1OC(COC(C)=O)C(OC(C)=O)C(OC(C)=O)C1NC(C)=O]

Compound 22 (10.3 g, 20.8 mmol) was dissolved in dichloromethane (100 mL), then HATU (8.71 g, 22.9 mmol), triethylamine (8.69 mL, 62.4 mmol) and alkynyl amine (3.21 g, 20.8 mmol) were added sequentially. The reaction was stirred at room temperature overnight, then concentrated to a residue. The crude product was purified by column chromatography (0%->10% methanol/(1:1 ethyl acetate: dichloromethane)) to obtain a white solid (4.8 g). This was further purified by reverse phase column chromatography (120 g C18 cartridge, elution gradient 10%->60% B acetonitrile/water. The pure fractions were pooled and freeze dried to afford Compound 24 as an amorphous white solid. (3.60 g, 5.72 mmol, 28%).

¹H NMR (300 MHZ, dmso) δ 7.89 (t, J=5.7 Hz, 1H), 7.84 (d, J=9.2 Hz, 1H), 7.74 (t, J=6.0 Hz, 1H), 5.21 (d, J=3.4 Hz, 1H), 4.97 (dd, J=11.2, 3.4 Hz, 1H), 4.56 (d, J=8.5 Hz, 1H), 4.07-3.99 (m, 3H), 3.95-3.73 (m, 4H), 3.66-3.46 (m, 7H), 3.17-2.98 (m, 4H), 2.76 (t, J=2.6 Hz, 1H), 2.41-2.31 (m, 2H), 2.31-2.19 (m, 2H), 2.10 (s, 3H), 2.00 (s, 3H), 1.89 (s, 3H), 1.77 (s, 3H), 1.53 (p, J=6.9 Hz, 2H).

### Example 8—Synthesis and Characterisation of GalNAc Linker 10

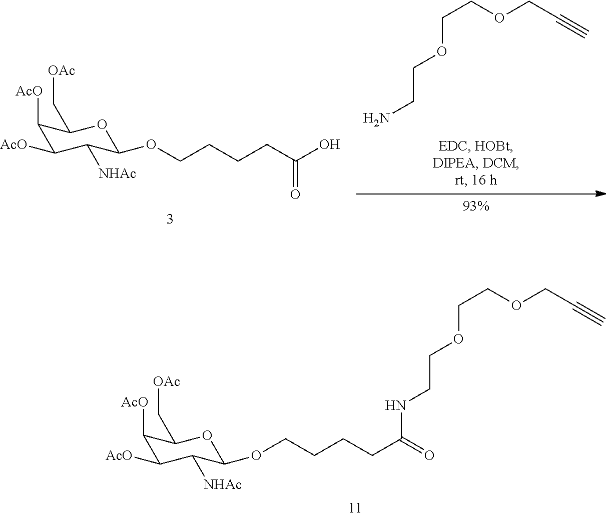

Compound 3 (28.2 g, 63.0 mmol) and 1-hydroxybenzotriazole hydrate (1.93 g, 12.6 mmol) were dissolved in dichloromethane (300 mL) and cooled to 0° C. To this solution, diisopropylethylamine (13.2 mL, 75.6 mmol), 2-[2-(2-propynyloxy) ethoxy] ethylamine (9.4 mL, 66.2 mmol) and 1-ethyl-3-(3-dimethylaminopropyl) carbodiimide hydrochloride (36.3 g, 189 mmol) were added sequentially, then the reaction mixture was stirred at room temperature for 16 h. The reaction mixture was cooled to 0° C. and quenched with saturated aqueous ammonium chloride (150 mL). The layers were separated and the aqueous phase was extracted with dichloromethane (3×150 mL). The combined organic extracts were dried with sodium sulfate, filtered and concentrated under reduced pressure. The crude product was purified by column chromatography (0%->5% methanol/dichloromethane) to afford Compound 11 (33.7 g, 58.8 mmol, 93%) as a thick yellow oil.

¹H NMR (400 MHZ, DMSO) δ 7.85-7.78 (m, 2H), 5.21 (d, J=3.4 Hz, 1H), 4.96 (dd, J=11.2, 3.4 Hz, 1H), 4.48 (d, J=8.4 Hz, 1H), 4.14 (d, J=2.4 Hz, 2H), 4.06-3.99 (m, 3H), 3.87 (dt, J=11.3, 8.9 Hz, 1H), 3.70 (dt, J=9.5, 5.6 Hz, 1H), 3.59-3.48 (m, 4H), 3.44-3.36 (m, 4H), 3.18 (q, J=5.8 Hz, 2H), 2.10 (s, 3H), 2.05 (t, J=7.0 Hz, 2H), 2.00 (s, 3H), 1.89 (s, 3H), 1.77 (s, 3H), 1.54-1.38 (m, 4H).

### Example 9-Synthesis and Characterisation of GalNAc monomer precursor 1

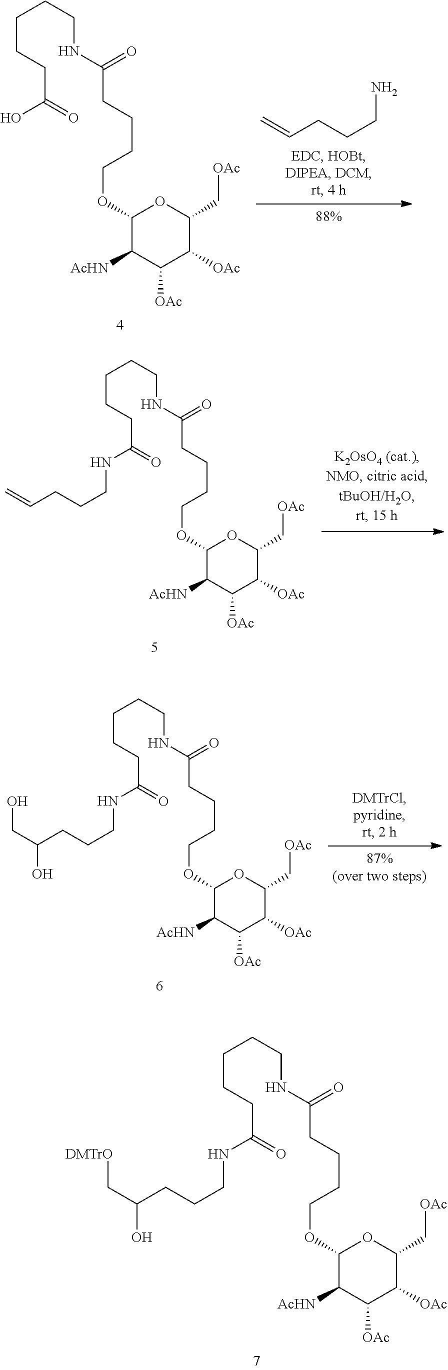

Compound 4 (5.0 g, 8.92 mmol) and 1-hydroxybenzotriazole hydrate (683 mg, 4.46 mmol) were dissolved in dichloromethane (80 mL), then the reaction mixture was cooled to 0° C. Diisopropylethylamine (3.4 mL, 19.6 mmol), 4-penten-1-amine hydrochloride (1.09 g, 8.92 mmol) and N-Ethyl-N′-(3-dimethylaminopropyl) carbodiimide hydrochloride (5.13g, 26.8 mmol) were added sequentially. The reaction mixture was stirred at room temperature for 15 h, then diluted with dichloromethane (120 mL) and quenched with aqueous ammonium chloride (60 mL). The layers were separated and the aqueous phase was extracted with dichloromethane (3×50 mL). The combined organic extracts were dried with sodium sulfate, filtered and concentrated under reduced pressure. The crude product was purified by column chromatography (3%->5%->10% methanol/dichloromethane) to afford Compound 5 (4.90 g, 7.81 mmol, 88%) as a white foam.

¹H NMR (400 MHZ, DMSO) δ 7.82 (d, J=9.3 Hz, 1H), 7.74 (t, J=5.6 Hz, 1H), 7.70 (t, J=5.6 Hz, 1H), 5.80 (ddt, J=16.9, 10.2, 6.6 Hz, 1H), 5.21 (d, J=3.4 Hz, 1H), 5.01 (ddt, J=17.2, 2.3, 1.6 Hz, 1H), 4.98-4.93 (m, 2H), 4.48 (d, J=8.5 Hz, 1H), 4.08-3.98 (m, 3H), 3.87 (dt, J=11.3, 8.9 Hz, 1H), 3.70 (dt, J=9.8, 5.7 Hz, 1H), 3.39 (dt, J=9.8, 6.1 Hz, 1H), 3.06-2.95 (m, 4H), 2.10 (s, 3H), 2.06-1.96 (m, 6H), 2.00 (s, 3H), 1.89 (s, 3H), 1.77 (s, 3H), 1.53-1.31 (m, 10H), 1.26-1.15 (m, 2H).

Compound 5 (3.00 g, 4.78 mmol) was dissolved in tert-butanol/water (1:1, 50 mL) and cooled to 0° C. Citric acid (918 mg, 4.78 mmol) and 4-methylmorpholine N-oxide (599 mg, 5.11 mmol) were added followed by potassium osmate (VI) dihydrate (18 mg, 0.048 mmol). The reaction was stirred at 0° C. for 2 h, then at room temperature for 14 h. The reaction mixture was concentrated under reduced pressure, then azeotroped with acetonitrile (4×40 mL) and dried under vacuum to afford Compound 6 which was used without further purification.

Compound 6 (3.16 g, 4.78 mmol, assumed) was azeotroped with pyridine (2×40 mL) and dissolved in pyridine (45 mL) before adding 4,4′-Dimethoxytriphenylmethyl chloride (1.70 g, 5.01 mmol) at 0° C. The reaction was stirred at room temperature for 1 h (1530-1630), then quenched with MeOH (1 mL) and concentrated under reduced pressure. The crude product was purified by column chromatography (2%->3%->4% methanol/dichloromethane) to give Compound 7 (4.00 g, 4.15 mmol, 87% over two steps) as a white foam.

¹H NMR (400 MHZ, DMSO) δ 7.82 (d, J=9.2 Hz, 1H), 7.74-7.66 (m, 2H), 7.44-7.36 (m, 2H), 7.34-7.17 (m, 7H), 6.92-6.84 (m, 4H), 5.21 (d, J=3.4 Hz, 1H), 4.96 (dd, J=11.2, 3.5 Hz, 1H), 4.66 (d, J=5.4 Hz, 1H), 4.48 (d, J=8.5 Hz, 1H), 4.08-3.97 (m, 3H), 3.87 (dt, J=11.3, 8.9 Hz, 1H), 3.73 (s, 6H), 3.74-3.65 (m, 1H), 3.64-3.54 (m, 1H), 3.45-3.36 (m, 1H), 3.04-2.94 (m, 4H), 2.90 (dd, J=9.0, 5.7 Hz, 1H), 2.73 (dd, J=9.0, 5.6 Hz, 1H), 2.10 (s, 3H), 2.06-1.96 (m, 4H), 1.99 (s, 3H), 1.89 (s, 3H), 1.77 (s, 3H), 1.55-1.14 (m, 14H).

### Example 10-Synthesis and Characterisation of GalNAc monomer precursor 2

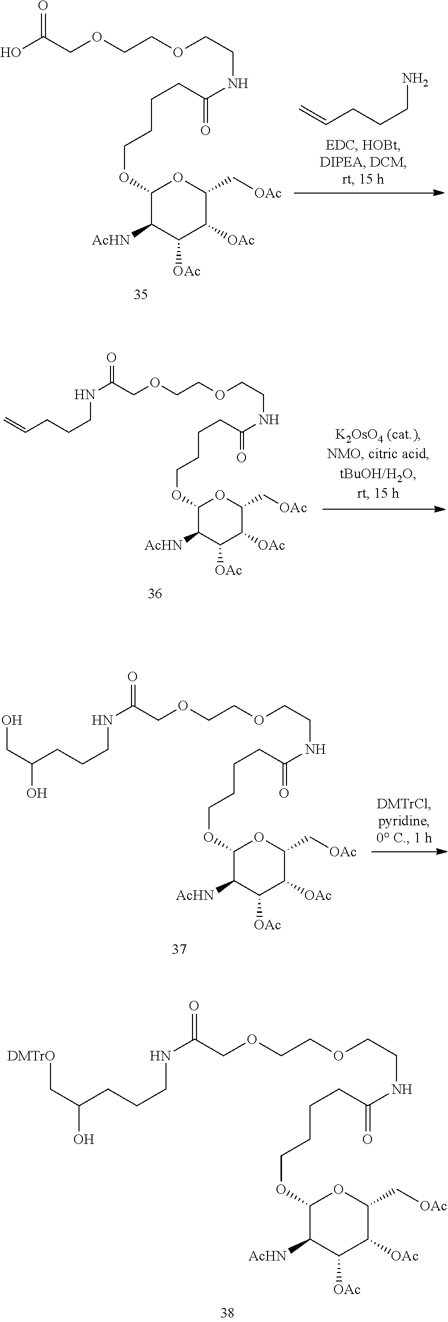

Compound 35 (3.0 g, 5.06 mmol) and amine (0.646 g, 5.32 mmol) were dissolved in dichloromethane (50 mL) and was cooled to 0° C. DIPEA (1.940 mL, 11.14 mmol), HOBt.H2O (0.388 g, 2.53 mmol) and EDC (2.911g, 15.19 mmol) were added sequentially. The reaction mixture was allowed to warm to rt and stirred for 2.5 h. The reaction mixture was then diluted with dichloromethane (50 mL) and quenched with aqueous NH₄Cl (50 mL). The layers were separated, and the aqueous phase was extracted with dichloromethane (3×50 mL). The combined organic extracts were dried with Na₂SO₄, filtered and concentrated under reduced pressure. The crude product was purified by column chromatography (3%->5%->10% MeOH/DCM) to afford Compound 36 (3.37g, 5.11 mmol, 100%) as a white foam.

1H NMR (400 MHZ, DMSO) δ 7.82 (ddt, J=16.9, 10.2, 6.6 Hz, 2H), 7.69 (t, J=6.0 Hz, 1H), 5.82 (dd, J=6.6, 0.1 Hz, 1H), 5.22 (d, J=3.4 Hz, 1H), 5.06-5.00 (m, 1H), 4.99-4.93 (m, 2H), 4.49 (d, J=8.5 Hz, 1H), 4.07-3.99 (m, 3H), 3.88 (dt, J=11.1, 8.9 Hz, 1H), 3.86 (s, 2H), 3.71 (dt, J=9.3, 5.6 Hz, 1H), 3.61-3.51 (m, 4H), 3.46-3.36 (m, 3H), 3.25-3.16 (m, 2H), 3.19-3.06 (m, 2H), 2.11 (s, 3H), 2.09-1.97 (m, 7H), 1.90 (s, 3H), 1.78 (s, 3H), 1.57-1.39 (m, 6H).

Compound 36 (3.34 g, 5.06 mmol) was dissolved in tBuOH/water (1:1, 50 mL) and cooled to 0° C. Citric acid (973 mg, 5.06 mmol) and NMO (635 mg, 5.42 mmol) were added followed by K2OsO4.2H20 (19 mg, 0.051 mmol). The reaction was warmed to rt and stirred over 15 h. The reaction mixture was extracted with aqueous NaHCO₃ (30 mL) followed by 25% methanol/dichloromethane mixture (50 mL×4) and was dried under vacuum for 2 h to afford Compound 37 which was used without further purification.

1H NMR (400 MHZ, DMSO) δ 7.84-7.77 (m, 2H), 7.65 (t, J=5.9 Hz, 1H), 5.22 (d, J=3.4 Hz, 1H), 4.97 (dd, J=11.3, 3.5 Hz, 1H), 4.49 (d, J=8.4 Hz, 1H), 4.43 (t, J=5.6 Hz, 1H), 4.40 (d, J=5.0 Hz, 1H), 4.08-3.98 (m, 3H), 3.92-3.82 (m, 3H), 3.71 (dt, J=9.7, 5.7 Hz, 1H), 3.61-3.51 (m, 4H), 3.46-3.37 (m, 4H), 3.30-3.17 (m, 4H), 3.10 (q, J=6.7 Hz, 2H), 2.11 (s, 3H), 2.07 (t, J=7.1 Hz, 2H), 2.00 (s, 3H), 1.90 (s, 3H), 1.78 (s, 3H), 1.61-1.35 (m, 8H).

Compound 37 (3.08 g, 4.44 mmol, assumed) was azeotroped with pyridine (2×30mL) and purged with argon. Compound 37 was then dissolved in pyridine (45 mL) and cooled to 0° C. before DMTrCI (1.58 g, 4.66 mmol) was added. The reaction was then allowed to warm to rt and stirred for 1 h. The reaction was quenched with methanol (5 mL) and concentrated under reduced pressure. The reaction was then extracted with NaHCO₃ (50mL) and washed with dichloromethane (3×50mL). The crude product was purified by column chromatography (0%->2%->3%->5% methanol/1% Triethylamine in dichloromethane) to afford Compound 38 (3.64g, 3.65 mmol, 82% over two steps) as a white foam.

1H NMR (400 MHZ, DMSO) δ 7.85-7.78 (m, 2H), 7.66 (t, J=5.9 Hz, 1H), 7.44-7.37 (m, 2H), 7.35-7.18 (m, 7H), 6.92-6.85 (m, 4H), 5.22 (d, J=3.5 Hz, 1H), 4.97 (dd, J=11.3, 3.5 Hz, 1H), 4.68 (d, J=5.4 Hz, 1H), 4.49 (d, J=8.5 Hz, 1H), 4.06-3.98 (m, 3H), 3.93-3.80 (m, 3H), 3.74 (s, 7H), 3.66-3.50 (m, 5H), 3.44-3.37 (m, 3H), 3.19 (q, J=5.8 Hz, 2H), 3.09 (q, J=6.5 Hz, 2H), 2.91 (dd, J=9.0, 5.6 Hz, 1H), 2.78-2.69 (m, 1H), 2.10 (s, 3H), 2.06 (t, J=7.1 Hz, 2H), 2.00 (s, 3H), 1.89 (s, 3H), 1.77 (s, 3H), 1.58-1.33 (m, 7H), 1.30-1.19 (m, 1H).

### Example 11—Synthesis and Characterisation of GalNAc Monomer Precursor 3

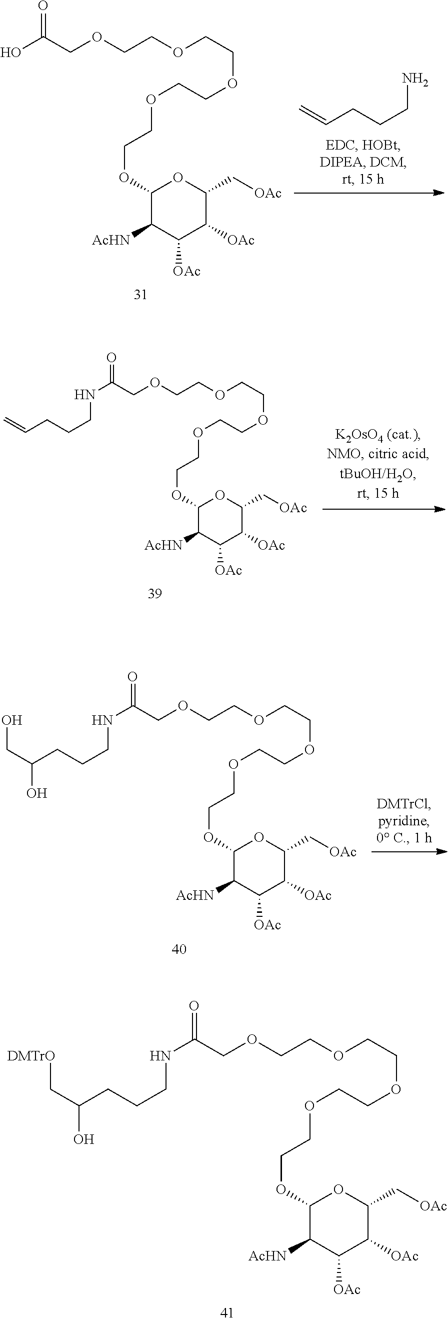

Compound 31 (4.1 g, 7.05 mmol) and amine (0.90 g, 7.40 mmol) were dissolved in dichloromethane (70 mL) and the reaction mixture was cooled to 0° C. DIPEA (2.70 mL, 15.51 mmol), HOBt.H2O (0.540 g, 3.52 mmol) and EDC (4.05g, 21.15 mmol) were then added sequentially. The reaction mixture was then cooled to 0° C. and diluted with dichloromethane (50mL) and quenched with aqueous NH₄Cl (50 mL) followed by extraction with dichloromethane (3×70 mL). The organic layers were then combined and dried with Na₂SO₄, filtered and concentrated under reduced pressure. The crude product was purified by column chromatography (3%->5%->10%->50% methanol/dichloromethane) to afford the compound 39 (4.48g, 6.91 mmol, 98%) as a white foam.

1H NMR (400 MHZ, DMSO) δ 7.79 (d, J=9.2 Hz, 1H), 7.70-7.63 (m, 1H), 5.81 (ddt, J=16.9, 10.2, 6.6 Hz, 1H), 5.22 (d, J=3.4 Hz, 1H), 5.07-4.92 (m, 3H), 4.56 (d, J=8.5 Hz, 1H), 4.07-4.00 (m, 3H), 3.93-3.84 (m, 3H), 3.78 (ddd, J=10.9, 5.4, 3.9 Hz, 1H), 3.65-3.45 (m, 15H), 3.16-3.07 (m, 2H), 2.11 (s, 3H), 2.05-1.97 (m, 5H), 1.90 (s, 3H), 1.78 (s, 3H), 1.57-1.45 (m, 2H).

Compound 39 (4.48 g, 6.91 mmol) was dissolved in tBuOH/water (1:1, 70 mL) and cooled to 0° C. Citric acid (1.327g, 6.91 mmol) and NMO (0.866g, 7.39 mmol) were added followed by K2OsO4.2H20 (25 mg, 0.069 mmol). The reaction was warmed to rt and stirred over 15 h. The reaction mixture was extracted with aqueous NaHCO₃ (40 mL) followed by 25% methanol/dichloromethane mixture (50 mL×4) and was dried under vacuum for 2 h to afford Compound 40 which was used without further purification.

1H NMR (400 MHZ, DMSO) δ 7.80 (d, J=9.2 Hz, 1H), 7.66 (t, J=5.9 Hz, 1H), 5.22 (d, J=3.4 Hz, 1H), 4.98 (dd, J=11.2, 3.5 Hz, 1H), 4.56 (d, J=8.5 Hz, 1H), 4.45 (t, J=5.5 Hz, 1H), 4.41 (d, J=4.9 Hz, 1H), 4.07-4.00 (m, 3H), 3.90 (dt, J=11.1, 8.9 Hz, 1H), 3.86 (s, 2H), 3.79 (ddd, J=10.9, 5.4, 3.9 Hz, 1H), 3.62-3.46 (m, 15H), 3.42-3.35 (m, 1H), 3.30-3.18 (m, 2H), 3.10 (q, J=6.7 Hz, 2H), 2.11 (s, 3H), 2.01 (s, 3H), 1.90 (s, 3H), 1.78 (s, 3H), 1.61-1.49 (m, 1H), 1.49-1.34 (m, 2H), 1.27-1.12 (m, 1H).

Compound 40 (4.79 g, 7.02 mmol, assumed) was azeotroped with pyridine (10mL) and purged with argon. Compound 40 was then dissolved in pyridine (10 mL) and cooled to 0° C. before DMTrCI (2.50 g, 7.37 mmol) was added. The reaction was then allowed to warm to rt and stirred for 1 h. The reaction was quenched with methanol (5 mL) and concentrated under reduced pressure. The reaction was then extracted with NaHCO₃ (50mL) and washed with dichloromethane (3×50mL). The crude product was purified by column chromatography (0%->2%->3%->5% methanol/1% Triethylamine in dichloromethane) to afford compound 41 (4.06g, 4.12 mmol, 58.7% over two steps) as a white foam.

¹H NMR (400 MHZ, DMSO) δ 7.80 (d, J=9.2 Hz, 1H), 7.65 (t, J=5.9 Hz, 1H), 7.44-7.37 (m, 2H), 7.35 - 7.16 (m, 7H), 6.93-6.84 (m, 4H), 5.22 (d, J=3.4 Hz, 1H), 4.98 (dd, J=11.2, 3.4 Hz, 1H), 4.67 (d, J=5.3 Hz, 1H), 4.56 (d, J=8.5 Hz, 1H), 4.08-3.99 (m, 3H), 3.94-3.87 (m, 1H), 3.85 (s, 2H), 3.83-3.70 (m, 7H), 3.63-3.43 (m, 16H), 3.09 (q, J=6.3 Hz, 2H), 2.91 (dd, J=9.0, 5.6 Hz, 1H), 2.74 (dd, J=9.0, 5.7 Hz, 1H), 2.11 (s, 3H), 2.00 (s, 3H), 1.90 (s, 3H), 1.78 (s, 3H), 1.56-1.44 (m, 2H), 1.43-1.32 (m, 1H), 1.30-1.19 (m, 1H).

### Example 12-Synthesis and Characterisation of GalNAc monomer precursor 4

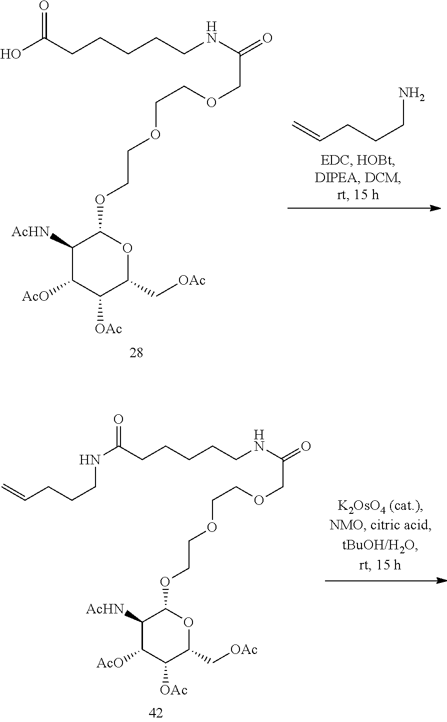

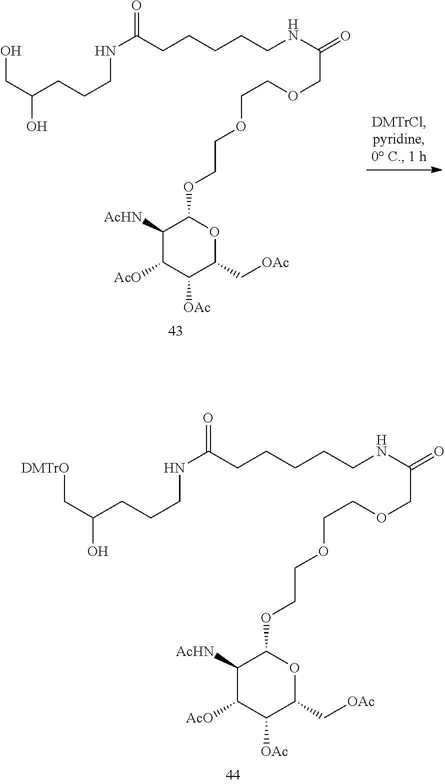

Compound 28 (2.3 g, 3.79 mmol) and amine (0.484 g, 3.98 mmol) was dissolved in dichloromethane (40 mL) and the reaction mixture was cooled to 0° C. DIPEA (1.45 mL, 8.34 mmol), HOBt.H2O (0.29 g, 1.90 mmol) and EDC (2.18g, 11.37 mmol) were then added sequentially. The reaction mixture was then allowed to warm to room temperature and stirred for 15 h. The reaction mixture was then cooled to 0° C. and diluted with dichloromethane (40mL) and quenched with aqueous NH₄Cl (40 mL) followed by extraction with dichloromethane (3×40 mL). The organic layers were then combined and dried with Na₂SO₄, filtered and concentrated under reduced pressure. The crude product was purified by column chromatography (3%->5%->10% MeOH/DCM) to afford the compound 42 (2.77g, 4.11 mmol, 99.8%) as an orange liquid.

¹H NMR (400 MHZ, DMSO) δ 7.83 (d, J=9.2 Hz, 1H), 7.75 (t, J=5.6 Hz, 1H), 7.67 (t, J=5.9 Hz, 1H), 5.80 (ddt, J=16.9, 10.2, 6.6 Hz, 1H), 5.22 (d, J=3.4 Hz, 1H), 5.07-4.92 (m, 3H), 4.57 (d, J=8.5 Hz, 1H), 4.09-3.99 (m, 3H), 3.95-3.86 (m, 1H), 3.85 (s, 2H), 3.80 (ddd, J=10.3, 5.1, 3.4 Hz, 1H), 3.66-3.48 (m, 7H), 3.13-2.98 (m, 4H), 2.11 (s, 3H), 2.07-1.97 (m, 7H), 1.90 (s, 3H), 1.78 (s, 3H), 1.45 (ddt, J=20.4, 14.8, 7.1 Hz, 6H), 1.27-1.15 (m, 2H).

Compound 42 (1.85 g, 2.75 mmol) was dissolved in tBuOH/water (1:1, 15 mL) and cooled to 0° C. Citric acid (528 mg, 2.75 mmol) and NMO (345 mg, 2.94 mmol) were added followed by K2OsO4.2H20 (10 mg, 0.027 mmol). The reaction was stirred at 0° C. and slowly warmed to rt over 15 h. The reaction mixture was concentrated under reduced pressure, then azeotroped with acetonitrile (4×40 mL) and dried under vacuum for 2 h to afford Compound 43 which was used without further purification.

Compound 43 (1.12 g, 1.59 mmol, assumed) was azeotroped with pyridine (2×30 mL) and purged with argon. Compound 43 was then dissolved in pyridine (30 mL) and cooled to 0° C. before adding DMTrCI (0.56 g, 1.67 mmol). The reaction was stirred at rt for 1 h. The reaction was quenched with methanol (1 mL) and concentrated under reduced pressure and dried in vacuo overnight. The crude product was purified by column chromatography (0%->2%->3%->5% MeOH/DCM) to give Compound 44 (0.81g, 0.80 mmol, 29% over two steps) as a white foam.

1H NMR (400 MHZ, DMSO) δ 7.83 (d, J=9.2 Hz, 1H), 7.71 (t, J=5.6 Hz, 1H), 7.66 (t, J=5.9 Hz, 1H), 7.45-7.37 (m, 2H), 7.35-7.18 (m, 7H), 6.93-6.84 (m, 4H), 5.22 (d, J=3.4 Hz, 1H), 4.99 (dd, J=11.2, 3.4 Hz, 1H), 4.67 (d, J=5.4 Hz, 1H), 4.57 (d, J=8.5 Hz, 1H), 4.08-3.99 (m, 3H), 3.90 (dt, J=11.0, 8.8

Hz, 1H), 3.85 (s, 2H), 3.80 (ddd, J=10.6, 5.3, 3.8 Hz, 1H), 3.74 (s, 6H), 3.65-3.48 (m, 8H), 3.07 (q, J=6.8 Hz, 2H), 3.00 (q, J=6.3 Hz, 2H), 2.91 (dd, J=9.0, 5.7 Hz, 1H), 2.74 (dd, J=9.0, 5.6 Hz, 1H), 2.11 (s, 3H), 2.06-1.98 (m, 5H), 1.90 (s, 3H), 1.78 (s, 3H), 1.53-1.15 (m, 10H).

### Example 13-Synthesis and Characterisation of GalNAc Monomer Precursor 5

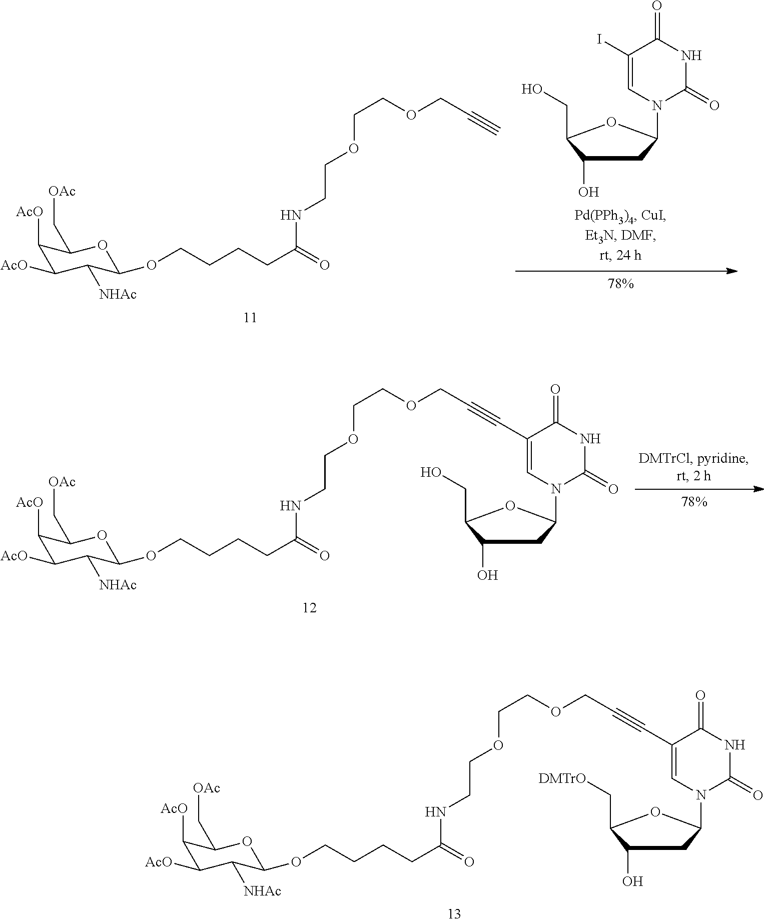

Compound 11 (29.4 g, 51.4 mmol) and 5-iododeoxyuridine (14.0 g, 39.5 mmol) were dissolved in N,N-dimethylformamide (160 mL) and degassed by sparging with argon for 30 min. Triethylamine (27.6 mL, 114 mmol) was added and the degassing was resumed for 10 min. Copper (I) iodide (753 mg, 3.95 mmol) and tetrakis (triphenylphosphine) palladium (0) (1.83 g, 1.58 mmol) were added sequentially, then the reaction was stirred under argon for 24 h at room temperature. The solvent was removed under reduced pressure and the crude product was purified by column chromatography (0->7%->10% methanol/dichloromethane) to give Compound 12 (24.8 g, 31.0 mmol, 78%) as an amber foam.

1H NMR (400 MHZ, DMSO) δ 11.64 (s, 1H), 8.25 (s, 1H), 7.87-7.78 (m, 2H), 6.11 (t, J=6.6 Hz, 1H), 5.24 (d, J=4.3 Hz, 1H), 5.21 (d, J=3.4 Hz, 1H), 5.12 (t, J=5.0 Hz, 1H), 4.96 (dd, J=11.2, 3.4 Hz, 1H), 4.48 (d, J=8.4 Hz, 1H), 4.33 (s, 2H), 4.27-4.19 (m, 1H), 4.07-3.98 (m, 3H), 3.87 (dt, J=11.2, 8.9 Hz, 1H), 3.80 (q, J=3.3 Hz, 1H), 3.70 (dt, J=9.5, 5.7 Hz, 1H), 3.65-3.50 (m, 6H), 3.45-3.36 (m, 3H), 3.22-3.15 (m, 2H), 2.16-2.11 (m, 2H), 2.10 (s, 3H), 2.05 (t, J=7.0 Hz, 2H), 2.00 (s, 3H), 1.89 (s, 3H), 1.77 (s, 3H), 1.54-1.39 (m, 4H).

Compound 12 (26.8 g, 33.6 mmol) was azeotroped with pyridine (2×50 mL) and dissolved in pyridine (150 mL) under an atmosphere of argon. The solution was cooled to 0°° C. before adding 4,4-dimethoxytrityl chloride (13.6 g, 40.3 mmol). The reaction was stirred at room temperature for 1 h, then quenched with MeOH (20 mL) and concentrated under reduced pressure. The crude product was purified by column chromatography (2%->5% methanol/dichloromethane) to give Compound 13 (29.1 g, 26.4 mmol, 79%) as a white foam.

1H NMR (400 MHZ, DMSO) δ 11.63 (s, 1H), 7.98 (s, 1H), 7.84-7.76 (m, 2H), 7.43-7.35 (m, 2H), 7.35-7.26 (m, 6H), 7.26-7.18 (m, 1H), 6.93-6.84 (m, 4H), 6.11 (t, J=6.6 Hz, 1H), 5.33 (d, J=4.5 Hz, 1H), 5.21 (d, J=3.4 Hz, 1H), 4.96 (dd, J=11.3, 3.4 Hz, 1H), 4.48 (d, J=8.5 Hz, 1H), 4.30-4.22 (m, 1H), 4.13 (d, J=16.1 Hz, 1H), 4.08 (d, J=16.0 Hz, 1H), 4.05-3.99 (m, 3H), 3.94-3.89 (m, 1H), 3.86 (dt, J=11.1, 8.8 Hz, 1H), 3.73 (s, 6H), 3.73-3.66 (m, 1H), 3.46-3.32 (m, 7H), 3.26-3.08 (m, 4H), 2.28 (dt, J=13.3, 6.6 Hz, 1H), 2.19 (ddd, J=13.4, 6.3, 3.6 Hz, 1H), 2.10 (s, 3H), 2.05 (t, J=7.0 Hz, 2H), 1.99 (s, 3H), 1.89 (s, 3H), 1.77 (s, 3H), 1.54-1.37 (m, 4H).

### Example 14—Synthesis and Characterisation of GalNAc Monomer Precursor 6

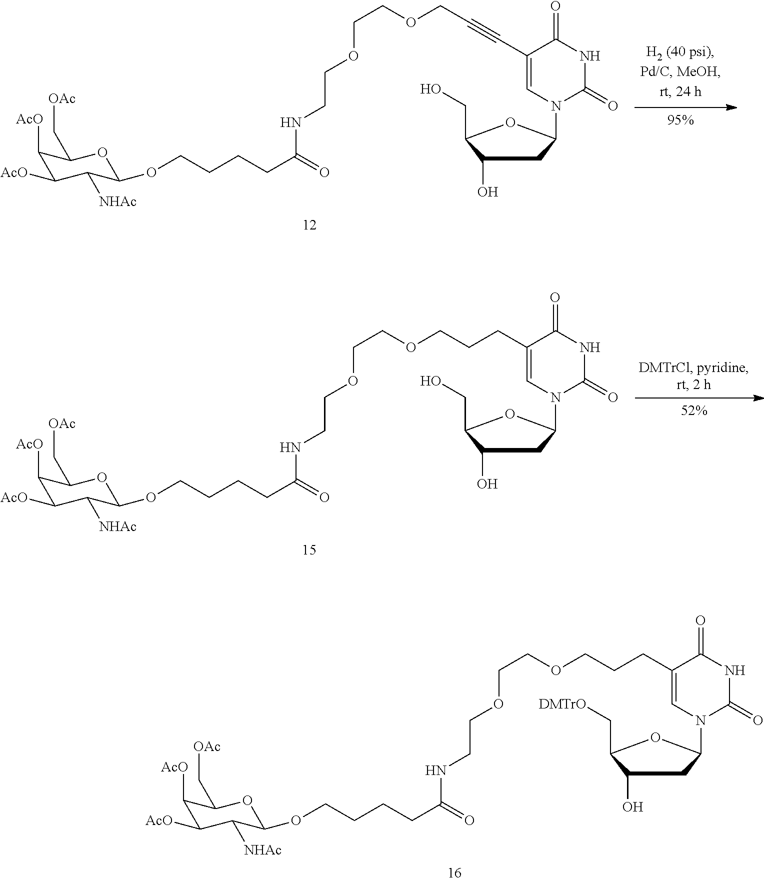

Compound 12 (1.57 g, 1.97 mmol) was dissolved in MeOH (20 mL) followed by the addition of Pd/C (10 wt %, 628 mg, 0.590 mmol). The reaction was stirred under an atmosphere of hydrogen (40 psi) at room temperature for 24 h. The reaction mixture was filtered through a pad of Celite and concentrated under reduced pressure to give Compound 15 (1.50 g, 1.87 mmol, 95%) as a yellow foam that was used without further purification.

¹H NMR (300 MHZ, DMSO) δ 11.29 (s, 1H), 7.88-7.79 (m, 2H), 7.68 (s, 1H), 6.17 (t, J=6.8 Hz, 1H), 5.26 (dd, J=4.2, 0.9 Hz, 1H), 5.21 (d, J=3.4 Hz, 1H), 5.05 (t, J=5.0 Hz, 1H), 4.96 (dd, J=11.2, 3.4 Hz, 1H), 4.47 (d, J=8.5 Hz, 1H), 4.27-4.21 (m, 1H), 4.05-3.99 (m, 3H), 3.97-3.81 (m, 1H), 3.80-3.65 (m, 2H), 3.64-3.28 (m, 10H), 3.22-3.12 (m, 3H), 2.28-2.13 (m, 2H), 2.13-2.02 (m, 7H), 2.00 (s, 3H), 1.89 (s, 3H), 1.77 (s, 3H), 1.74-1.57 (m, 2H), 1.55-1.35 (m, 4H).

Compound 15 (1.40 g, 1.74 mmol) was azeotroped with pyridine (2×10 mL) and dissolved in pyridine (10 mL) under an atmosphere of argon. The solution was cooled to 0° C. before adding 4,4-dimethoxytrityl chloride (620 mg, 1.83 mmol). The reaction was stirred at room temperature for 1 h, then quenched with MeOH (2 mL) and concentrated under reduced pressure. The crude product was purified by column chromatography (2%->5% methanol/dichloromethane) to give Compound 16 (1.0 g, 0.905 mmol, 52%) as a white foam.

1H NMR (400 MHZ, DMSO) δ 11.33 (s, 1H), 7.84-7.74 (m, 2H), 7.42-7.36 (m, 3H), 7.35-7.28 (m, 2H), 7.27-7.20 (m, 5H), 6.93-6.84 (m, 4H), 6.20 (t, J=6.8 Hz, 1H), 5.33 (d, J=4.6 Hz, 1H), 5.21 (d, J=3.4 Hz, 1H), 4.96 (dd, J=11.3, 3.4 Hz, 1H), 4.48 (d, J=8.4 Hz, 1H), 4.30 (dq, J=7.9, 4.1 Hz, 1H), 4.07-3.97 (m, 3H), 3.93-3.81 (m, 2H), 3.78-3.65 (m, 7H), 3.46-3.35 (m, 7H), 3.23-3.10 (m, 6H), 2.29-2.20 (m, 1H), 2.20-2.12 (m, 1H), 2.10 (s, 3H), 2.04 (t, J=7.1 Hz, 2H), 1.99 (s, 3H), 1.97-1.81 (m, 5H), 1.77 (s, 3H), 1.54-1.37 (m, 6H).

### Example 15—Synthesis and Characterisation of GalNAc Amidite Monomer 3 (ptGal Phosphoramidite Monomer)

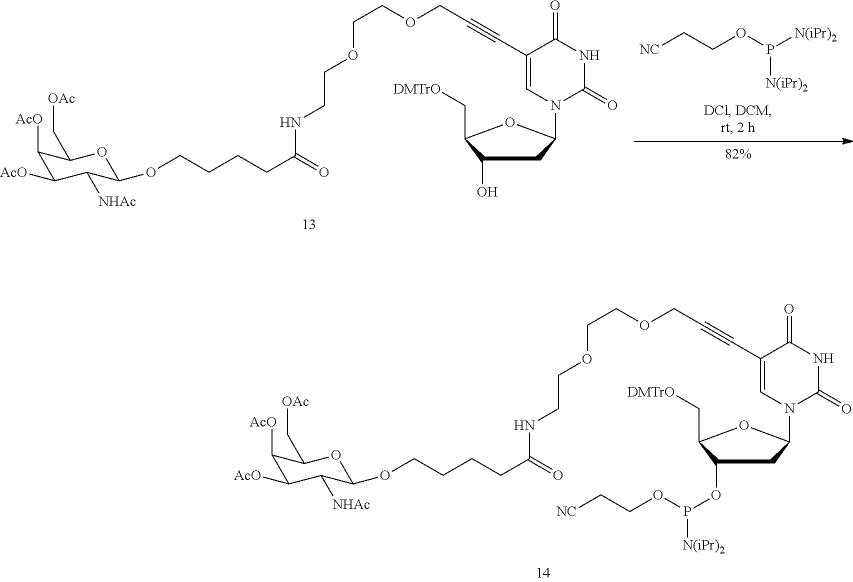

Compound 13 (20.8 g, 18.9 mmol) was azeotroped with dichloromethane (2×50 mL) and dried overnight, then dissolved in anhydrous dichloromethane (190 mL) and cooled to 0° C. 2-Cyanoethyl N,N,N′,N′-tetraisopropylphosphorodiamidite (7.2 mL, 22.7 mmol) and 4,5-dicyanoimidazole (2.23 g, 18.9 mmol) were added to the reaction mixture and stirred at 0° C. for 2 h. The reaction was diluted with dichloromethane (500 mL) and successively washed with aqueous sodium bicarbonate (2×100 mL) and brine (1×100 mL), then dried with sodium sulfate, filtered and concentrated under reduced pressure. The crude product was purified by column chromatography (0%->5%->7% methanol/ethyl acetate) to afford Compound 14-ptGal phosphoramidite monomer (20.2 g, 15.5 mmol, 82%) as a white foam.

¹H NMR (400 MHZ, DMSO) δ 11.71 (s, 1H), 8.02 (d, J=1.2 Hz, 1H), 7.84-7.78 (m, 2H), 7.44-7.36 (m, 2H), 7.36-7.18 (m, 7H), 6.92-6.83 (m, 4H), 6.10 (dt, J=9.1, 6.7 Hz, 1H), 5.21 (d, J=3.4 Hz, 1H), 4.96 (dd, J=11.2, 3.5 Hz, 1H), 4.51-4.45 (m, 1H), 4.20-4.06 (m, 2H), 4.06-3.98 (m, 5H), 3.87 (dt, J=11.3, 8.9 Hz, 1H), 3.78-3.65 (m, 8H), 3.65-3.44 (m, 3H), 3.44-3.33 (m, 8H), 3.29-3.22 (m, 1H), 3.21-3.12 (m, 2H), 2.76 (t, J=5.9 Hz, 1H), 2.64 (t, J=5.9 Hz, 1H), 2.47-2.28 (m, 2H), 2.10 (s, 3H), 2.05 (t, J=7.0 Hz, 2H), 2.01-1.97 (m, 3H), 1.89 (s, 3H), 1.77 (s, 3H), 1.53-1.38 (m, 4H), 1.15-1.06 (m, 9H), 0.97 (d, J=6.8 Hz, 3H). 31P NMR (162 MHZ, DMSO) δ 147.54, 147.15.

### Example 16—Synthesis and Characterisation of GalNAc Amidite Monomer 5 (tGal Phosphoramidite Monomer)

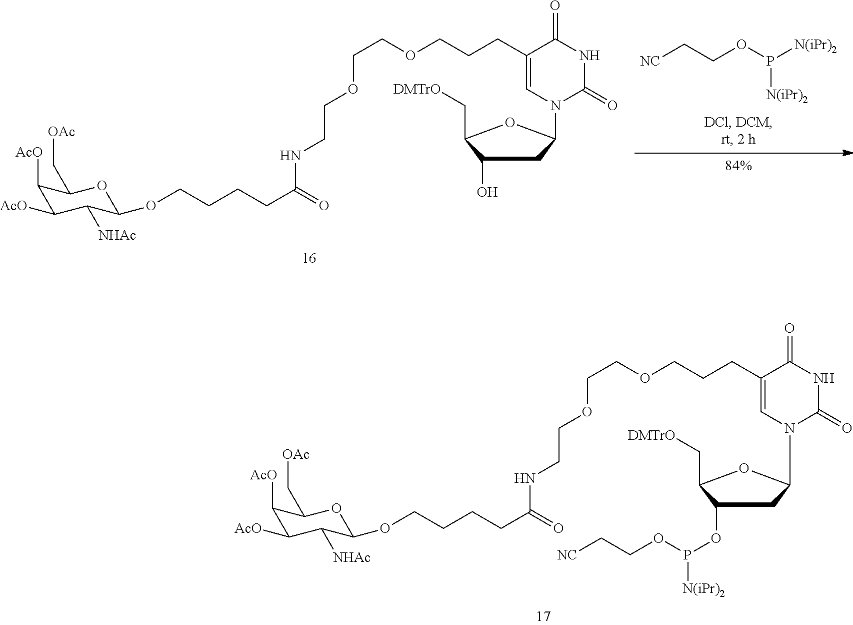

Compound 16 (1.0 g, 0.905 mmol) was azeotroped with dichloromethane (2×10 mL) and dried overnight, then dissolved in anhydrous dichloromethane (10 mL) and cooled to 0° C. 2-Cyanoethyl N,N,N′, N′-tetraisopropylphosphorodiamidite (0.34 mL, 1.09 mmol) and 4,5-dicyanoimidazole (107 mg, 0.905 mmol) were added to the reaction mixture and stirred at 0° C. for 2 h. The reaction mixture was diluted with dichloromethane (70 mL) and successively washed with aqueous sodium bicarbonate (2×15 mL) and brine (1×15 mL), then dried with sodium sulfate, filtered and concentrated under reduced pressure. The crude product was purified by column chromatography (2%->5% methanol/ethyl acetate) to afford Compound 17-tGal phosphoramidite monomer (1.0 g, 0.766 mmol, 84%) as a white foam.

1H NMR (400 MHZ, DMSO) δ 11.35 (s, 1H), 7.88-7.72 (m, 2H), 7.45-7.35 (m, 3H), 7.34-7.27 (m, 2H), 7.27-7.20 (m, 5H), 6.92-6.83 (m, 4H), 6.19 (td, J=6.8, 3.3 Hz, 1H), 5.21 (d, J=3.4 Hz, 1H), 4.97 (dd, J=11.2, 3.4 Hz, 1H), 4.58-4.50 (m, 1H), 4.48 (d, J=8.5 Hz, 1H), 4.08-3.96 (m, 4H), 3.87 (dt, J=11.1, 8.9 Hz, 1H), 3.79-3.65 (m, 8H), 3.65-3.45 (m, 4H), 3.44-3.38 (m, 4H), 3.31-3.18 (m, 4H), 3.20-3.11 (m, 4H), 2.76 (t, J=5.9 Hz, 1H), 2.64 (t, J=5.9 Hz, 1H), 2.43-2.23 (m, 2H), 2.10 (s, 3H), 2.04 (t, J=7.1 Hz, 2H), 2.01-1.91 (m, 5H), 1.89 (s, 3H), 1.77 (s, 3H), 1.45 (d, J=6.2 Hz, 6H), 1.15-1.06 (m, 9H), 0.97 (d, J=6.8 Hz, 3H).

### Example 17—Synthesis and Characterisation of GalNAc Amidite Monomer 6 (gGal Phosphoramidite Monomer)

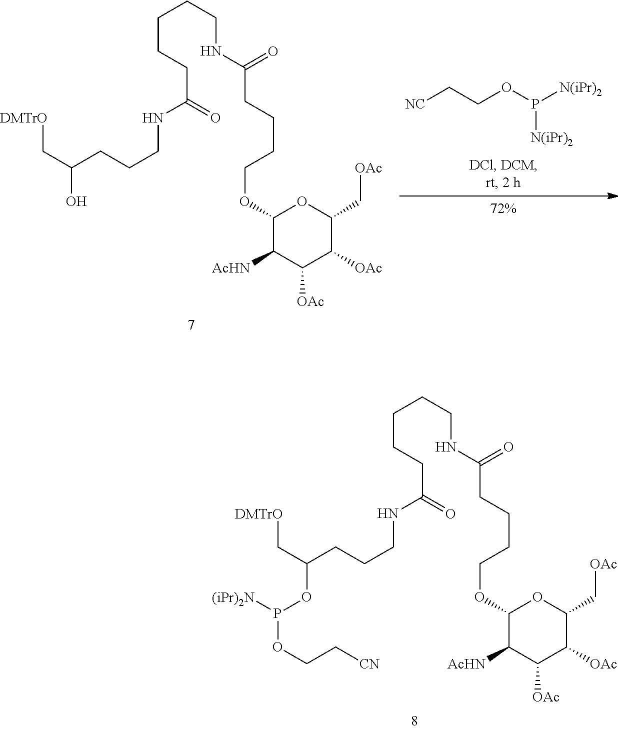

Compound 7 (1.85 g, 1.92 mmol) was azeotroped with acetonitrile (2×20 mL), then dissolved in anhydrous dichloromethane (20 mL) and cooled to 0° C. 2-Cyanoethyl N,N,N′,N′-tetraisopropylphosphorodiamidite (868 mg, 2.88 mmol) was added at this temperature, followed by 4,5-dicyanoimidazole (227 mg, 1.92 mmol). The reaction was stirred at 0° C. for 1.5 h, then diluted with dichloromethane (40 mL) and quenched with aqueous sodium bicarbonate (10 mL). The layers were separated and the aqueous phase was extracted with dichloromethane (3×20 mL). The combined organic extracts were dried with sodium sulfate, filtered and concentrated under reduced pressure. The crude product was purified by column chromatography (3%->5% methanol/ethyl acetate) to afford compound 8-gGal phosphoramidite monomer (1.6 g, 1.37 mmol, 72%) as a white foam.

1H NMR (400 MHZ, DMSO) δ 7.82 (d, J=9.2 Hz, 1H), 7.75-7.65 (m, 2H), 7.44-7.36 (m, 2H), 7.35-7.17 (m, 7H), 6.92-6.82 (m, 4H), 5.21 (d, J=3.4 Hz, 1H), 4.96 (dd, J=11.2, 3.5 Hz, 1H), 4.48 (d, J=8.5 Hz, 1H), 4.05-3.98 (m, 3H), 3.97-3.82 (m, 2H), 3.77-3.47 (m, 10H), 3.41 (dd, J=11.1, 5.1 Hz, 2H), 3.10-2.78 (m, 6H), 2.75 (t, J=5.9 Hz, 1H), 2.58 (dt, J=6.8, 5.1 Hz, 1H), 2.10 (s, 3H), 2.05-1.96 (m, 7H), 1.89 (s, 3H), 1.77 (s, 3H), 1.53-1.28 (m, 12H), 1.24-1.03 (m, 11H), 1.00 (d, J=6.7 Hz, 3H).

31P NMR (162 MHZ, DMSO) δ 147.53, 147.51.

### Example 18—Synthesis and Characterisation of GalNAc Solid Support 1 (gGal CPG)

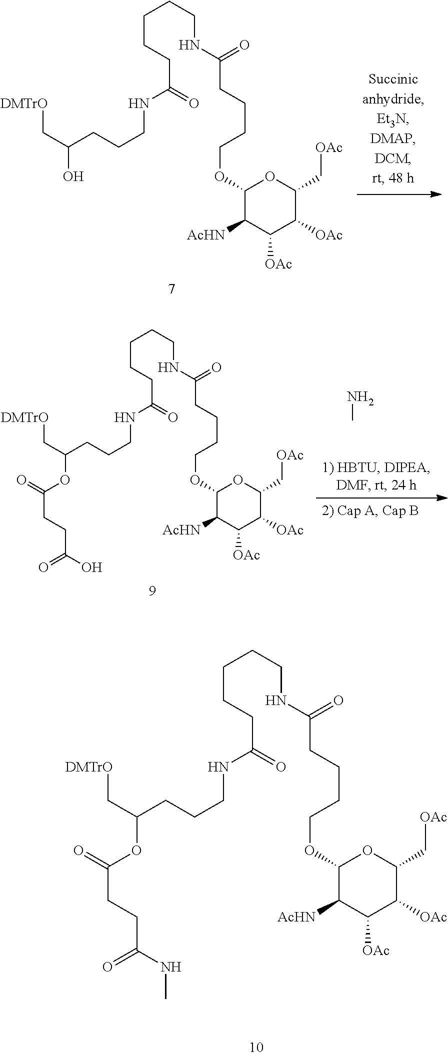

Compound 7 (350 mg, 0.363 mmol) was dissolved in dichloromethane (7 mL) at room temperature, then succinic anhydride (73 mg, 0.726 mmol), 4-dimethylaminopyridine (89 mg, 0.726 mmol) and triethylamine (0.3 mL, 2.18 mmol) were added successively. The reaction was stirred at room temperature for 64 h, then concentrated in vacuo to dryness. The crude product was purified by column chromatography (0->20% methanol/dichloromethane) to afford Compound 9 (386 mg, 0.363 mmol) as a white foam. Compound 9 (386 mg, 0.363 mmol) was dissolved in N,N-dimethylformamide (10 mL) at room temperature, then LCAA CPG (2.7 g, 0.221 mmol, amino content 82 umol/g), N,N-diisopropylethylamine (0.116 mL, 0.664 mmol) and N,N,N′, N′-Tetramethyl-O-(1H-benzotriazol-1-yl) uronium hexafluorophosphate (126 mg, 0.332 mmol) were added to the reaction mixture. The reaction was left to stand at room temperature for 24 h, with occasional gentle swirling to ensure homogeneity. The CPG was collected by filtration and washed on the filter with dichloromethane (3×10 mL), then dried in vacuo for 16 h.

The CPG was treated with a mixture of Cap A (acetic Anhydride in tetrahydrofuran and pyridine, 5 mL) and Cap B (N-methylimidazole in acetonitrile and pyridine, 5 mL) and left to stand at room temperature for 2 h, with occasional gentle swirling to ensure homogeneity. The gGal CPG (compound 10) was collected by filtration and washed on the filter with dichloromethane (5×10 mL), then dried in vacuo for 16 h.

### Example 19—Synthesis and Characterisation of GalNAc Conjugated Oligonucleotide 1 ((gGal)3-oligo) and GalNAc Conjugated siRNA 1 ((gGal)3-siRNA)

Antisense strands were synthesized on a MerMade 48 synthesizer using commercially available 5′-O-(4,4′-dimethoxytrityl)-2′-deoxy-, 5′-O-(4,4′-dimethoxytrityl)-2′- deoxy-2′-fluoro-, and 5′-O-(4,4′-dimethoxytrityl)-2′-O-methyl-3′-O-(2-cyanoethyl-N,N-diisopropyl) phosphoramidite monomers of uridine, thymidine, 4-N-acetylcytidine, 6-N-benzoyladenosine, and 2-N-isobutyrylguanosine using standard solid-phase oligonucleotide synthesis and deprotection protocols. In sense strand synthesis, respective GalNAc CPG and GalNAc amidite monomers were used. The antisense strand and sense strand are those of inclisiran, corresponding to SEQ ID NO: 4 and 8, respectively.

Inclisiran is a synthetic siRNA directed against PCSK9, and is described e.g. in Fitzgerald et al., NEJM (2017) 376 (1): 41-51 and WO 2014/089313 A1 (both of which are hereby incorporated by reference in their entirety). The sense strand of the siRNA is conjugated with a triantennary N-acetylgalactosamine (triGaINAc) ligand. The inhibitory nucleic acid inclisiran (brand name: Leqvio®) inhibits gene expression of PCSK9 and is approved in the EU for the treatment of dyslipidemias and hypercholesterolemia.

Solid phase synthesis on the gGal CPG was first introduced to couple with gGal phosphoramidite in two reaction cycles successively and then followed with standard oligonucleotide synthesis. Phosphorothioate linkages were introduced by oxidation of phosphite utilizing 0.1 M DDTT in pyridine. After cleavage and deprotection with aqueous methylamine at 55° C. for 4 hours, the (gGal)3-oligo (GalNAc conjugated oligonucleotide 1) was achieved in crude state and was further purified by by anion-exchange high-performance liquid chromatography (IEX-HPLC). The pure fractions were combined, concentrated, and desalted. The integrity of the purified oligonucleotide was confirmed by LCMS. The LC/UV chromatogram and deconvoluted mass spectrum of the conjugate comprising (gGal) 3 conjugated at the 3′ end of an oligonucleotide matching the sense strand of inclisiran showed: retention time =5.927 min, target mass =8678.2 Da, observed mass =8676.8 Da, mass error=−1.4 Da (-0.016%), % estimated purity =64.62 % . Equimolar amounts of complementary sense and antisense strands were mixed and annealed by heating to 95° C. and slowly cooled to obtain the desired (gGal)3-siRNA (GalNAc conjugated siRNA 1). (gGal)3-siRNA conjugate: The conjugate comprising the duplex siRNA sequence of inclisiran and (gGal) 3 conjugated to the 3′ sense strand had retention time =8.975 min and % peak area =96.8%.

### Example 20—Synthesis and Characterisation of GalNAc Conjugated Oligonucleotide 2 ((ptGal)3-oligo) and GalNAc Conjugated siRNA 2 ((ptGal)3-siRNA)

Antisense strands were synthesized on a MerMade 48 synthesizer using commercially available 5′-O-(4,4′-dimethoxytrityl)-2′-deoxy-, 5′-O-(4,4′-dimethoxytrityl)-2′- deoxy-2′-fluoro-, and 5′-O-(4,4′-dimethoxytrityl)-2′-O-methyl-3′-O-(2-cyanoethyl-N,N-diisopropyl) phosphoramidite monomers of uridine, thymidine, 4-N-acetylcytidine, 6-N-benzoyladenosine, and 2-N-isobutyrylguanosine using standard solid-phase oligonucleotide synthesis and deprotection protocols. In sense strand synthesis, respective GalNAc CPG and GalNAc amidite monomers were used. The antisense strand and sense strand are those of inclisiran, corresponding to SEQ ID NO: 4 and 9, respectively.

Solid phase synthesis on the universal support was first introduced to couple with ptGal phosphoramidite in three reaction cycles successively and then followed with standard oligonucleotide synthesis. Phosphorothioate linkages were introduced by oxidation of phosphite utilizing 0.1 M DDTT in pyridine. After cleavage and deprotection with aqueous methylamine at 55° C. for 4 hours, the (ptGal)3-oligo (GalNAc conjugated oligonucleotide 2) was achieved in crude state and was further purified by by anion-exchange high-performance liquid chromatography (IEX-HPLC). The pure fractions were combined, concentrated, and desalted. The integrity of the purified oligonucleotide was confirmed by LCMS. The LC/UV chromatogram and deconvoluted mass spectrum of the conjugate comprising (ptGal)3 conjugated at the 3′ end of an oligonucleotide matching the sense strand of inclisiran showed: retention time=5.817 min, target mass=9089.1 Da, observed mass=9089.6 Da, mass error=0.5 Da (0.006%), % estimated purity=91.96%. Equimolar amounts of complementary sense and antisense strands were mixed and annealed by heating to 95° C. and slowly cooled to obtain the desired (ptGal)3-siRNA (GalNAc conjugated siRNA 2). The conjugate comprising the duplex siRNA sequence of inclisiran and (ptGal)₃ conjugated to the 3′ sense strand had retention time=8.936 min and % peak area=95.4%.

### Example 21—Synthesis and Characterisation of GalNAc Conjugated Oligonucleotide 3 ((tGal)3-oligo) and GalNAc Conjugated siRNA 3 ((tGal)3-SIRNA)

Antisense strands were synthesized on a MerMade 48 synthesizer using commercially available 5′-O-(4,4′-dimethoxytrityl)-2′-deoxy-, 5′-O-(4,4′-dimethoxytrityl)-2′- deoxy-2′-fluoro-, and 5′-O-(4,4′-dimethoxytrityl)-2′-O-methyl-3′-O-(2-cyanoethyl-N,N-diisopropyl) phosphoramidite monomers of uridine, thymidine, 4-N-acetylcytidine, 6-N-benzoyladenosine, and 2-N-isobutyrylguanosine using standard solid-phase oligonucleotide synthesis and deprotection protocols. In sense strand synthesis, respective GalNAc CPG and GalNAc amidite monomers were used. The antisense strand and sense strand are those of inclisiran, corresponding to SEQ ID NO: 4 and 10, respectively.

Solid phase synthesis on the universal support was first introduced to couple with tGal phosphoramidite in three reaction cycles successively and then followed with standard oligonucleotide synthesis. Phosphorothioate linkages were introduced by oxidation of phosphite utilizing 0.1 M DDTT in pyridine. After cleavage and deprotection with aqueous methylamine at 55° C. for 4 hours, the (tGal)3-oligo (GalNAc conjugated oligonucleotide 3) was achieved in crude state and was further purified by by anion-exchange high-performance liquid chromatography (IEX-HPLC). The pure fractions were combined, concentrated, and desalted. The integrity of the purified oligonucleotide was confirmed by LCMS.

The LC/UV chromatogram and deconvoluted mass spectrum of the conjugate comprising (tGal) 3 conjugated at the 3′ end of an oligonucleotide matching the sense strand of inclisiran showed: retention time =5.905 min, target mass =9100.6 Da, observed mass =9098.7 Da, mass error=−1.9 Da (-0.021%), % estimated purity =89.34%.

Equimolar amounts of complementary sense and antisense strands were mixed and annealed by heating to 95° C. and slowly cooled to obtain the desired (tGal)3-siRNA (GalNAc conjugated siRNA 3). The conjugate comprising the duplex siRNA sequence of inclisiran and (tGal) 3 conjugated to the 3′ sense strand had retention time =8.898 min and % peak area =96.1%.

### Example 22—In vitro Evaluation of GalNAc Monomer-siRNA Conjugates Inhibition Efficacy on PCSK9

(ptGal)3-SiRNA, (tGal)3-siRNA and (gGal)3-siRNA of Example 19 to 21 were evaluated for their ability to inhibit protein expression of PCSK9 in vitro, as follows.

Human hepatocyte carcinoma HuH7 cell line (Cellosaurus Accession: CVCL_0336) were cultured with DMEM high glucose supplemented with 10% FBS, 1% sodium pyruvate and 1% PenStrep in a humidified incubator at 37° C. and 5% CO₂.

The siRNAs tested were (ptGal)3-siRNA, (tGal)3-SiRNA, (gGal)3-SiRNA, clinical grade inclisiran (Leqvio®) and inclisiran synthesised in-house (triGaINAc-siRNA). The siRNA with working concentration of 10nM, 1nM, 0.5nM, 0.25nM, 0.125nM, 0.0625nM, 0.03125 nM, 0.015625 nM, 0.003125 nM, and 0.000625 nM were transfected into HuH-7 cells using RNAimax. PCSK9 protein in the supernatant was determined by ELISA after 3 days post-transfection. IC50 values were determined based on PCSK9 protein expression.

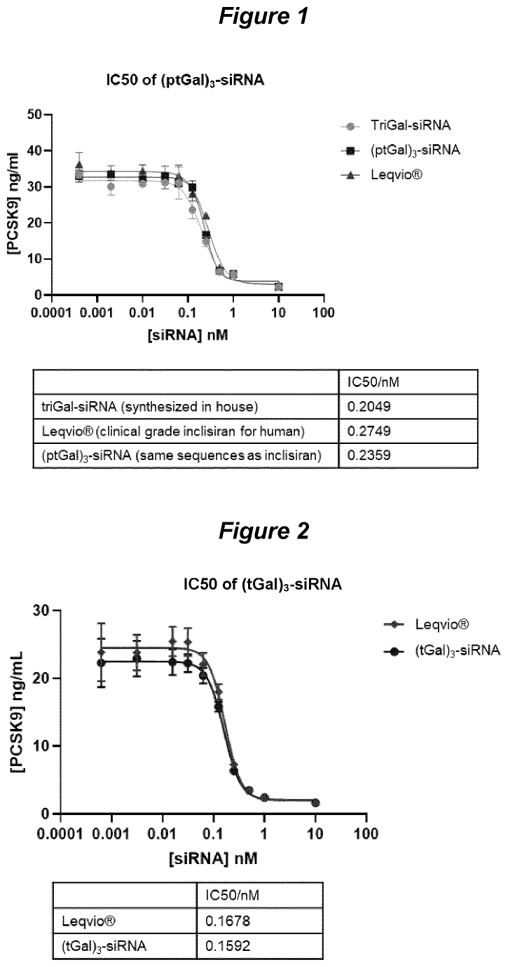

The results are shown in FIGS. 1, 2 and 3.

(ptGal)₃-siRNA shows similar IC50 value with clinical grade inclisiran (marketed as Leqvio®) and inclisiran synthesised in-house (labelled triGal-siRNA) (p>0.05). (FIG. 1).

(tGal)₃-siRNA shows similar IC50 value with clinical grade inclisiran (marketed as Leqvio®) (p>0.05) (FIG. 2).

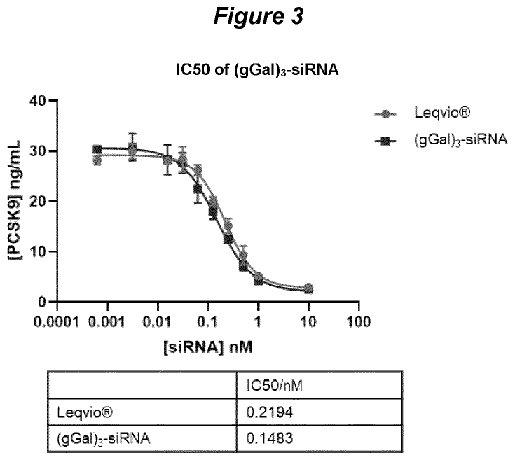

(gGal)₃-siRNA potently inhibits gene expression of PCSK9, with an IC50 of 0.1483 nM which is lower than the IC50 of inclisiran (maketed as Leqvio®) as determined in this experiment (FIG. 3).

Oligonucleotide conjugates ((ptGal)₃-siRNA, (tGal)₃-SiRNA, (gGal)₃-siRNA) targeting PCSK9 are able to potently knockdown PCSK9 in vitro and in some instances with greater potency than the triGaINAc-conjugated inclisiran.

### Example 23—In vivo Evaluation of GalNAc Monomer-siRNA Conjugates Inhibition Efficacy on PCSK9

(ptGal)₃-SiRNA, (tGal)₃-SiRNA and (gGal)₃-siRNA were tested for their ability to reduce PCSK9 protein production in hPCSK9-Knock-in mice.

The siRNAs were administrated through subcutaneous injection at single dose of 2 mg/kg. The hPCSK9 protein serum levels were determined by ELISA. The siRNA effects on PCSK9 protein reduction were determined up to 49 days post-injection for (ptGal)₃-SIRNA, up to 48 days post-injection for tGal-siRNA, and up to 32 days post-injection for (gGal)₃-SiRNA. Each siRNA was compared with Leqvio®, and in the case of (gGal)₃-SiRNA, also with unconjugated siRNA. Unconjugated siRNA having the oligonucleotide sequence of inclisiran but not bearing any GalNAc moiety was used as a negative control.

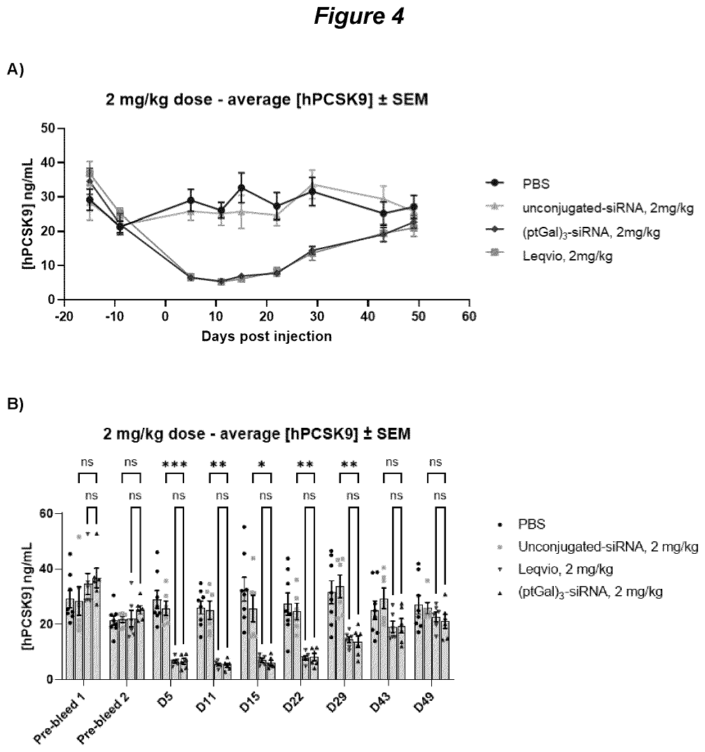

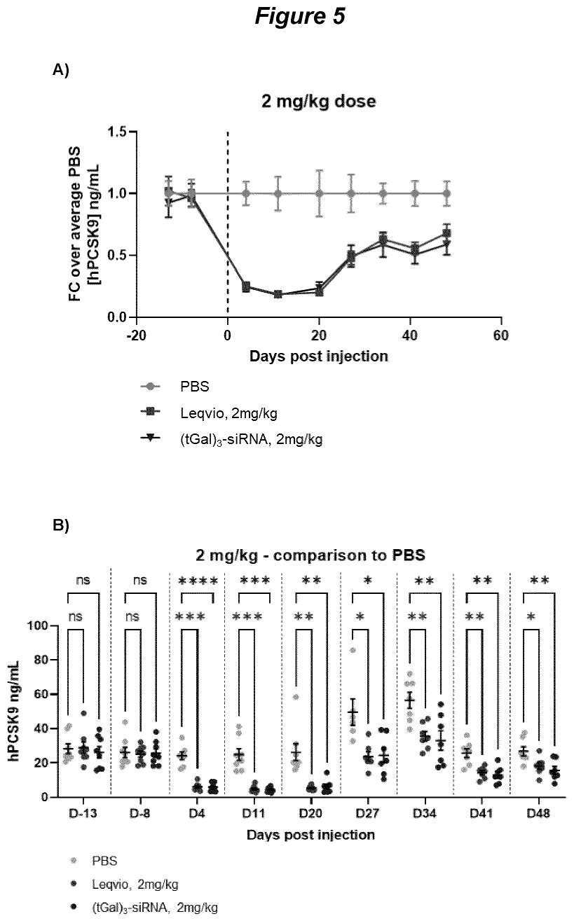

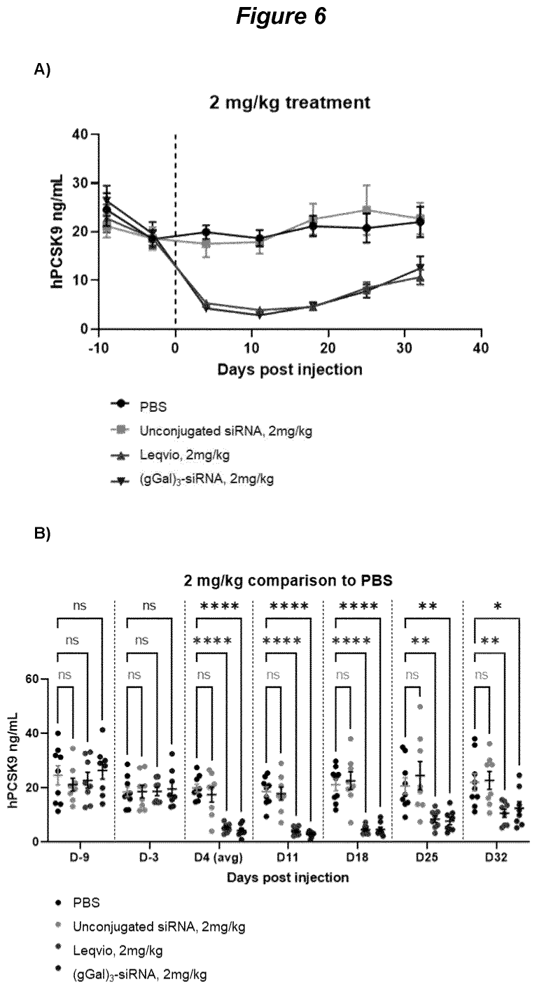

FIGS. 4A-4B, 5A-5B and 6A-6B show that the in vivo efficacy and duration of siRNAs when administered at 2 mg/kg. The knockdown efficacy of hPCSK9 protein production from the GalNAc conjugates according to the invention was similar to the efficacy of Leqvio® as no statistically significant difference in the efficacy and duration was observed. As expected, the unconjugated siRNA was not able to regulate hPSCK9 production, as it is not able to reach liver hepatocytes.

Oligonucleotide conjugates ((ptGal)₃-SiRNA, (tGal)₃-siRNA, (gGal)₃-SiRNA) targeting PCSK9 are able to be delivered into hepatocytes and therefore potently decrease hPCSK9 protein production in vivo.

The inventors also observed desirable lack of liver toxicity in hPCSK9-Knock-in mice treated with the oligonucleotide conjugates ((ptGal)₃-SiRNA, (tGal)₃-SIRNA, (gGal)₃-SiRNA).

Sequences

| SEQ ID NO: | DESCRIPTION | SEQUENCE |
|---|---|---|
| 1 | Inclisiran sense strand | CUAGACCUGUTUUGCUUUUGU |
| | (passenger strand) | |
| | without triGalNAc | |
| | (unconjugated, without | |
| | modifications) | |
| 2 | Inclisiran antisense | ACAAAAGCAAAACAGGUCUAGAA |
| | strand (guide strand, | |
| | without modifications) | |
| 3 | Inclisiran sense strand | (mC)ps(mU)ps(mA)(mG)(mA)(mC)(fC)(mU)(fG)(mU)(dT)(mU)(mU) |
| | (passenger strand) | (mG)(mC)(mU)(mU)(mU)(mU)ps(mG)ps(mU) |
| | without triGalNAc | |
| | (unconjugated) | |
| | showing modifications | |
| 4 | Inclisiran antisense | (mA)ps(fC)ps(mA)(fA)(fA)(fA)(mG)(fC)(mA)(fA)(mA)(fA)(mC) |
| | strand (guide strand) | (fA)(mG)(fG)(mU)(fC)(mU)(mA)(mG)ps(mA)ps(mA) |
| | showing modifications | |
| 5 | Inclisiran target | UUCUAGACCUGUUUUGCUUUUGU |
| 6 | Human PCSK9 isoform | MGTVSSRRSWWPLPLLLLLLLLLGPAGARAQEDEDGDYEELVLA |
| | 1 (UniProtKB: | LRSEEDGLAEAPEHGTTATFHRCAKDPWRLPGTYVVVLKEETHL |
| | Q8NBP7-1, v3) | SQSERTARRLQAQAARRGYLTKILHVFHGLLPGFLVKMSGDLLEL |
| | | ALKLPHVDYIEEDSSVFAQSIPWNLERITPPRYRADEYQPPDGGS |
| | | LVEVYLLDTSIQSDHREIEGRVMVTDFENVPEEDGTRFHRQASKC |
| | | DSHGTHLAGVVSGRDAGVAKGASMRSLRVLNCQGKGTVSGTLI |
| | | GLEFIRKSQLVQPVGPLVVLLPLAGGYSRVLNAACQRLARAGVVL |
| | | VTAAGNFRDDACLYSPASAPEVITVGATNAQDQPVTLGTLGTNF |
| | | GRCVDLFAPGEDIIGASSDCSTCFVSQSGTSQAAAHVAGIAAMML |
| | | SAEPELTLAELRQRLIHFSAKDVINEAWFPEDQRVLTPNLVAALPP |
| | | STHGAGWQLFCRTVWSAHSGPTRMATAVARCAPDEELLSCSSF |
| | | SRSGKRRGERMEAQGGKLVCRAHNAFGGEGVYAIARCCLLPQA |
| | | NCSVHTAPPAEASMGTRVHCHQQGHVLTGCSSHWEVEDLGTH |
| | | KPPVLRPRGQPNQCVGHREASIHASCCHAPGLECKVKEHGIPAP |
| | | QEQVTVACEEGWTLTGCSALPGTSHVLGAYAVDNTCVVRSRDV |
| | | STTGSTSEGAVTAVAICCRSRHLAQASQELQ |
| 7 | Inclisiran sense strand | (mC)ps(mU)ps(mA)(mG)(mA)(mC)(fC)(mU)(fG)(mU)(dT)(mU)(mU) |
| | (Passenger strand) | (mG)(mC)(mU)(mU)(mU)(mU)(mG)(mU)(triGal) |
| | with triGalNAc | |
| 8 | (gGal)₃-oligo | (mC)ps(mU)ps(mA)(mG)(mA)(mC)(fC)(mU)(fG)(mU)(dT)(mU)(mU) |
| | | (mG)(mC)(mU)(mU)(mU)(mU)(mG)(mU)(gGal)ps(gGal)ps(gGal) |
| 9 | (ptGal)₃-oligo | (mC)ps(mU)ps(mA)(mG)(mA)(mC)(fC)(mU)(fG)(mU)(dT)(mU)(mU) |
| | | (mG)(mC)(mU)(mU)(mU)(mU)(mG)(mU)(ptGal)ps(ptGal)ps(ptGal) |
| 10 | (tGal)₃-oligo | (mC)ps(mU)ps(mA)(mG)(mA)(mC)(fC)(mU)(fG)(mU)(dT)(mU)(mU) |
| | | (mG)(mC)(mU)(mU)(mU)(mU)(mG)(mU)(tGal)ps(tGal)ps(tGal) |

In the sequences of the table above, G = guanosine-3′-phosphate, C = cytidine-3′-phosphate, U = uridine-3′-phosphate, T = thymidine-3′-phosphate, A = adenosine-3′-phosphate, dA = deoxyadenosine-3′-phosphate, dT = deoxythymidine-3′-phosphate, dU = deoxyuridine-3′-phosphate, mU = 2′-O-methyluridine-3′-phosphate, mA = 2′-O-methyladenosine-3′-phosphate, mG = 2′-O-methylguanosine-3′-phosphate, mC = 2′-O-methylcytidine-3′-phosphate, (mU)ps = 2′-O-methyluridine-3′-phosphorothioate, (mA)ps = 2′-O-methyladenosine-3′-phosphorothioate, (mG)ps = 2′-O-methylguanosine-3′-phosphorothioate, (mC)ps = 2′-O-methylcytidine-3′-phosphorothioate, fU = 2′-fluorouridine-3′-phosphate, fA = 2′-fluoroadenosine-3′-phosphate, fG = 2′-fluoroguanosine-3′-phosphate, fC = 2′-fluorocytidine-3′-phosphate, (fC)ps = 2′-fluorocytidine-3′-phosphorothioate, (fG)ps = 2′-fluoroguanosine-3′-phosphorothioate, (fA)ps = 2′-fluoroadenosine-3′-phosphorothioate, (fU)ps = 2′-fluorouridine-3′-phosphorothioate, ps = 3′-phosphorothioate, (TriGal) = Triantennary N-acetylgalactosamine, (gGal) = a monomer of Formula 2, for example a monomer of formula (C), (ptGal) = a monomer of Formula 3a, for example a monomer of formula (A), and (tGal) = a monomer of Formula 3, for example a monomer of formula (B).

### REFERENCES

A number of publications are cited above in order to more fully describe and disclose the invention and the state of the art to which the invention pertains. Full citations for these references are provided below. The entirety of each of these references is incorporated herein.
U.S. Pat. No. 8,106,022 B2
U.S. Pat. No. 10,781,175 B2
U.S. Pat. No. 10,131,807 B2
U.S. Pat. No. 10,781,175 B2
WO 2021/032777 A1
Hofmeister, A. et al. J. Med. Chem. 2021, 64, 6838-6855.

1. A compound of Formula 3: 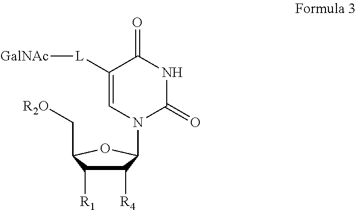
or a pharmaceutically acceptable salt thereof;
wherein
R₁ is O-PN(C1.4alkyl)₂OCH₂CH₂CN, OH, a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage;
R₂ is H or a protecting group;
R₄ is H, OH, OC₁₋₄alkyl or halogen;
L is —(W—Y)ₖ—W—X—;
k is 0 to 5;
each W is independently L1 or L2;
each L1 is (CH₂)ₙ, where n is independently 1 to 25;
each L2 is CH₂CH₂(HetCH₂CH₂)ₘ, where m is independently 1 to 24, and Het is independently a heteroatom;
X is a bond, Het, —CH₂—, —CO—, *O—CH₂—CO, *—(Het)CH₂C≡C—, or *—CH₂C≡C—, where * if present denotes the point of attachment to W; and
each Y is independently CONZ, O—CH₂—CONZ, NZCO, SO₂NZ, O—CH₂SO₂NZ or NZSO₂, where Z is H, C₁₋₄alkyl or a protecting group; and wherein
GalNAc may be protected may be deprotected.

2. A compound of Formula 2: 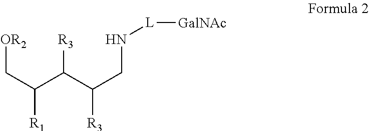
or a pharmaceutically acceptable salt thereof;
wherein
R₁ is O-PN(C1.4alkyl)₂OCH₂CH₂CN, OH, a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage;
R₂ is H or a protecting group;
each R₃ is independently C₁₋₄alkyl, OC₁₋₄alkyl, C₁₋₄haloalkyl, OC₁₋₄haloalkyl or H;
L is —(W—Y)ₖ—W—X—;
k is 0 to 5;
each W is independently L1 or L2;
each Ll is (CH₂)ₙ, where n is independently 1 to 25;
each L2 is CH₂CH₂(HetCH₂CH₂)ₘ, where independently m is 1 to 24, and Het is independently a heteroatom;
X is a bond, Het, —CH₂—, —CO—, *O—CH₂—CO, *—(Het)CH₂C≡C—, or *—CH₂C≡C—, where * if present denotes the point of attachment to W; and
each Y is independently CONZ, O—CH₂—CONZ, NZCO, SO₂NZ, O—CH₂SO₂NZ or NZSO₂, where Z is H, C₁₋₄alkyl or a protecting group; and wherein
GalNAc may be protected may be deprotected.

3. (canceled)

4. The compound according to claim 1, wherein the compound is a monomer for oligonucleotide synthesis and R₁ is

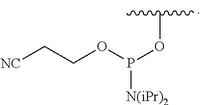

5. The compound according to claim 1, wherein:
L is -L1-X—, -L1-Y-L1-X—, -L1-Y-L2-X—, -L2-X—, -L2-Y-L1-X—, or -L2-Y-L2-X-;
each L1 is (CH₂)ₙ, where n is independently 2 to 10;
each L2 is CH₂CH₂(OCH₂CH₂)ₘ, where m is independently 1 to 5;
X is a bond, —CH₂—, —CO—, —O—, *—(Het)CH₂C≡C—, or *—CH₂C≡C—, where * if present denotes the point of attachment to W; and
Y is CONZ, where Z is H, C₁₋₄alkyl or a protecting group.

6. The compound according to claim 1, wherein in each L1, if present, n is independently 4 to 9.

7. The compound according to claim 1, wherein in each L2, if present, Het is —O—.

8. The compound according to claim 7, wherein m is 1, 2, 3, or 4; optionally wherein m is 1, 2 or 3.

9. The compound according to claim 2, wherein X is —CO—.

10. The compound according to claim 1, wherein X is —O—.

11. The compound according to claim 1, wherein L (or L′, if present) is —(CH₂)₂O—, —(CH₂)₄—CO—, —(CH₂)₉—CO—, —(CH₂)₄—CONH—CH₂CH₂OCH₂CH₂—, —(CH₂)₅—CONH—(CH₂)₄—CO—, —(CH₂)₄—CONH—CH₂CH₂(OCH₂CH₂)—O—, or —(CH₂)₄—CONH—CH₂CH₂(OCH₂CH₂)—O—(CH₂)C≡C—.

12. The compound according to claim 1, wherein the compound is selected from: 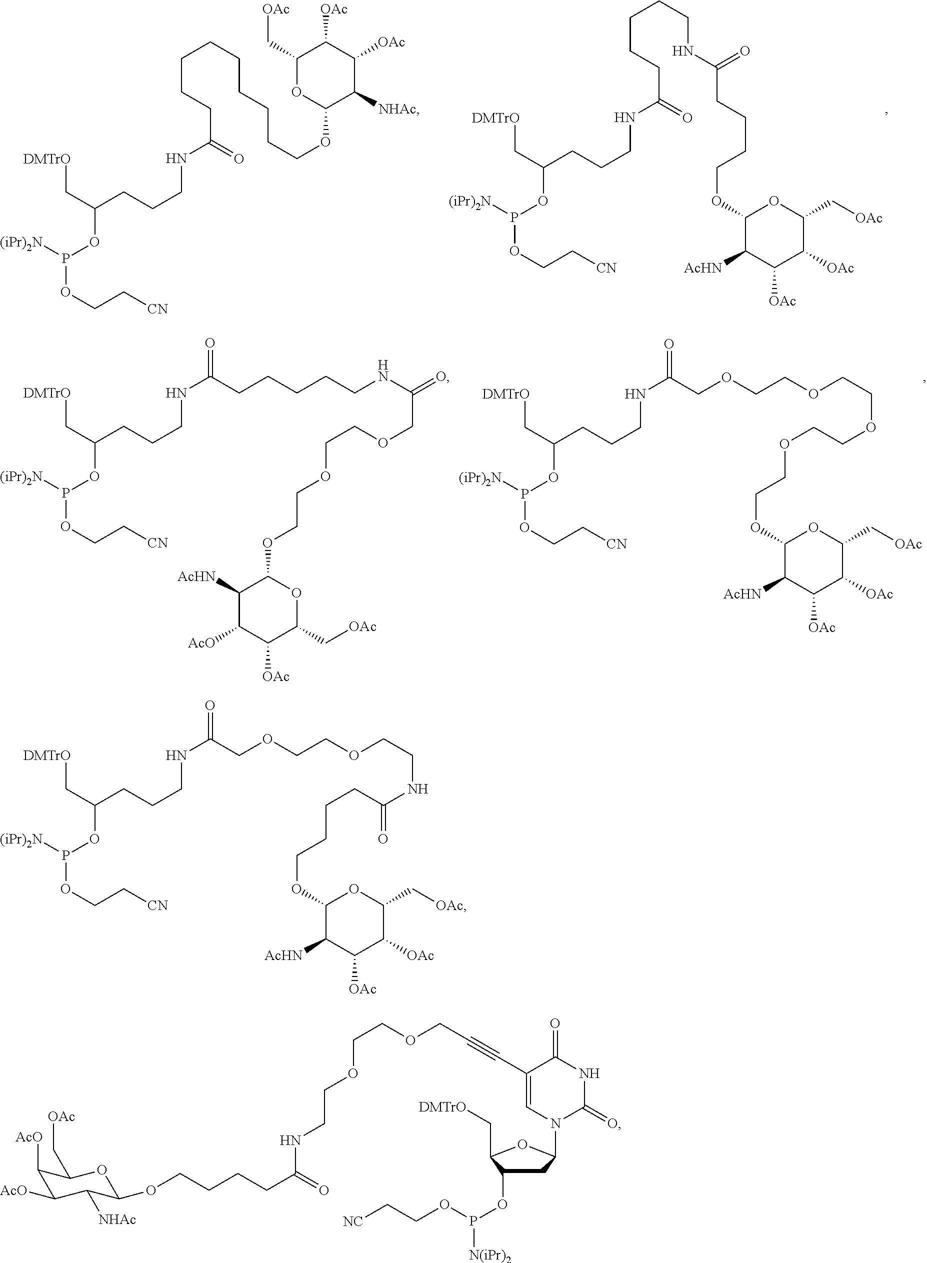 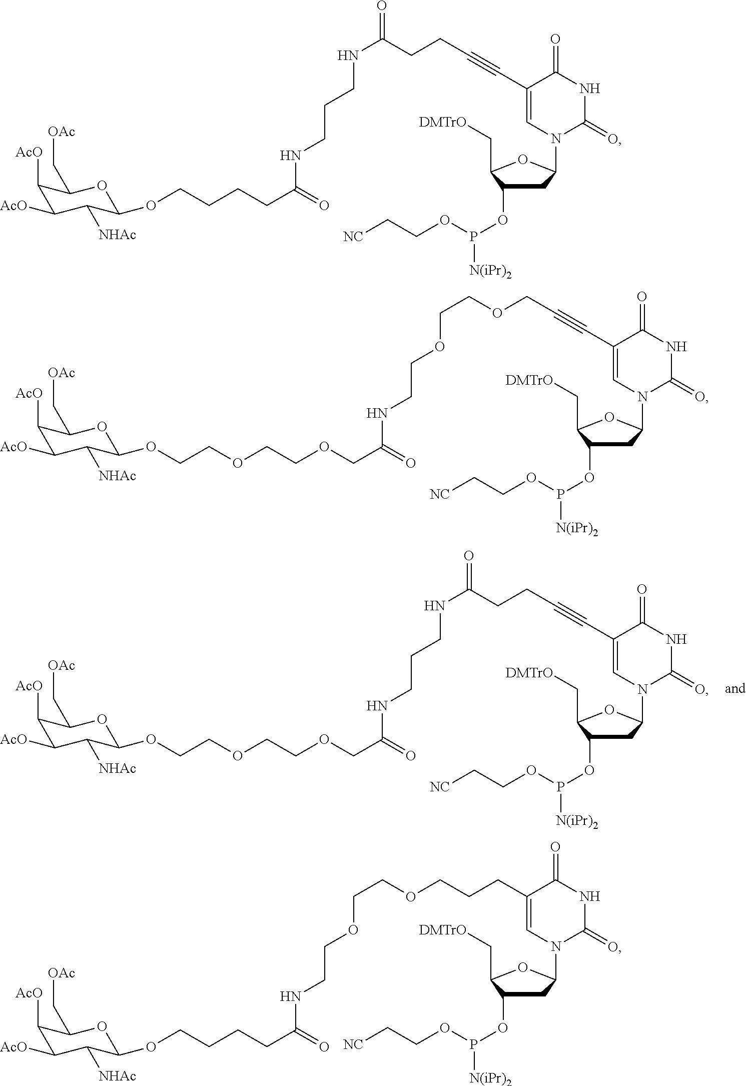
and, where the compound contains an alkyne bond, the corresponding compound in which that bond is fully hydrogenated;
and pharmaceutically acceptable salts thereof.

13. The compound according to claim 1, wherein the compound is selected from: 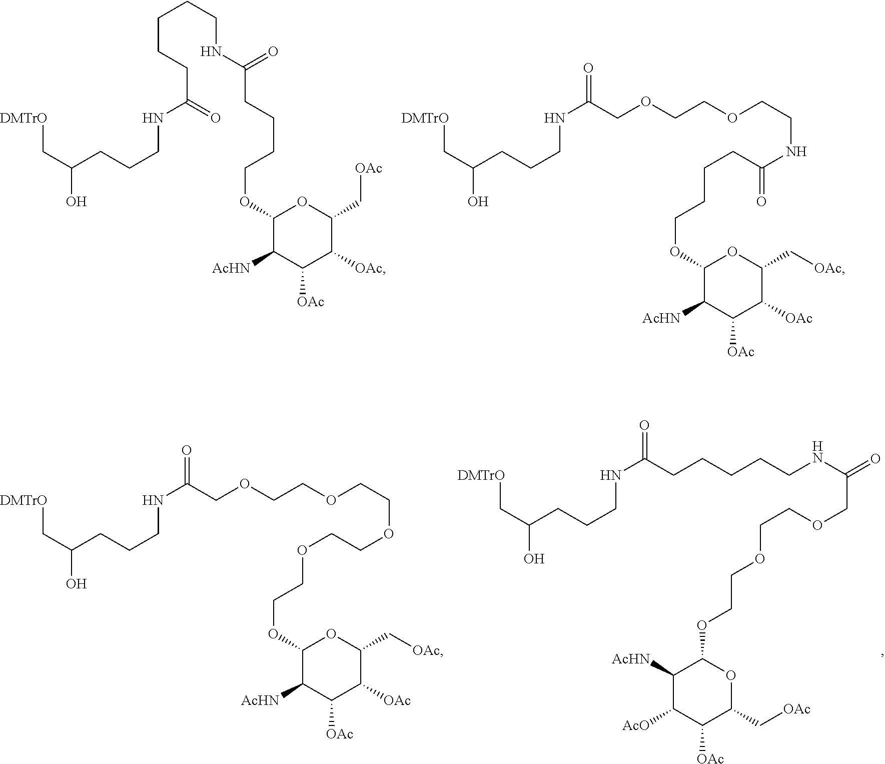 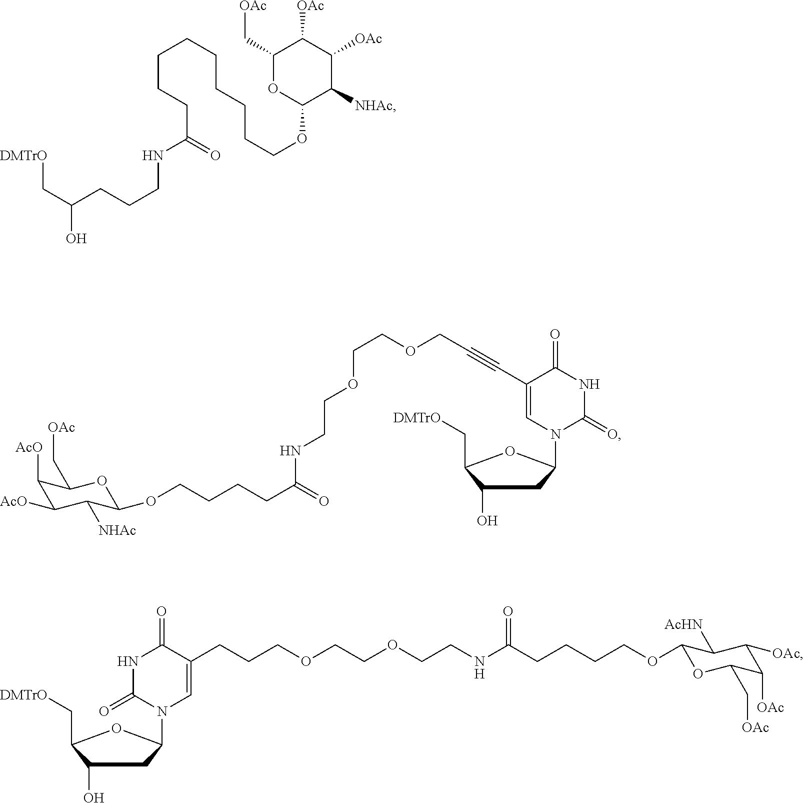 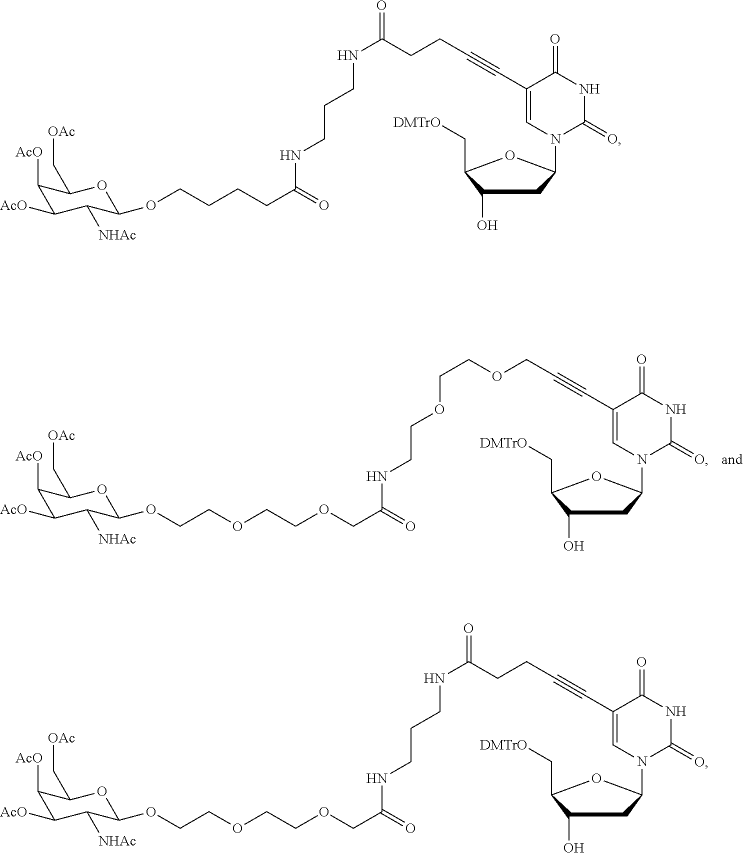
and, where the compound contains an alkyne bond, the corresponding compound in which that bond is fully hydrogenated;
and salts thereof.

14. The compound according to claim 1, wherein R₁ is a polystyrene bead or long chain alkylamine controlled-pore glass (LCAA-CPG) which is connected through a succinic acid linkage, and optionally wherein the compound is selected from: 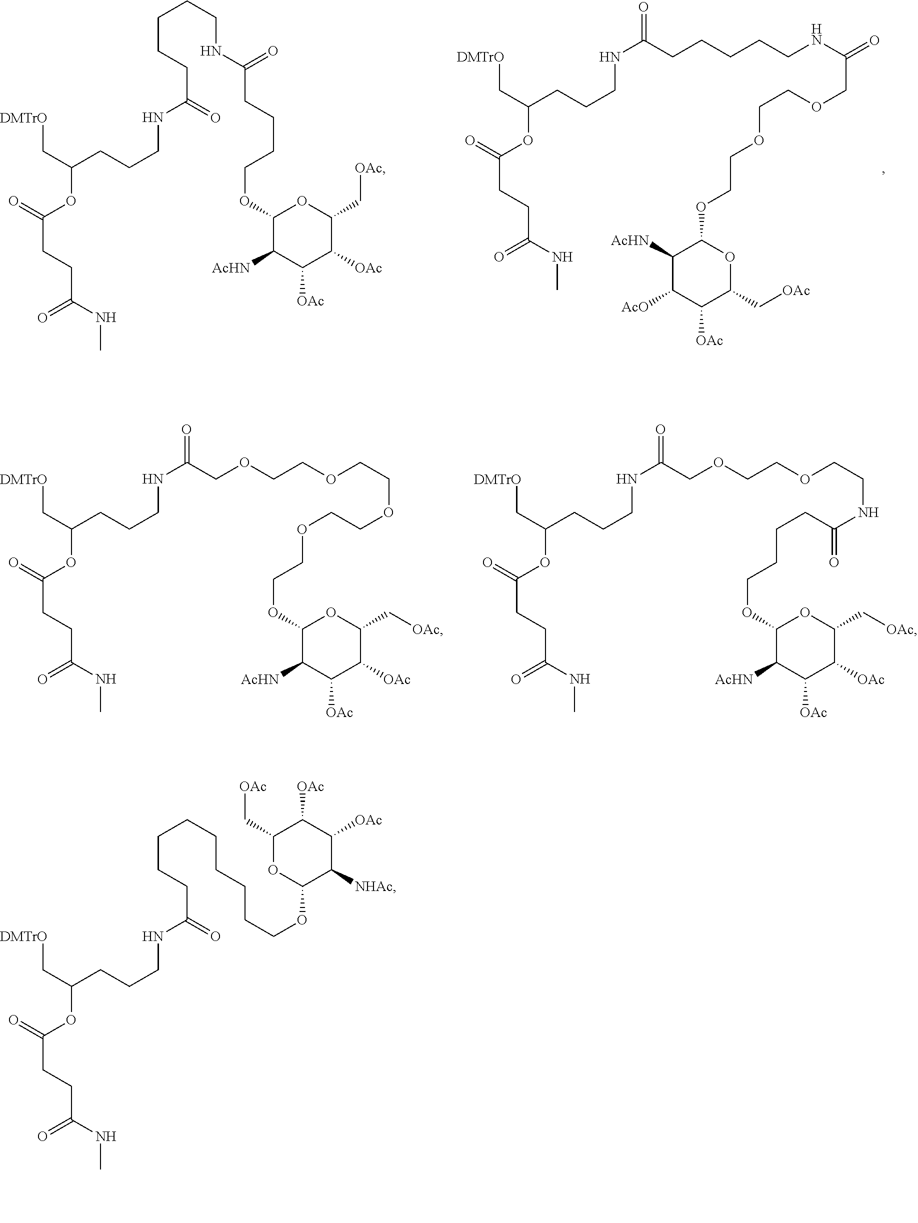 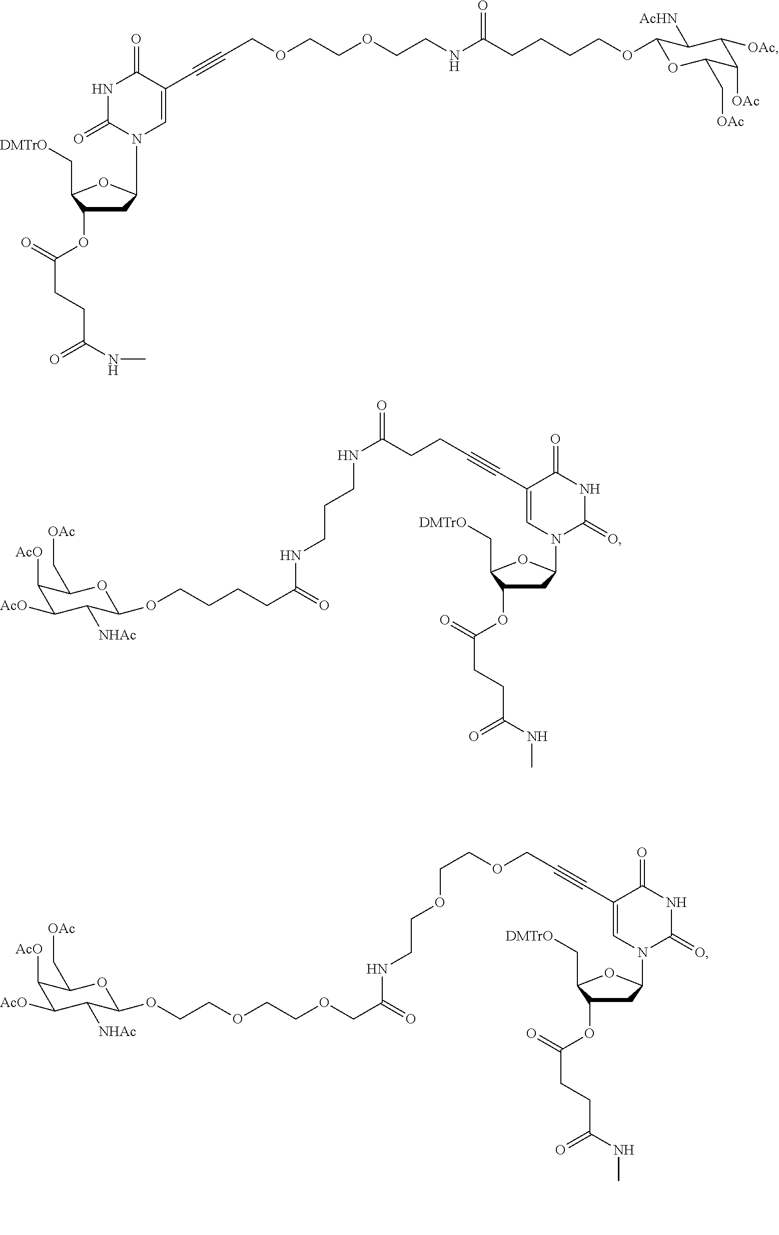 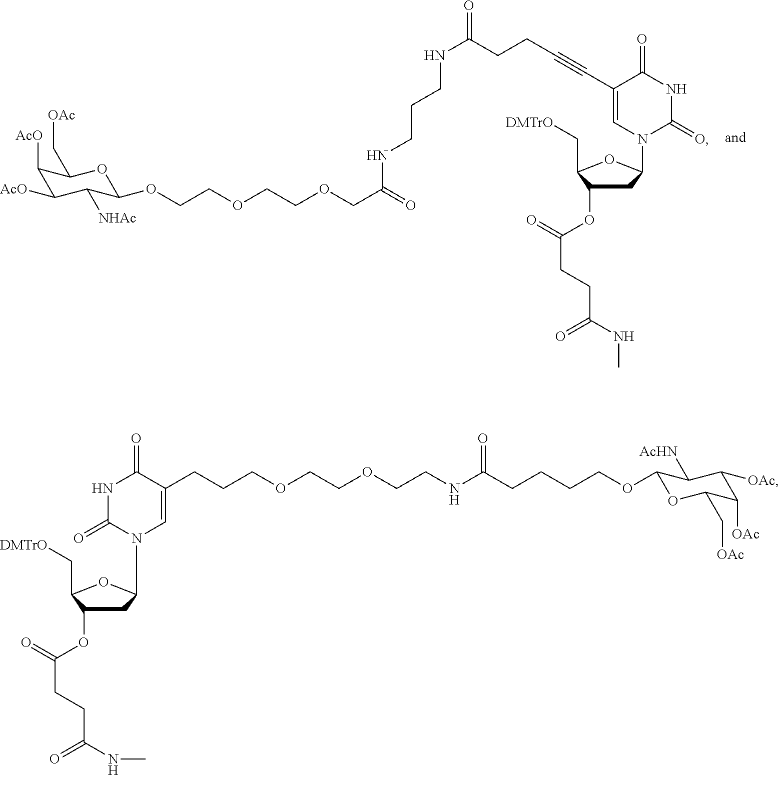
wherein the circle represents the LCAA-CPG or polystyrene bead, and salts thereof;
and, where the compound contains an alkyne bond, the corresponding compound in which that bond is fully hydrogenated.

15. (canceled)

16. An oligonucleotide comprising at least one monomer residue of formula: 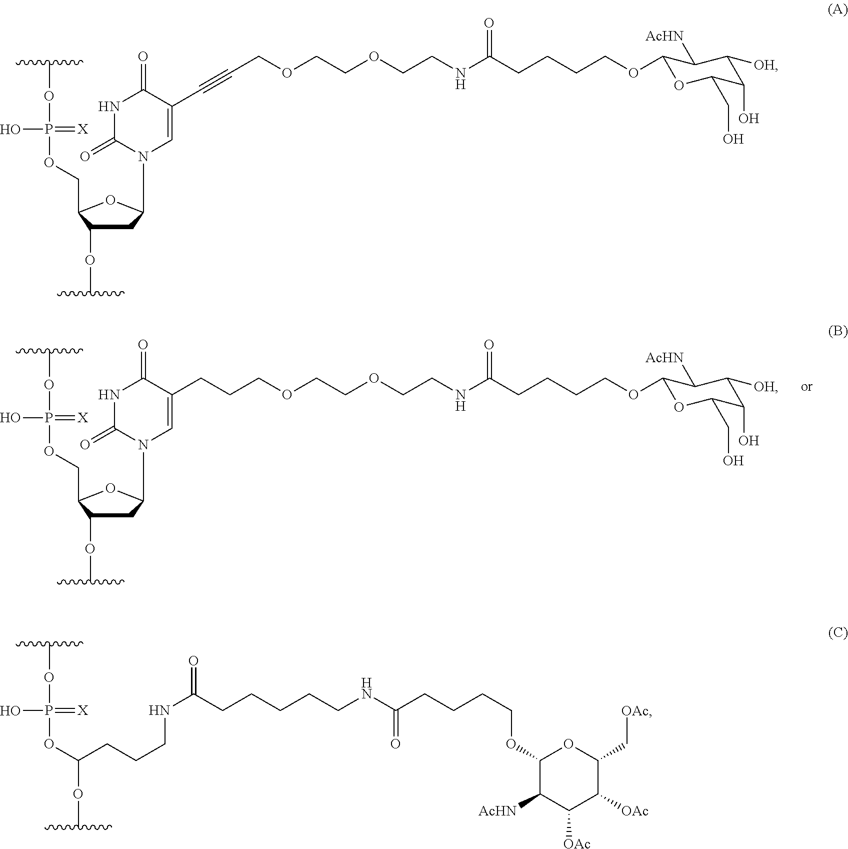
wherein X is S or O.

17. The oligonucleotide of claim 16 comprising, optionally at the 3′-end, three successive copies of a monomer residue of formula: 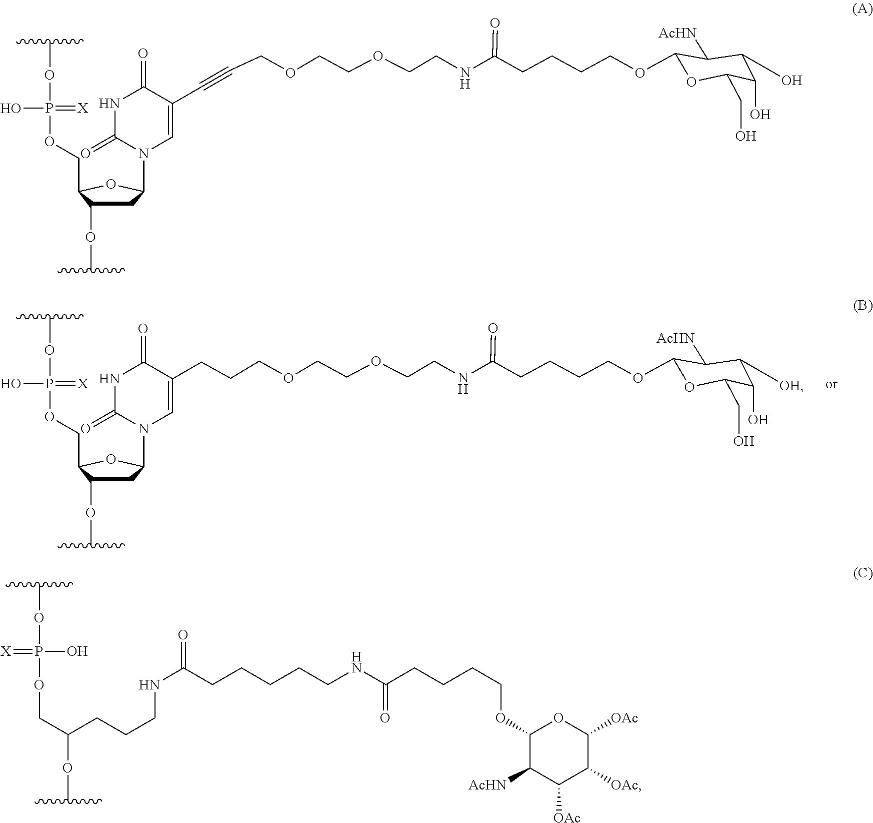
wherein X is S or O.

18. The oligonucleotide of claim 16, wherein X is S.

19. An oligonucleotide of claim 16 selected from: 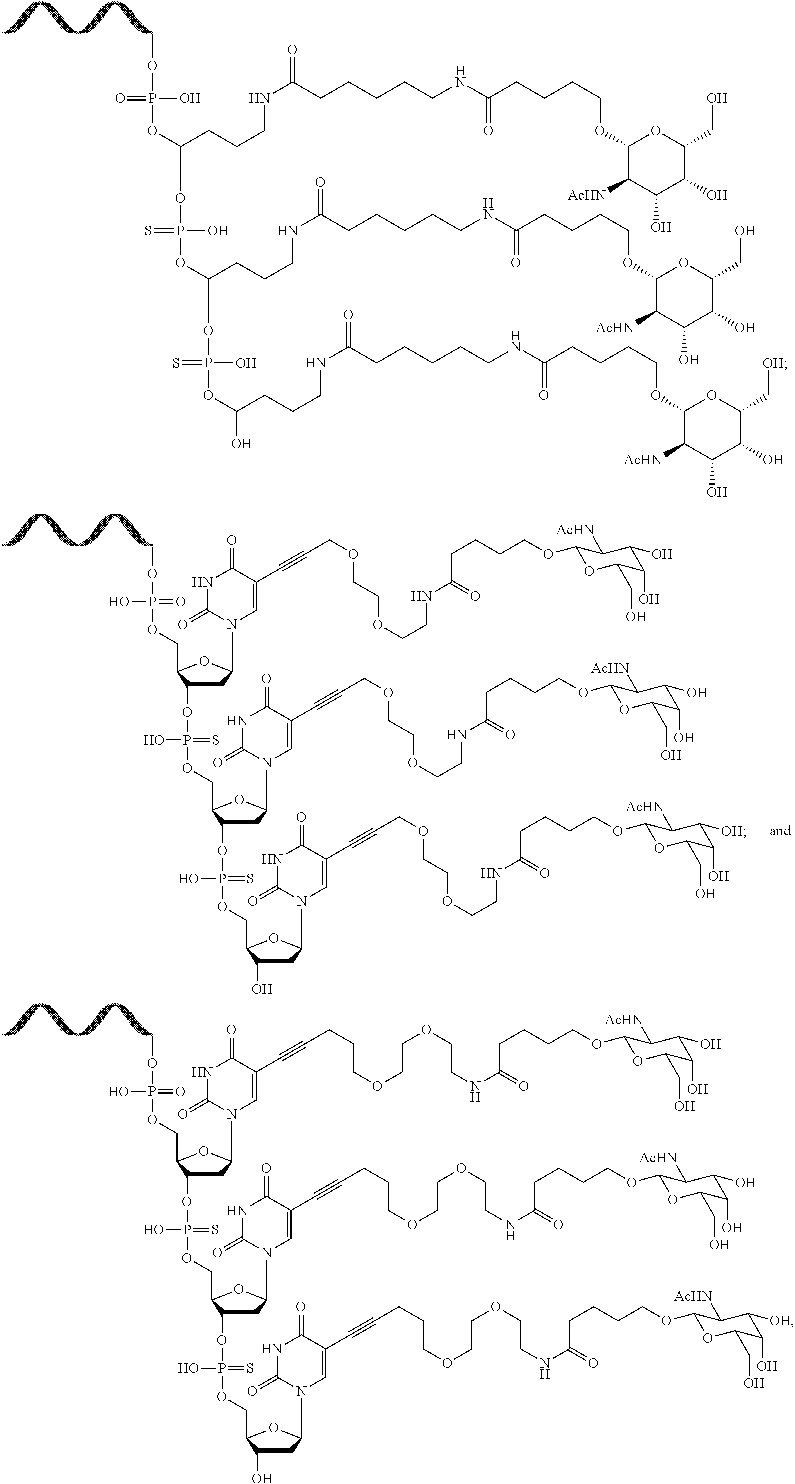
wherein the waved line indicates an oligonucleotide chain.

20. (canceled)

21. 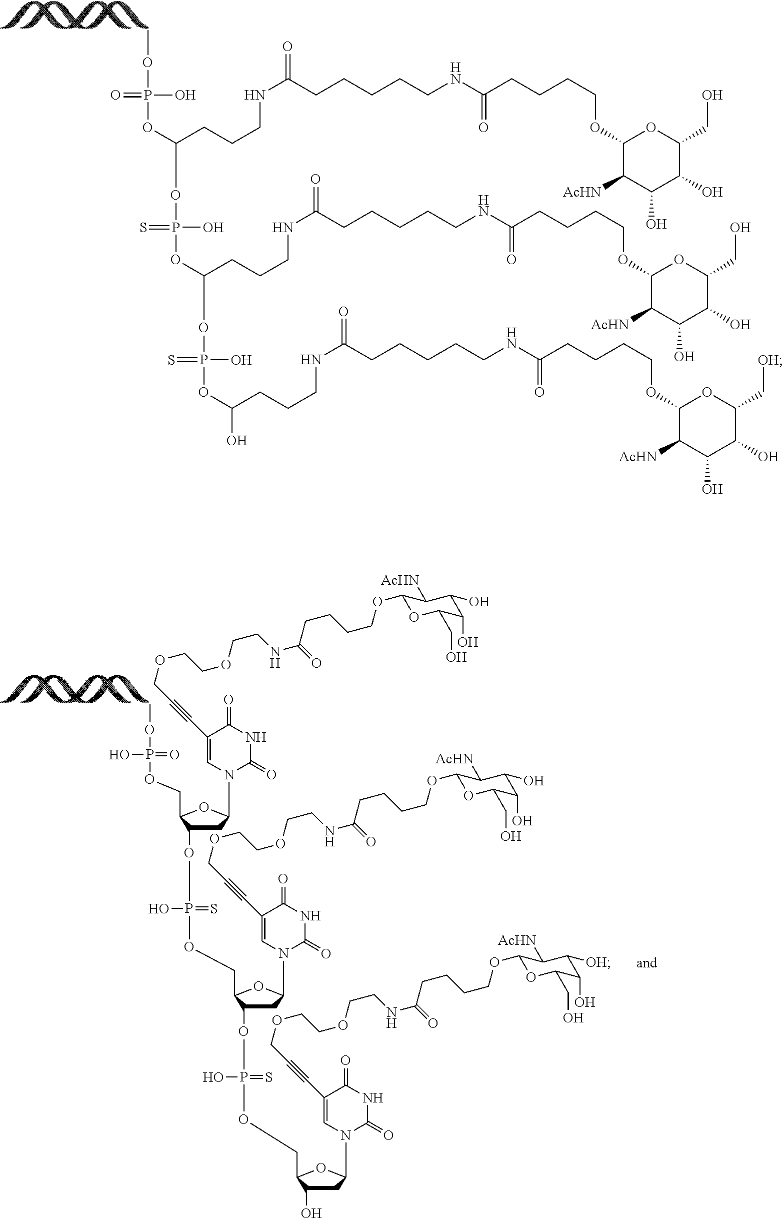 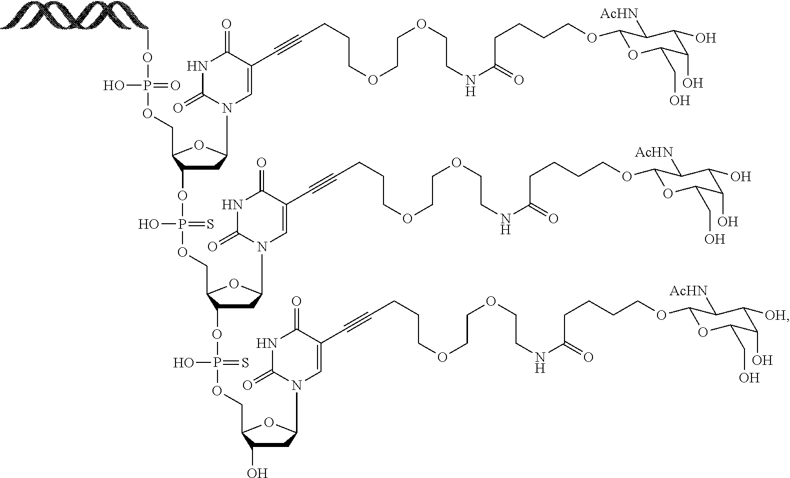
wherein the waved lines indicate RNA strands.
